(19) United States
(12) Patent Application Publication
(10) Pub. No.: US 2013/0324848 A1
(43) Pub. Date: Dec. 5, 2013

(54) BIOMETRIC INFORMATION MEASURING DEVICE AND BIOMETRIC INFORMATION MEASURING SYSTEM

(71) Applicants: Shigehiro Kuroki, Shimotsuke-shi (JP); Shiro Yamashita, Ichikawa-shi (JP)

(72) Inventors: Shigehiro Kuroki, Shimotsuke-shi (JP); Shiro Yamashita, Ichikawa-shi (JP)

(21) Appl. No.: 13/512,291

(22) PCT Filed: Nov. 22, 2010
(86) PCT No.: PCT/JP2010/071206

§ 371 Date: Jun. 7, 2013

(30) Foreign Application Priority Data
Nov. 25, 2009 (JP) 2009-267156

(51) Int. Cl. A61B 5/0245; A61B 5/044; A61B 7/00; A61B 5/04; A61B 5/024; A61B 8/04; A61B 8/02; A61B 8/00; A61B 8/08; A61B 5/021; A61B 5/00

(52) U.S. Cl. CPC A61B 5/0245; A61B 5/021; A61B 5/044; A61B 7/00; A61B 5/04012; A61B 5/7475; A61B 8/04; A61B 8/02; A61B 8/461; A61B 8/5223; A61B 5/02416
USPC 600/438; 600/513; 600/485; 600/453; 600/479

(57) ABSTRACT

Though mechanocardiogram is considered to have a high medical value, mechanocardiogram recording device is large in size and very expensive, obtained data lacks reliability, a measurement algorithm has not been settled, use of an apexcardiogram for diagnosis is not useful for diagnosis of the circulatory system, and there has been no measuring equipment capable of measuring pexcardiogram which contribute to determination of heal condition of a living body to be measured. For measurement of heartbeat of a living body, measuring device which allows simple measurement of apexcardiogram at bedside has been developed using a pressure sensor and/or wave sensor capable of measuring change in pressure at multiple portions adjacent to one another, and simple electronic circuit. Data processing algorithm for obtaining health information of a living body to be measured with high reliability from measured apexcardiogram and a biometric information measuring device having a new data processing algorithm on board have been developed to solve the problem.

### TECHNICAL FIELD

The present invention relates to a biometric information measuring device, a biometric information measurement system, and a biometric information measuring method for grasping the motion of the heart of a living body, thereby assessing the health condition of the living body.

Note that in the following description, biometric information in principle relates to information obtained from the heart of a living body, such as the motion of the heart of the living body, motion of the heart influenced by blood flow, blood pressure, body temperature and the like, and the size of the heart. And health information relates to information of the health condition of the living body, which can be obtained or assessed based on the biometric information.

### BACKGROUND ART

In recent years, remarkable development of medical equipment due to the progress not only in medicine itself but also engineering and various types of measuring instruments in many fields have been developed. For example, at sites such as a hospital where medical professionals are deeply involved, many kinds of data, such as blood pressure of upper limbs, crural blood pressure, carotid arterial pulse, femoral arterial pulse, peripheral arterial pulse of the limbs, are taken and stored in a memory device and available for diagnosis.

On the other hand, while the emergence of practical measuring devices has been desired, there are some fields in which development such as for the circulatory system has not necessarily progressed.

In the case of the circulatory system, carotid arterial pulsation in the thorax, the abdomen, and the cervix (cervical vein) can already be measured and stored as a carotid arterial pulse using a measuring device, and available for diagnosis. However, there is no available effective measuring device for diagnostic apexcardiograms other than for the carotid arterial pulse.

Nowadays, the number of diseases of the circulatory system is steadily increasing and there has been a global outcry for overcoming them.

The heart is a very important organ repeatedly contracting approximately thirty million times per year and continuing to move for decades, and repeats complicated movement inside the breastbone and ribs while being affected by other organs. It is known that such movement differs from person to person and also differs according to each health condition thereof.

There are many examples of diseases of the circulatory system, for which improvement in medical technology and preventive medical technology is desired, including hypertonia, hypertensive heart disease, acute myocardial infarction, old myocardial infarction, hypertrophic cardiomyopathy, dilated cardiomyopathy, secondary cardiomyopathy, aortic stenosis, aortic regurgitation, mitral stenosis, mitral regurgitation, arteriosclerosis obliterans, chronic heart failure, arrhythmia, etc.

If such a measuring device capable of grasping the motion of the heart objectively is available, the health condition of a measured living body can be recognized with a fair degree of precision and it can thus be used for diagnosis and treatment. For example, the systole motion of the heart is greatly influenced by his/her health condition. There are cases where specialists having advanced knowledge of the circulatory system may be able to diagnose pathological conditions by applying a hand to a patient's chest and palpating.

Inspection, palpation, and auscultation are important for medical treatment. Inspection, palpation, and auscultation for diagnosis of the heart provide medical practitioners with many important kinds of information. Notably, palpation will give important information useful for correct diagnosis. Palpation, however, is difficult to provide an objective expression and is thus a big issue that should be improved even in diagnosis and clinical medicine education.

The apexcardiogram is one of the means for objectively expressing information of the motion of the heart obtained through palpation. If a measuring device for conducting apexcardiography for measured living bodies, including not only healthy persons but also patients having a heart disease and having limited activity, at the bedside in an ordinary room instead of a soundproof room without making them be nervous is available, this should be great news for health care and medical treatment of measured living bodies.

However, a practical measuring device capable of precise measurement for apexcardiograms of human beings as measured living bodies has regrettably not been developed yet. Apexcardiography was attempted using the mechanocardiogram recording device disclosed in Non-patent Document 1 titled “Practice of examination of electrocardiogram and mechanocardiogram”. However, as described later, it was determined as unusable at medical sites and for individual health care.

FIG. 44 shows the conventional mechanocardiogram recording device disclosed in Non-patent Document 1, which is large having width of approximately 60 cm, height of approximately 180 cm, and depth of approximately 80 cm and equipped with a cart. However, it is still too heavy for a single person to move. In the figure, a window on the right hand side of the mechanocardiogram recording device main body is used to allow for looking into a soundproof room in which there is a living body to be measured. FIG. 45 shows exemplified photographs of various transducers described on page 212 of Non-patent Document 1.

The conventional mechanocardiogram recording device is expensive, costing approximately 23 million yen. In addition to this it requires, as described in Non-patent Document 1, a special environment or a soundproof room to conduct apexcardiogram, and thus requires an examiner to have advanced measuring techniques as well as advanced diagnostic capability as a medical specialist. Apexcardiogram requires a living body to be measured to enter a soundproof room, as well as at least one examiner to be in the soundproof room and at least one person to be on the outside. In many cases, at least two persons: a medical practitioner and a medical engineer are necessary for apexcardiogram.

The device in FIG. 44 has problems for apexcardiogram: it is impossible to measure unless a living body to be measured enters a soundproof room or a similar measuring room with little noise, and thus measurement should be performed in a restricted soundproof room like a sealed room, thereby making the measured living body be unnecessarily nervous due to the unusual environment in which apexcardiogram is conducted; the living body to be measured is limited to persons who can endure such a measurement in that environment; and it is impossible to conduct apexcardiogram for patients who are considered to unquestionably require diagnosis based on the apexcardiogram.

The inventor of the present invention considers that an inexpensive, small-sized apexcardiogram recording device capable of conducting apexcardiogram at the ordinary bedside with high reliability and allowing objective evaluation would be great news for medical care of the circulatory system. However, it is not yet known what such a device should be like, and apexcardiogram is considered very difficult even among specialists. Moreover, they consider that information acquired from measured data is insufficient, and applicability value is low, and therefore motivation of the medical equipment manufacturers is not very high.

This may be understood from the fact that remuneration for medical examinations conducted at medical care institutions is set extremely low. Namely, when an apexcardiogram is recorded using the conventional mechanocardiogram recording device, corresponding medical-care unit price thereof is relatively quite low compared to the case of using another apparatus. Therefore, expenditure becomes deficit, taking withdrawal of personnel expenses and expenses for apparatus etc. into consideration. According to the viewpoints of specialists with advanced knowledge, there are many cases where it is uncertain that the above-mentioned diseases of the circulatory system cannot be diagnosed based on the apexcardiogram provided using the mechanocardiogram recording device of FIG. 44 at present.

Moreover, from the viewpoint of medical education, even if teaching how to palpate correctly is desired, there is no available measuring means for displaying the motion of the heart palpated by an instructor objectively.

To improve such a problem, the inventor of the present invention has proposed a vital reaction recording device and a vital reaction recording method disclosed in Patent Document 1. The inventor of the present invention has created a prototype vital reaction recording device, actually measured a living body in attempt to diagnose the health condition. However, many problems to be solved for using them at medical sites are found.

Namely, since there has been no measuring device in practical use until now, there is a shortage of measured data for apexcardiogram useful for diagnosis. In addition, technology for diagnosing illnesses of circulatory organs based on the apexcardiogram measurement data has not been established yet, and thus cannot be used at medical sites.

Only the measuring device proposed by the inventor is capable of objectively displaying the motion of the left ventricle (henceforth, referred to as left ventricle), for which palpation has been attempted. However, how to utilize it has not been established yet.

From the viewpoint of medical education, it is necessary for medical students and residents to be able to study correct medical examination methods (notably, medical examination technology itself for palpation and an appropriate evaluation method for findings obtained through the medical examination). However, since usage of the measuring device proposed by the inventor has not been establishment yet, it cannot be used for teaching correct examination methods, and thus improvement in clinical medicine education is in demand.

### PRIOR ART DOCUMENTS

### Patent Documents

Patent Document 1: JP2008-113728

### Non-Patent Documents

Non-Patent Document 1: “Practice of examination of electrocardiogram and mechanocardiogram” issued by Japanese Association of Medical Technologists, Nov. 1, 1996, 2nd Printing, pp 212-222

### SUMMARY OF INVENTION

### Problems solved by the Invention

As mentioned above, use of an apexcardiogram for medical treatment is seldom anticipated at present. However, specialists recognize that palpation is important, and therefore a measuring device, which conducts highly reliable apexcardiogram and includes a processing method for processing the data obtained through the apexcardiogram and discovering disease examples of the circulatory system, is desired strongly.

The present invention is provided in view of the present condition, and one of the objects of the present invention is to further improve the vital reaction recording device proposed by the inventor of the present invention, provide a measuring device and an algorithm for data processing, which may be used at medical sites without measurement and experiment for a huge number of measured living bodies after a user has introduced the device, and provide a biometric information measuring method used for a biometric information measuring device and a biometric information measurement system, which are easily used at medical sites and accurately grasp the health condition of a living body.

Another object of the present invention is to provide a biometric information measuring method used for such a biometric information measuring device and a biometric information system at a low cost.

Yet another object of the present invention is to provide a biometric information measuring method used for a biometric information measuring device and a biometric information system, which may be widely used at nursing care sites as well as at hospitals having circulatory system medical practitioners, and also for health care of many human beings.

Still yet another object of the present invention is to provide at a low cost a biometric information measuring device and a biometric information measuring method, which allow highly accurate measurement and will greatly improve clinical medicine education by teaching medical students and intern doctors from the viewpoint of medical education. In addition, information acquired from the apexcardiogram is used to objectify the medical examination techniques, thereby making it possible to identify auscultatory sound (e.g., the third sound or the fourth sound), which greatly contributes to medical treatment (medical examination techniques, assessment of good or bad information, diagnosis, therapeutic effect assessment), and medical education.

### Solution to the Problems

A characteristic of the technical ideas according to the present invention invented for solving those problems is that a small-sized, lightweight, and inexpensive device is used, a sensor is placed on the chest or abdomen etc. of an living body to be measured, and a measuring device running based on an algorithm for recording change in pressure at several points near measuring areas and accurately displaying the health condition of the measured living body based on huge quantities of very complicated measurement data is provided.

In addition, a pressure sensor (a wave sensor such as an ultrasonic sensor, a lightwave sensor capable of detecting displacement and change in pressure of a subject to be measured, are referred to as pressure sensor collectively) is used as the above-mentioned sensor, thereby making it possible to conduct apexcardiogram and obtain biometric information that will complement apexcardiogram. Specifically, measuring change in pressure of the living body's surface, or propagating in the living body, waves, such as sonic waves (ultrasound is also referred to as sonic waves henceforth) or light waves and measuring the resulting echo may provide health information of at least the cardiac function of the living body with improved precision.

Embodiments according to the present invention are explained below concretely.

A biometric information measuring device and a biometric information measurement system, according to the present invention, measure and record at least one of heart beats, ascending-aorta beats, pulmonary artery (including at least one of truncus pulmonalis and pulmonary artery on the center side) beats, abdominal-aorta beats, and hepatic pulses, as at least one of apex beats, right ventricle heartbeats, and left atria heartbeats of a living body, and in addition to the heartbeat measurement, provide biometric information other than the same obtained through heartbeat measurement, such as measurement of ultrasonic echo or a reflected infrared ray.

To solve the problem, a first invention (invention 1 hereafter) as an example of the present invention is a biometric information measuring device for measuring and recording the heartbeat of a living body, the biometric information measuring device comprising: a pressure sensor (hereinafter referred to as a heartbeat sensor) for measuring motion or pressure of at least a part of the living body at which the heartbeat is to be detected, based on change in the position of the part or change in the pressure; a data processing means for extracting health information of a living body from a signal measured by the heartbeat sensor (the signal measured by the heartbeat sensor or an amplified signal of the signal are referred to as a heartbeat sensor output signal collectively hereafter); and a memory means for recording measurement data including the heartbeat sensor output signal, data processed by the data processing means, and temporary data generated during the processing; wherein the data processing means defines as a basic waveform, the heartbeat sensor output signal or the apexcardiographic waveform as one beat of measurement data, and defines as the basic waveform, the apexcardiographic waveform ranging from a predetermined time before the point (referred to as a QRS peak point hereafter) corresponding to each QRS positive vertex value (R) of II lead of standard 12 leads electrocardiogram for the same living body at the same time to the same predetermined time before the next QRS peak point; wherein the basic waveform is displayed with the horizontal and the vertical axis representing time and amplitude of the apexcardiographic waveform, respectively; if there is a minimum point (hereinafter referred to C1) on the apexcardiographic waveform within the range of 30 ms (millisecond) after and before a QRS peak point of the basic waveform, C1 is defined as a characteristic point P(2), if the C1 is not clear, the point corresponding to the QRS peak point on the apexcardiographic waveform is defined as the characteristic point P(2); a positive vertex on the apexcardiographic waveform between the QRS peak point and the point 160 ms before the QRS peak point (same hereafter) is defined as a characteristic point P(1); a positive vertex on the apexcardiographic waveform between the P(2) and the point 50 ms to 150 ms after the P(2) is defined as a characteristic point P(3); a positive vertex on the apexcardiographic waveform nearest to the 2A sound, which is an aortic-atresia sound on the phonocardiogram, between the 2A sound (II A sound) and the point less than 70 ms before the 2A sound is defined as a characteristic point P(5); regarding P(5), if the P(5) is a positive vertex nearest to the 2A sound on the phonocardiogram between the 2A sound and the point less than 40 ms before the 2A sound, that positive vertex is then defined as a characteristic point P′ (5); and if the P(5) is a positive vertex nearest to the 2A sound on the phonocardiogram between the 2A sound and the point less than 40 ms to 170 ms before the 2A sound, the positive vertex is defined as a characteristic point P″(5); if the P(5) is a positive vertex nearest to the 2A sound on the phonocardiogram between the 2A sound and the point 50 ms before the 2A sound, the positive vertex is defined as a characteristic point P″(5); if the P(5) is a positive vertex nearest to the 2A sound on the phonocardiogram between the 2A sound and the point 50 ms to 70 ms before the 2A sound, the positive vertex is defined as a characteristic point P″″(5) (the P(5), the P′(5), the P″(5), P′″(5) or P″″(5) are referred to as P(5) respectively or collectively, except for the case where the characteristic-points the P′(5), the P″(5), P′″(5) and P″″(5) are needed to be distinguished); a positive vertex on the apexcardiographic waveform between the point 150 ms after the P(2) and the point 70 ms before the 2A sound is defined as a characteristic point P(4); a negative extreme on the apexcardiographic waveform between the 2A sound and the point 50 to 150 ms after the 2A sound is defined as a characteristic point P(6);

if, between the 2A sound and 100 to 240 ms after the 2A sound, the P(6) exists, the positive vertex located after the P(6) on the apexcardiographic waveform is defined as a characteristic point P(7); the characteristic points P(1), P(2), P(3), P(4), P(5), P′(5), P″(5), P′″(5), P″″(5), P(6), and P(7) are defined as a first characteristic-point group; a waveform risen before the P(1), which is a positive vertex on the apexcardiogram, is defined as a wave A; a waveform risen from the P(2) and having a positive extreme on the apexcardiogram is defined as a wave E; and an ascending wave starting at the P(6) is defined as a wave F; a positive peak position of the wave A of the first-order differential waveform on the apexcardiogram is defined as a point a; a positive peak position of the wave E is defined as a point e; a positive peak position of the wave F is defined as a point f; and heights of the a, the e, and the f points are defined as a, e, and f, respectively; a first characteristic-point determination means is a determination means for determining whether there are at least two characteristic points in the first characteristic-point group; a second characteristic-point determination means is a determination means for defining ordinate value of the minimum position of the basic waveform as zero and determining height of at least one of the P(1), the P(2), and the P(7) when normalized, so as for the maximum coordinate value of the basic waveform to be 1000 points, a third characteristic-point determination means is a determination means for defining, regarding time of each of the characteristic points, P(2)-P(3) time (time from the P(2) to the P(3), same hereafter), ratio of P(3)-P(5) time to P(2)-P(6) time, P(6)-P(7) time, 2-P(6) time (time from the 2A sound to the P(6), same hereafter), and 2-P(7) time as characteristic factors, and determining the value of at least one of the factors; a first waveform determination means is a determination means for comparing to a waveform determination pattern stored in the biometric information measuring device or an apexcardiographic waveform determination pattern input to the biometric information measuring device from the outside, and determining the type of the basic waveform; a second waveform determination means is a determination means for determining the value of each of the a, the e, and the f; a third waveform determination means is a determination means for determining whether there is a section of the first-order differential waveform on the apexcardiogram running almost horizontally near a point having the first-order differential value of zero between the point e and the lowest point immediately before the point f; a fourth waveform determination means is a determination means for determining whether the lowest point immediately before the point f of the first-order differential waveform on the apexcardiogram falls within the first half of the section between the point having a first-order differential value of zero immediately before the lowest point and the point having a first-order differential value of zero immediately after the lowest point; a fifth waveform determination means is a determination means for determining whether the apexcardiogram includes a monopole graph not having P(5) but P(3), wherein if there is the monopole graph, whether the section after P(3) in time phase (referred to as P3 rear width hereafter), of the width of the graph at 700 points when the height of P(3) is normalized to 1000 points is less than 100 ms; and the data processing means comprises the first to the third characteristic-point determination means, at least one of the five determination means of the first to the fifth waveform determination means, and a health condition determination means for the living body to be measured. There is also provided an invention of a biometric information measuring system and a biometric information measuring method. Note that in the following description, description of a biometric information measuring device serves as description of a biometric information measuring system and description of a biometric information measuring method.

A second invention as an example of the present invention made by developing the Invention 1 (hereinafter referred to as Invention 2) is, in the biometric information measuring device as described in the Invention 1, the data processing means comprises at least the first characteristic-point determination means, and at least either the characteristic-point determination means or the waveform determination means.

A third invention as an example of the present invention made by developing the Invention 1 or Invention 2 (hereinafter referred to as Invention 3) is, in the biometric information measuring device as described in the Invention 1 or Invention 2, the data processing means determines regarding the P(3), the P(4), and the P(5) using the characteristic-point determination means and the waveform determination means, determines regarding the P(6) and P(7), and then determines regarding the P(1) and P(2), thereby assessing the health condition of an living body to be measured.

A fourth invention as an example of the present invention made by developing any one of the Inventions 1 to 3 (hereinafter referred to as Invention 4) is, in the biometric information measuring device as described in any one of the Inventions 1 to 3, the second characteristic-point determination means uses at least one of criteria: whether the P(1) is 300 points or less, whether the P(2) is 300 points or less, and whether the P(7) is 50 points or greater and 200 points or less.

A fifth invention as an example of the present invention made by developing any one of the Inventions 1 to 4 (hereinafter referred to as Invention 5) is, in the biometric information measuring device as described in any one of the Inventions 1 to 4, the third characteristic-point determination means uses at least one of criteria: whether either the P(2)-P(3) time is 50 ms or greater and less than 125 ms or falls between 125 ms and 150 ms, whether either the P(3)-P(5) time is not less than 45% of the P(2)-P(6) time, 40% or greater and less than 45%, or less than 40%, whether the P(6)-P(7) time is 100 ms, or 100 ms or greater and less than 150 ms, whether either the 2-P(6) time is less than 150 ms, or 150 ms or greater and less than 200 ms, and whether either the 2-P(7) time is less than 240 ms, or 240 ms or greater.

A sixth invention as an example of the present invention made by developing any one of the Inventions 1 to 5 (hereinafter referred to as Invention 6) is, in the biometric information measuring device as described in any one of the Inventions 1 to 5, information processing is carried out by defining that: possibility of severe left ventricular dysfunctions (systolic dysfunction and diastolic dysfunction) is low if there are the P(3) and the P(5) on the basic waveform, and if there is no P(5), left ventricular diastolic dysfunction is suggested, and/or left ventricular systolic dysfunction is suggested other than left ventricular diastolic dysfunction, and/or there is left ventricular hypertrophy and therefore determination as normal left ventricular contraction is suspected.

A seventh invention as an example of the present invention made by developing any one of the Inventions 1 to 6 (hereinafter referred to as Invention 7) is, in the biometric information measuring device as described in any one of the Inventions 1 to 6, information processing is carried out by defining that left ventricular systolic function is normal if the P3 rear width is less than 100 ms.

An eighth invention as an example of the present invention made by developing any one of the Inventions 1 to 7 (hereinafter referred to as Invention 8) is, in the biometric information measuring device as described in any one of the Inventions 1 to 7, information processing is carried out by defining that health condition of the living body is normal if the 2-P(6) time is less than 150 ms, otherwise, the health condition of the living body is abnormal if the 2-P(6) time is 150 ms or greater and less than 200 ms.

A ninth invention as an example of the present invention made by developing any one of the Inventions 1 to 8 (hereinafter referred to as Invention 9) is, in the biometric information measuring device as described in any one of the Inventions 1 to 8, information processing is carried out by defining that: the time interval between P(6) and P(7) is defined as a P(6)-P(7) time if there is the P(6); the health condition of the living body is normal if the P(6)-P(7) time is less than 100 ms; and the health condition of the living body is abnormal if the P(6)-P(7) time is 100 ms or greater and less than 150 ms.

A tenth invention as an example of the present invention made by developing any one of the Inventions 1 to 9 (hereinafter referred to as Invention 10) is, in the biometric information measuring device as described in any one of the Inventions 1 to 9, the second biometric information measuring device uses at least one of the criteria: regarding the a, the health condition is normal if the a is less than e/4; the health condition requires caution if the a is e/4 or greater and less than e/2; the health condition is abnormal if the a is e/2 or greater; and regarding the f, the health condition is normal if the f is less than e/2; the health condition requires caution if the f is e/2 or greater and less than 2e/3; and the health condition is abnormal if the f is 2e/3 or greater.

An eleventh invention as an example of the present invention made by developing any one of the Inventions 1 to 10 (hereinafter referred to as Invention 11) is, in the biometric information measuring device as described in any one of the Inventions 1 to 10, information processing is carried out by determining that: the health condition of the living body is normal if there is a section of the first-order differential waveform of the apexcardiogram running almost horizontally near the point having the first-order differential value of zero between the point e and the lowest point immediately before the point f, otherwise the health condition of the living body is abnormal if that section does not exist.

A twelveth invention as an example of the present invention made by developing any one of the Inventions 1 to 11 (hereinafter referred to as Invention 12) is, in the biometric information measuring device as described in any one of the Inventions 1 to 11, information processing is carried out by determining that the left ventricular diastolic function is normal if the lowest point immediately before the point f of the first-order differential waveform on the apexcardiogram falls within the first half of the section between the point having a first-order differential value of zero immediately before the lowest point and the point having a first-order differential value of zero immediately after the lowest point.

A thirteeth invention as an example of the present invention made by developing any one of the Inventions 1 to 12 (hereinafter referred to as Invention 13) is, in the biometric information measuring device as described in any one of the Inventions 1 to 12, If there is neither the P(3) nor the P(5), but there is the P(4), which is a positive extreme, at the point 150 ms or greater from the P(2), the health condition is determined to be abnormal, displaying to that effect.

A fourteenth invention as an example of the present invention made by developing any one of the Inventions 1 to 13 (hereinafter referred to as Invention 14) is, in the biometric information measuring device as described in any one of the Inventions 1 to 13, further comprising a means for determining whether the characteristic point has a predetermined range selected in time phase and height, and each measured data falls in the predetermined range, regarding at least one of the characteristic points the P(1) to the P(7) on the apexcardiogram and/or the characteristic points a, e, and f on the first-order differential waveform.

A fifteeth invention as an example of the present invention made by developing any one of the Inventions 1 to 14 (hereinafter referred to as Invention 15) is, in the biometric information measuring device as described in any one of the Inventions 1 to 14, further comprising: a means for modifying the criteria.

A sixteenth invention as an example of the present invention made by developing any one of the Inventions 1 to 15 (hereinafter referred to as Invention 16) is, in the biometric information measuring device as described in any one of the Inventions 1 to 15, further comprising a means for determining whether the characteristic point has a predetermined range selected in time phase and height, and each measured data falls in the predetermined range, regarding at least one of the characteristic points the P(1) to the P(7) on the apexcardiogram and/or the characteristic points a, e, and f on the first-order differential waveform.

A seventeenth invention as an example of the present invention made by developing any one of the Inventions 1 to 16 (hereinafter referred to as Invention 17) is, in the biometric information measuring device as described in any one of the Inventions 1 to 16, further comprising a means for inputting and setting predetermined ranges in time phase and height using a graphic symbol through an input part, such as a tablet, which is used by an examiner, regarding at least one of the characteristic points the P(1) to the P(7) on the apexcardiogram and/or the characteristic points a, e, and f on the first-order differential waveform, and a means for determining whether each measured data falls in the predetermined ranges.

An eighteenth invention as an example of the present invention made by developing any one of the Inventions 1 to 17 (hereinafter referred to as Invention 18) is, in the biometric information measuring device as described in any one of the Inventions 1 to 17, the wave sensor is an ultrasonic sensor, which receives ultrasound, and the biometric information measuring device comprises an ultrasound transmitter, an ultrasound receiver, and an ultrasonic echo analyzer.

A nineteenth invention as an example of the present invention made by developing any one of the Inventions 1 to 18 (hereinafter referred to as Invention 19) is, in the biometric information measuring device as described in any one of the Inventions 1 to 18, the wave sensor is an optical sensor, which receives a light wave, and the biometric information measuring device comprises a light wave transmitter and a light wave receiver.

A twentieth invention as an example of the present invention made by developing any one of the Inventions 1 to 19 (hereinafter referred to as Invention 20) is, in the biometric information measuring device as described in any one of the Inventions 1 to 19, further comprising a specific waveform elimination means for removing a specific waveform from waveforms contained in the measurement data representing the apexcardiogram, the specific waveform includes a first waveform appeared first after having started measurement or recorded the waveform, an abnormal waveform on electrocardiogram, a waveform at the beginning of respiration stop and the next waveform, a waveform at a time of respiration start and the waveform before the start, or a waveform including a larger noise than a predetermined size.

A twenty-first invention as an example of the present invention made by developing any one of the Inventions 1 to 20 (hereinafter referred to as Invention 21) is, in the biometric information measuring device as described in any one of the Inventions 1 to 20, further comprising a waveform classification means for classifying according to types, basic waveforms contained in the measurement data displayed as apexcardiogram, and a means for displaying or outputting the number of basic waveforms classified in each type.

A twenty-second invention as an example of the present invention made by developing any one of the Inventions 1 to 21 (hereinafter referred to as Invention 22) is, in the biometric information measuring device as described in any one of the Inventions 1 to 21, full waveform analysis of the basic waveform selected based on the basic waveform classification result by the waveform classification means, or input information from the outside of the data processing means is performed.

A twenty-third invention as an example of the present invention made by developing the Invention 22 (hereinafter referred to as Invention 23) is, in the biometric information measuring device as described in the Invention 22, the full waveform analysis is an analysis of the P(5) in the case where there is the P(3) and the P(5) does not exist out of the first characteristic points, or the waveform is not clear.

A twenty-fourth invention as an example of the present (hereinafter referred to as Invention 24) is a biometric information measuring system for measuring and recording the heartbeat of a living body, the biometric information measuring system comprising a measurement system for determination of the health condition of a living body to be measured, wherein the measurement system is constituted by using the heartbeat sensor, the data processing means, and the memory means, and also using a health condition determination method for the living body using the first to the third characteristic-point determination means, at least one of the five determination means of the first to the fifth waveform determination means, and a health condition determination means for the living body to be measured.

A twenty-fifth invention as an example of the present invention (hereinafter referred to as Invention 25) is a biometric information measuring system for measuring and recording the heartbeat of a living body, the biometric information measuring system using a heartbeat sensor for measuring motion or pressure of a part of the living body at which the heartbeat is to be detected, based on change in the position of the part or change in the pressure; wherein the heartbeat sensor comprises an ultrasound oscillation element and an ultrasound receiving element.

A twenty-sixth invention as an example of the present invention made by developing the Invention 25 (hereinafter referred to as Invention 26) is, in the biometric information measuring device as described in the Invention 25, an ultrasonic signal sent from the ultrasound oscillation element is a modulated signal obtained through amplitude modulation, frequency modulation, or phase modulation.

A twenty-seventh invention as an example of the present invention made by developing the Invention 25 or 26 (hereinafter referred to as Invention 27) is, in the biometric information measuring device as described in the Invention 25 or 26, the ultrasound receiving element is scanned and used.

A twenty-eighth invention as an example of the present (hereinafter referred to as Invention 28) is a biometric information measurement system for measuring and recording the heartbeat of a living body; the biometric information measuring system using a heartbeat sensor for measuring motion or pressure of a part of the living body at which the heartbeat is to be detected, based on change in the position of the part or change in the pressure; wherein the heartbeat sensor is a light wave receiver element.

A twenty-ninth invention as an example of the present (hereinafter referred to as Invention 29) is a biometric information measuring device for measuring and recording the heartbeat of a living body; the biometric information measuring device comprising: a pressure sensor (heartbeat sensor) for measuring motion or pressure of at least a part of the living body at which the heartbeat is to be detected, based on change in the position of the part or change in the pressure; an amplification means for amplifying the signal measured by the pressure sensor (the signal measured by the pressure sensor or the amplified signal of the signal are referred to as a sensor output signal collectively hereafter); a data processing means for extracting health information of the living body from the sensor output signal; and a memory means for recording the data processed by the data processing means or temporary data generated during the processing (hereinafter referred to as measurement data collectively); wherein the measurement data is data processed in sync with a specific electrocardiographic signal in time phase; the data processing means comprises: a means for identifying the sensor output signal for one beat or an apexcardiographic waveform from among pieces of the measurement data (hereafter referred to as a basic waveform identification means); a characteristic-point determination means and/or a waveform determination means of the basic waveform; and a health condition determination means for the living body to be measured; wherein the basic waveform identification means identifies as a basic waveform a section ranging from a predetermined time before a QRS peak point corresponding to each QRS positive vertex value (R) of II lead of standard 12 leads electrocardiogram to the same predetermined time before the next QRS peak point; the characteristic-point determination means and/or a waveform determination means of the basic waveform uses at least either the first characteristic-point determination means or the first waveform determination.

### Advantageous Effect of the Invention

The biometric information measuring method used for the biometric information measuring device and the biometric information measurement system, according to the present invention, allows measurment of apexcardiogram at bedside and even at daily life locales, thereby providing medically accurate health information of the heart of a living body to be measured, which is a great advantageous result.

Moreover, the present invention provides a measuring device, which allows the user of the device to obtain health information to some extent by himself/herself, and also provides a futuristic new health care system in which a measuring device terminal and a measuring device main body are connected to a radio communication system, for example, or a measuring device terminal is prepared for an information input unit of the measuring device main body, so as to input the measurement information to the measuring device main body and remotely or intensively manage health care of many living bodies. As a result, social health care will progress, which will provide extremely advantageous effects.

### BRIEF DESCRIPTION OF THE DRAWINGS

FIG. 1 is an explanatory drawing of a biometric information measuring device acdording to an embodiment of the present invention;

FIG. 2 is an explanatory drawing of measurement of apexcardiogram acdording to an embodiment of the present invention;

FIG. 3 is an example of apexcardiogram measured by a biometric information measuring device acdording to an embodiment of the present invention;

FIG. 4 is an example of apexcardiogram measured by a biometric information measuring device acdording to an embodiment of the present invention;

FIG. 5 is an example of apexcardiogram measured by a biometric information measuring device acdording to an embodiment of the present invention;

FIG. 6 is an example of apexcardiogram measured by a biometric information measuring device acdording to an embodiment of the present invention;

FIG. 7 is an example of apexcardiogram measured by a biometric information measuring device acdording to an embodiment of the present invention;

FIG. 8 is an example of apexcardiogram measured by a biometric information measuring device acdording to an embodiment of the present invention;

FIG. 9 is an example of apexcardiogram measured by a biometric information measuring device acdording to an embodiment of the present invention;

FIG. 10 is an example of apexcardiogram measured by a biometric information measuring device acdording to an embodiment of the present invention;

FIG. 11 is an example of apexcardiogram measured by a biometric information measuring device acdording to an embodiment of the present invention;

FIG. 12 is an example of apexcardiogram measured by a biometric information measuring device acdording to an embodiment of the present invention;

FIG. 13 is an explanatory drawing of a pressure sensor acdording to an embodiment of the present invention;

FIG. 14 is an explanatory drawing of a pressure sensor acdording to an embodiment of the present invention;

FIG. 15 is an example of apexcardiogram measured by a biometric information measuring device acdording to an embodiment of the present invention;

FIG. 16 is an example of apexcardiogram measured by a biometric information measuring device acdording to an embodiment of the present invention;

FIG. 17 is an example of apexcardiogram measured by a biometric information measuring device acdording to an embodiment of the present invention;

FIG. 18 is an example of apexcardiogram measured by a biometric information measuring device acdording to an embodiment of the present invention;

FIG. 19 is an example of apexcardiogram measured by a biometric information measuring device acdording to an embodiment of the present invention;

FIG. 20 is an example of apexcardiogram measured by a biometric information measuring device acdording to an embodiment of the present invention;

FIG. 21 is an example of apexcardiogram measured by a biometric information measuring device acdording to an embodiment of the present invention;

FIG. 22 is an example of apexcardiogram measured by a biometric information measuring device acdording to an embodiment of the present invention;

FIG. 23 is an example of apexcardiogram measured by a biometric information measuring device acdording to an embodiment of the present invention;

FIG. 24 is an example of apexcardiogram measured by a biometric information measuring device acdording to an embodiment of the present invention;

FIG. 25 is an example of apexcardiogram measured by a biometric information measuring device acdording to an embodiment of the present invention;

FIG. 26 is an example of apexcardiogram measured by a biometric information measuring device acdording to an embodiment of the present invention;

FIG. 27 is an example of apexcardiogram measured by a biometric information measuring device acdording to an embodiment of the present invention;

FIG. 28 is an example of apexcardiogram measured by a biometric information measuring device acdording to an embodiment of the present invention;

FIG. 29 is an example of apexcardiogram measured by a biometric information measuring device acdording to an embodiment of the present invention;

FIG. 30 is an example of apexcardiogram measured by a biometric information measuring device acdording to an embodiment of the present invention;

FIG. 31 is an example of apexcardiogram measured by a biometric information measuring device acdording to an embodiment of the present invention;

FIG. 32 is an example of apexcardiogram measured by a biometric information measuring device acdording to an embodiment of the present invention;

FIG. 33 is an example of apexcardiogram measured by a biometric information measuring device acdording to an embodiment of the present invention;

FIG. 34 is an example of apexcardiogram measured by a biometric information measuring device acdording to an embodiment of the present invention;

FIG. 35 illustrates the A wave;

FIG. 36 is an example of a tomogram obtained by a biometric information measuring device according to an embodiment of the present invention;

FIG. 37 is an example of apexcardiogram measured by a biometric information measuring device acdording to an embodiment of the present invention;

FIG. 38 is an example of apexcardiogram measured by a biometric information measuring device acdording to an embodiment of the present invention;

FIG. 39 is an example of apexcardiogram measured by a biometric information measuring device acdording to an embodiment of the present invention;

FIG. 40 is an example of apexcardiogram measured by a biometric information measuring device acdording to an embodiment of the present invention;

FIG. 41 is an example of apexcardiogram measured by a biometric information measuring device acdording to an embodiment of the present invention;

FIG. 42 is an example of apexcardiogram measured by a biometric information measuring device acdording to an embodiment of the present invention;

FIG. 43 is an example of apexcardiogram measured by a biometric information measuring device acdording to an embodiment of the present invention;

FIG. 44 is a photograph of the conventional mechanocardiogram recording device main body; and

FIG. 45 is a photograph of exemplified conventional various transducers.

### DESCRIPTION OF EMBODIMENTS

Next, embodiments according to the present invention are explained with reference to drawings. Note that each drawing used for explanation is schematically illustrated to such an extent that the embodiments of the present invention can be understood. For example, in a graph, a coordinate axis and the position of the graph may look slightly misaligned. For convenience of description of the present invention, a part of a drawing may be drawn with a different enlargement ratio, and there may be a case where a drawing used for explanation of an embodiment of the present invention may not always be similar to real things and/or what is described in the embodiment. Moreover, in each drawing, the same number is attached to the same structural components and items, thereby omitting overlapped explanation. Moreover, without notice, description of the biometric information measuring method may be shared with that of the biometric information measuring device as long as those skilled in the art can easily understand and no misunderstanding is likely to occur, and vice versa.

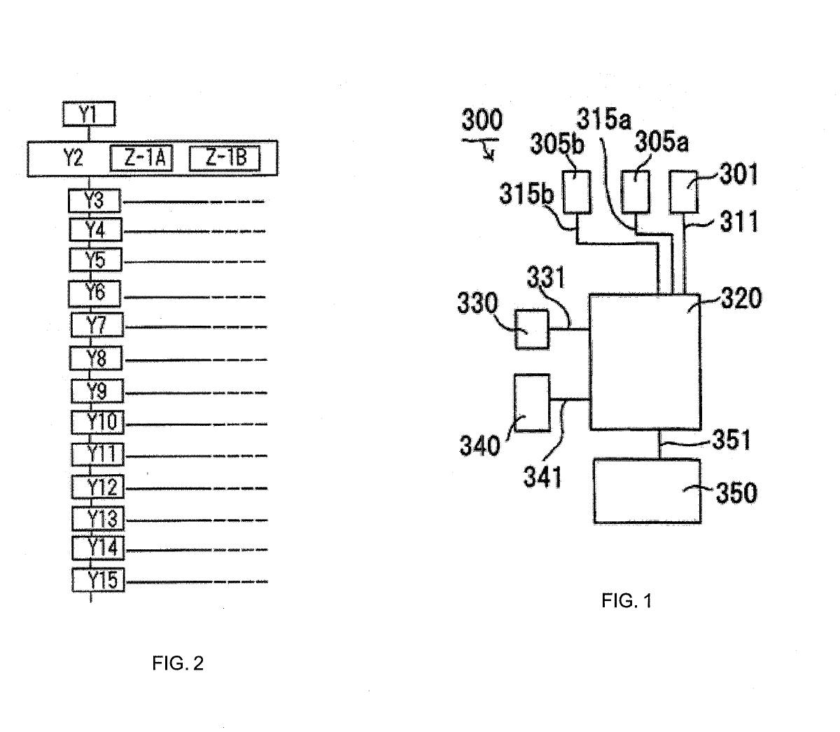

FIG. 1 is a block diagram explaining a biometric information measuring device of an embodiment according to the present invention, which is also capable of remote management.

In FIG. 1, a reference numeral 300 denotes a biometric information measuring device of the embodiment according to the present invention, 301 denotes a pressure sensor comprising a pressure sensor element or a pressure sensor unit as an apex beat sensor for measuring the cardiac apex beat (collectively referred to as a pressure sensor element hereafter), 305a denotes a cardiac sound sensor for measuring a heart sound, 305b denotes a sensor such as an electrocardiogram sensor for an electrocardiogram, 320 denotes a control/measurement data processing unit, 330 denotes a memory unit, 340 denotes a display unit, 350 denotes a remote management unit, 311 denotes a connection means for connecting between the pressure sensor 301 as a heartbeat sensor and the control/measurement data processing unit 320, 315a denotes a connection means for connecting between the sensor 305a and the control/measurement data processing unit 320, 315b denotes a connection means for connecting between the sensor 305b and the control/measurement data processing unit 320, 331 denotes a connection means for connecting between the memory unit 330 and the control/measurement data processing unit 320, 341 denotes a connection means for connecting between the display unit 340 and the control/measurement data processing unit 320, and 351 denotes a connection means for connecting between the remote management unit 350 and the control/measurement data processing unit 320. At least a part of the connection means may be constituted using a communication means such as a wireless communication means or an optical communication means.

In addition, the biometric information measuring device 300 may be constituted by a combination of several devices and/or units having respective functions mentioned above, thereby comprising a biometric information measurement system.

The pressure sensor 301 is a sensor element having a sensing element or a sensing part (hereafter referred to as a pressure sensor element) capable of detection of displacement in myocardium and/or hypoderm produced by the motion of a heart, or detection of pressure waves propagating the myocardium, hypoderm etc. as distinguishable data at several points. The pressure sensor element may be constituted by using, for example, a strain sensor, which changes in resistance etc. according to stress, a piezoelectric element constituted by a piezo-electric element and/or a semiconductor, an element which detects displacement of a part of a living body developed due to the pressure and/or stress of the apex of the heart using an electric means including an optical technology, an acoustic technology such as ultrasound, or a vibration technology etc. The pressure sensor element according to the present invention may also be an element capable of detection of a supersonic vibration signal, which is output from an ultrasound vibrator, then hits the heart, and reflects on the heart.

The pressure sensor element according to the present invention means one or a combination of those different sensors.

The cardiac sound sensor 305a is a sensor for measuring a cardiac sound, and may be a conventional cardiac sound sensor. Moreover, it may be integrated into the pressure sensor. The electrocardiogram sensor 305b is a sensor for measuring an electrocardiogram and a conventional electrocardiogram electrode may be used as a sensor.

In FIG. 1, the pressure sensor 301, the cardiac sound sensor 305a, and the electrocardiogram sensor 305b are deployed in measurement positions of a living body to be measured, respectively, detecting heartbeats, cardiac sounds, and signals for measuring an electrocardiogram, and loading the respective detected signals into the control/measurement data processing unit 320 via respective connection means mentioned above. The control/measurement data processing unit 320 then performs data processing to generate an apexcardiogram, a phonocardiogram, an electrocardiogram, etc. As a preferable example, the resulting apexcardiogram data and phonocardiogram data are extracted in sync with the electrocardiogram signal, then electrocardiogram data, phonocardiogram data and apexcardiogram data are stored, and displayed as a graph.

The data from the sensors is processed as data for apexcardiogram creation and other data, stored in the memory unit, and then displayed on the display unit if needed. The other data may be unnecessary, however, it may be a two-dimensional distribution of the sensed pressure strength, contact pressure etc. in a predetermined range of the chest.

In FIG. 1, the pressure sensor 301, the cardiac sound sensor 305a, the electrocardiogram sensor 305b, the control/measurement data processing unit 320, the memory unit 330, and the display unit 340 may constitute a biometric information measuring device or a biometric information measurement system. Moreover, at least a part of each of the control/measurement data processing unit 320, the memory unit 330, and the display unit 340 may be integrated into the remote management unit 350, thereby establishing a biometric information measuring device or a biometric information measurement system, which may be controlled remotely.

Moreover, the following embodiment is a preferable embodiment not narrowly limiting the present invention. It is constituted by: integrating into the pressure sensor 301a power supply part or a part equivalent to the power supply for driving the sensor etc. and a communication part etc. other than the pressure sensor element, structuring the pressure sensor 301 not wire connected to the biometric information measuring device main body, attaching the pressure sensor 301 to the chest of a living body to be measured, and sending the data of the apex beat measured by the pressure sensor element to the control/measurement data processing unit 320 in the biometric information measuring device main body through the communication part using a transmitting medium, such as an electric wave or light (including infrared rays and ultraviolet rays). With such a structure, measurement of the living body may be performed in an almost natural condition, and apexcardiogram may also be conducted remotely. This adds up to provision of a high quality apexcardiogram and more extensive health care, thereby allowing provision of an enhanced result of using the apexcardiogram according to the present invention.

According to the present invention, when the aforementioned pressure sensor attached to the living body to be measured has an important part of detecting the apex beat, such part is also referred to as a biometric information measuring device or a part for the biometric information measuring device.

Note that the electrocardiogram sensor and the phonocardiogram sensor may have a data acquisition mechanism or a communication function etc. and be deployed separately and independently from the biometric information measuring device main body, or otherwise may be connected to the biometric information measuring device main body via a cable. It is preferable that a transmission/reception mechanism is provided for an independent part for the biometric information measuring device main body, thereby allowing the biometric information measuring device main body to remotely control the part or the biometric information measuring device.

A necessary minimum part for data detection, such as apex beat data, required for health care of a living body to be measured is attached to a measuring position of the living body, and the measured data is then transmitted to the biometric information measuring device main body. The biometric information measuring device main body performs data processing of the transmitted data, and the result may be used for health management of the living body.

Apexcardiogram and corresponding data analyzing means, which are special features of the fundamental technical idea of the present invention, are described below in detail.

FIG. 2 is a block diagram explaining a measurement flow of apexcardiogram measuring device of the biometric information measuring device and an apexcardiogram measuring method according to an embodiment of the present invention. With this, the apexcardiogram measuring device is also explained substantially as an embodiment of the biometric information measuring device according to the present invention. The apexcardiogram measuring device may be constituted by only using this part, or the apexcardiogram measuring device may also be constituted using all of them. Moreover, the apexcardiogram measuring device may also be constituted by combining another means with that part or all of them.

In FIG. 2, reference numeral Y1 represents operations of turning on a power supply and pushing a start button while Y2 represents an operation of inputting information of a living body to be measured. This information denotes information including required information for measurement and diagnosis, and includes the following Z-1A and Z-1B, for example. In Y2, disease information of a living body to be measured is input in the following manner if needed.

Z-1A: Inputting by the living body to be measured or its surrogate;

The living body to be measured or its surrogate should input or file to ask an examiner to input. Required information, such as objective (medical treatment, health management etc), age, sex, body height, weight, blood pressure, and/or personal medical history, are input.

Z-1B: Inputting By a Specialist.

Information, such as whether there is a pacemaker, drug administration information, arrhythmia, and/or heart failure, is input.

In the case of a living body to be measured with a certain kind of abnormal electrocardiographic pattern, regardless of the original action of the left ventricular myocardium, the left ventricle systole may change, and therefore an electrocardiogram is investigated.

Y3 is a process of determining based on whether a pacemaker is embedded; wherein in this example, pacemaker-free living bodies are measuring targets. In the case of the living body to be measured in which a pacemaker is functioning, measurement should be performed according to a determination process for when a pacemaker is embedded.

Y4 is a process of diagnosing based on whether there is arrhythmia, which includes measuring objects except for atrial fibrillation with rapid ventricular response, ventricular tachycardia, ventricular fibrillation, paroxysmal supraventricular tachycardia, and advanced atrioventricular block, for example. For example, in the case of atrial fibrillation, apexcardiogram is conducted when the interval between the QRS wave measured at the same time as conducting apexcardiogram and the previous QRS wave is 800 msec or longer. Moreover, the cases of frequent premature contraction, bigeminy, trigeminy, and quadrigeminy are not subjected to regular measurement. It is preferable that cases where a constant regular pulse repeats at least 3 times or more are subjected to measurement. It is also preferable that a detection means for detecting items that are not subjects to be measured uses electrocardiogram analysis software, for example. However, there are cases where this does not apply depending on the data, and arrhythmic data may also be analyzed.

In the case of living bodies to be measured that have been determined not to be measuring objects in Y4, such as atrial fibrillation with rapid ventricular response, ventricular tachycardia, ventricular fibrillation, paroxysmal supraventricular tachycardia, and advanced atrioventricular block, measurement or analysis should be made according to a diagnostic process for predetermined cases.

Y5 is a diagnostic process based on whether there is a conduction disorder, which measures subjects except for complete left bundle branch block.

In the case of the living bodies to be measured with complete left bundle branch block, measurement or analysis is performed according to a predetermined diagnostic process for complete left bundle branch block.

In Y6, whether or not thoracotomy has been performed is input. If thoracotomy has been performed, measurement or analysis is then performed according to a predetermined diagnostic process for thoracotomy.

In Y7, data of administration of oral medicines and injection drugs is input.

In Y8, whether or not there is heart failure is input.

In Y9, measurement of apexcardiogram is started.

Measuring time is selectable and set up appropriately. For example, the measuring time may be chosen from 10, 15, and 20 seconds, allowing it to correspond to the respiration state etc. of the living body to be measured.

Measurement conditions are described below. Basically, measurement should be made with the living body to be measured in a left semi-lateral decubitus position. Although it is preferable to measure with the living body being relaxed in semi-exhalated phase, measurement should be made appropriately according to the condition of the living body to be measured. According to a measuring method with the living body not stopping respiration but quietly breathing, the base line of the apexcardiogram may shake, adding up to difficulty in diagnosis. If measurement is performed during deep inspiration (at a point of time when the living body has breathed out deeply), this easily leads to tense muscles and breath holding, resulting in difficulty in diagnosis. It is known that breath holding may change the blood pressure and the heart rate, resulting in a different condition than the resting state. In other words, it may enter an undesirable condition during deep inspiration. Therefore, many of the following data below are data obtained by measuring in a predetermined period of time with the living body in a relaxed state in the left semi-lateral decubitus position and with respiration being lightly stopped in mid-exhalated phase in principle.

The above-mentioned measurement conditions are particularly desirable measurement conditions. However, according to the present invention, it is found that apexcardiogram may be conducted effectively even while breathing, thereby allowing detection of the health information of the living body to be measured.

Apexcardiogram is conducted for a living body to be measured in, for example, an outpatient examination room and a general patient's room using the biometric information measuring device according to the present invention. In principle, the right upper extremity and the left lower extremity of a living body to be measured are respectively equipped with an electrocardiogram electrode; II lead of standard 12-lead electrocardiogram is conducted; a phonocardiogram microphone is attached near an intercostal space of the mid-position left sternal border in principle; the living body is placed in a left semi-lateral decubitus position, an examiner touches for the apex beat region and finds the strongest heartbeat position; a pressure sensor is applied as a heartbeat sensor to that region; and apexcardiogram is then conducted. All of the above operation, measurement, and recording can be easily performed by one examiner. Preparations for electrocardiogram, phonocardiogram, and apexcardiogram are carried out and they are then started.

In Y10 and subsequent steps, a part of the measurement process that has started in Y9 is extracted and explained.

In Y10, the signal measured by the pressure sensor of the biometric information measuring device according to the present invention is loaded into an electronic circuit, which then amplifies and removes noise from the measured signal.

According to a preferable embodiment of the present invention, measurement data is first loaded in sync with wave R of waves (waves QRS) of the electrocardiogram.

The measurement data includes at least three items: an electrocardiogram, a phonocardiogram, and an apexcardiogram.

To analyze the apexcardiogram, information of the electrocardiogram and the phonocardiogram is useful. For example, identification of wave R of the electrocardiogram is required, which may be carried out using a well-known method. Information of the first, the second, the third, and the fourth sound of the phonocardiogram and information of the carotid arterial pulse may also be useful. Such identification may also be performed using a well-known method.

Sensors for the electrocardiogram and/or the phonocardiogram in addition to the pressure sensor may also be arranged in the sensor for the apexcardiogram and then used. In this case, a greatly simplified operation may be provided, resulting in remarkable results. Moreover, use of a thermometer or an aerotonometer may provide better results.

As to the above-mentioned amplification and noise rejection, the order, the degree, the number of times thereof etc. may be set as needed or set automatically.

An amplification means used in Y10 may be able to utilize a conventional technology in the electronics or the optics fields effectively. The same applies to the noise rejection means. For example, an electromyography filter, a hum filter, and/or a drift elimination means may be used.

Noise removed in Y10 includes, for example, power supply noise, noise at the starting time of measurement, noise due to electric disturbance during measurement, noise during arrhythmia, noise emanating from whether there is respiration, such as expiration and inspiration, noise identified based on the examiner's know-how, noise pre-registered based on the examiner's know-how etc.

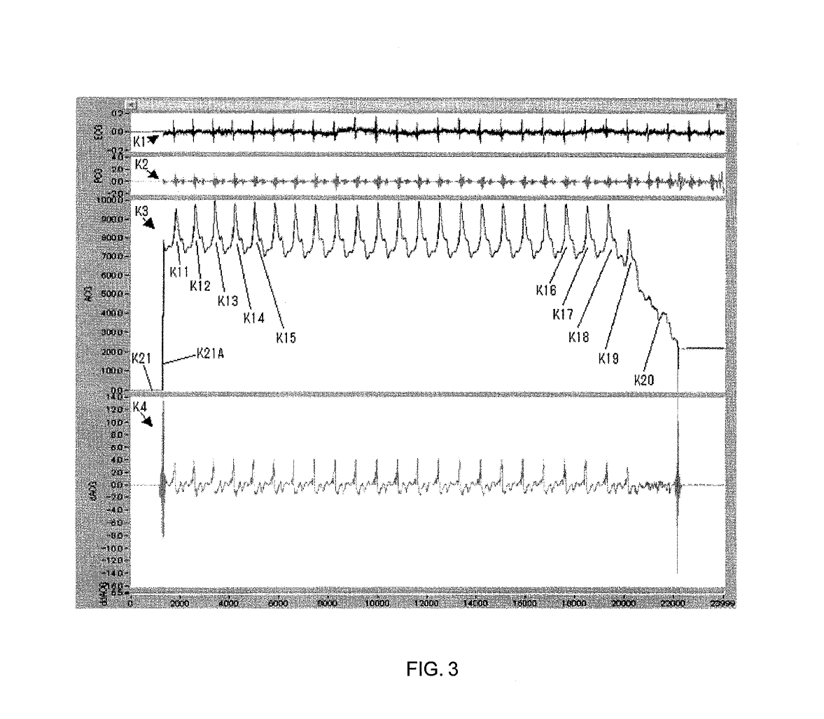

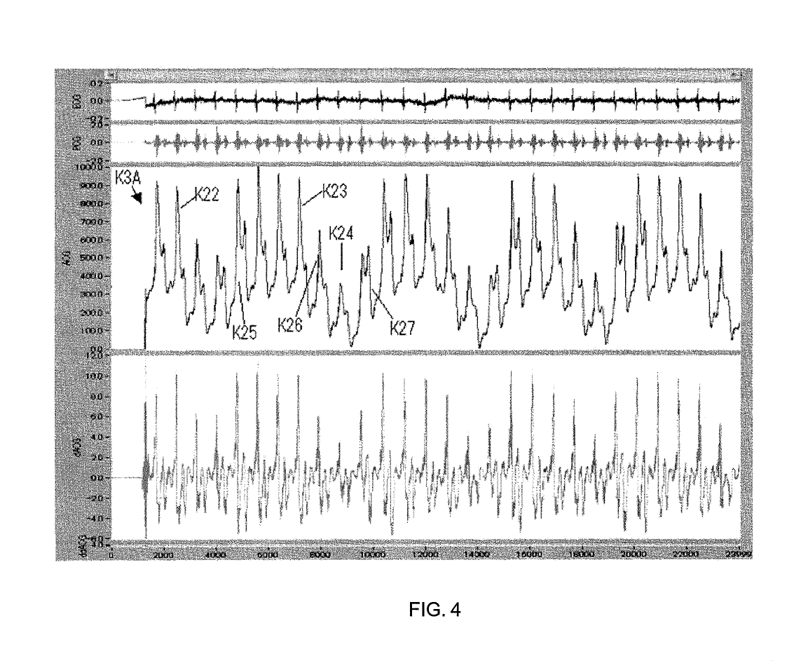

Note that the noise is described later, however, only a mere example thereof is explained below using FIG. 3. Namely, a rising edge K21A from a base line K21 and a basic waveform K19 immediately before the living body to be measured starts respiration midway are respectively recognized as a noise-contained waveform.

In Y11, the electrocardiographic signal is analyzed based on the above-mentioned measured signal, an abnormal item, such as arrhythmia, required for diagnosis is detected, and it is then displayed, attached with a flag, and subjected to a predetermined processing.

In Y12, all the basic waveforms are subjected to a predetermined feature analysis. This feature analysis result allows selection of a basic waveform useful for health information analysis of a living body to be measured and an invalid waveform, and also allows selection of a basic waveform to be subjected to health information analysis.

The biometric information measuring device, according to the present invention, includes an analysis means that carries out data-processing in Y9 to Y12, which is described later in detail.

The inventor of the present invention has studied in detail an example of measurement of approximately 1000 living bodies using the biometric information measuring device according to the present invention, and that study has allowed establishment of an analysis means for analyzing relevance between the apexcardiogram and pathological conditions with great accuracy. The analysis result according to the present invention is compared to information obtained by another means other than conducting apexcardiogram, such as MRI, myocardial scintigraphy, echocardiography, or cardiac catheter examination, information acquired by ocular inspection, palpation, and auscultation, and medical treatment and health information of a living body to be measured. As a result, it is learned that the health information of the living body obtained using the present invention has extremely high reliability.

Several methods may be used for the feature analysis of the basic waveform of the apexcardiogram using the biometric information measuring device according to the present invention.

First, identification of the basic waveform of the apexcardiogram, namely, extraction of the basic waveform is performed. To do this, an embodiment according to the present invention uses a method of identifying a basic waveform, which is defined as being located between a predetermined time before the QRS peak position and the next predetermined time before the next QRS peak position.

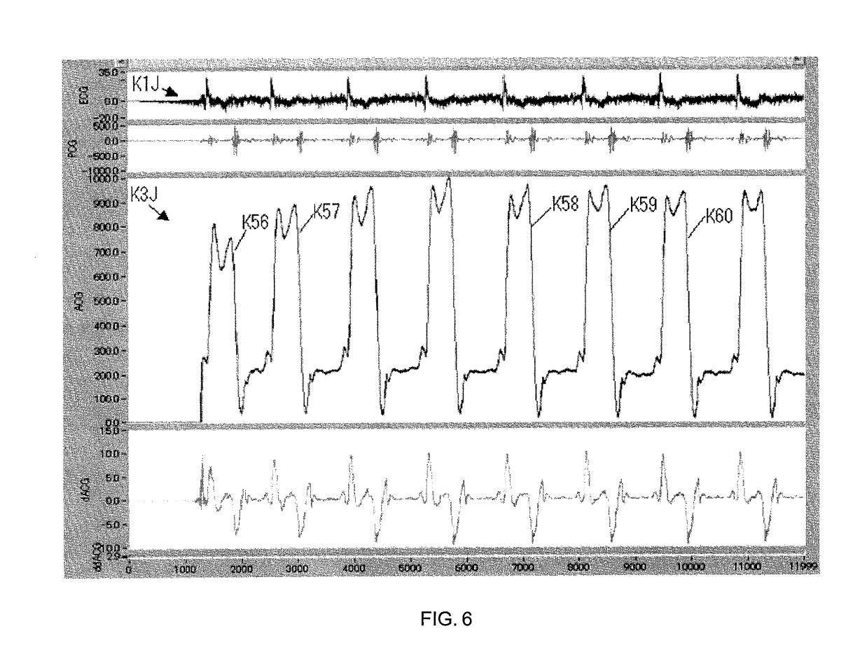

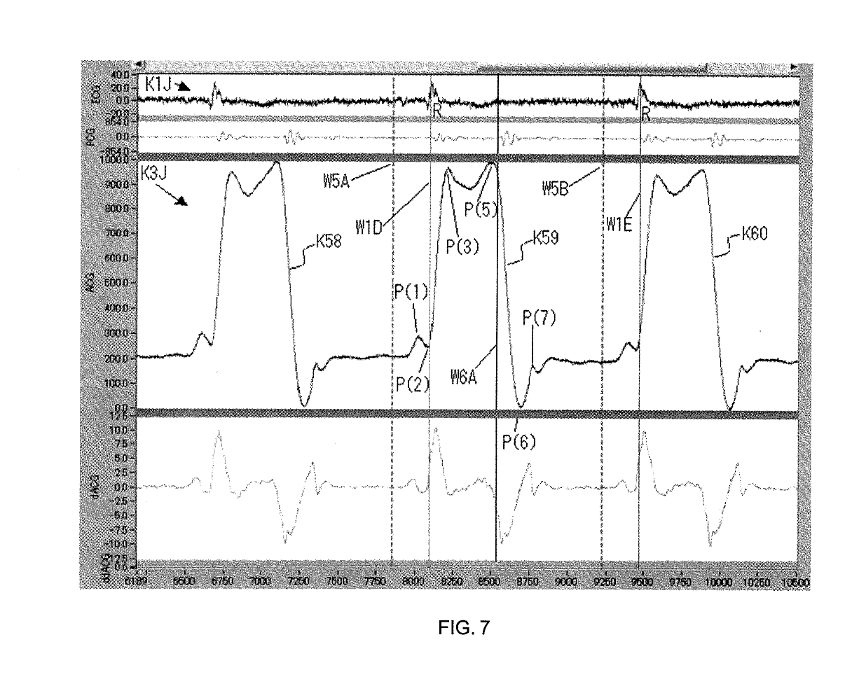

The identification method (extraction method) for the basic waveform is described later using FIGS. 6 and 7.

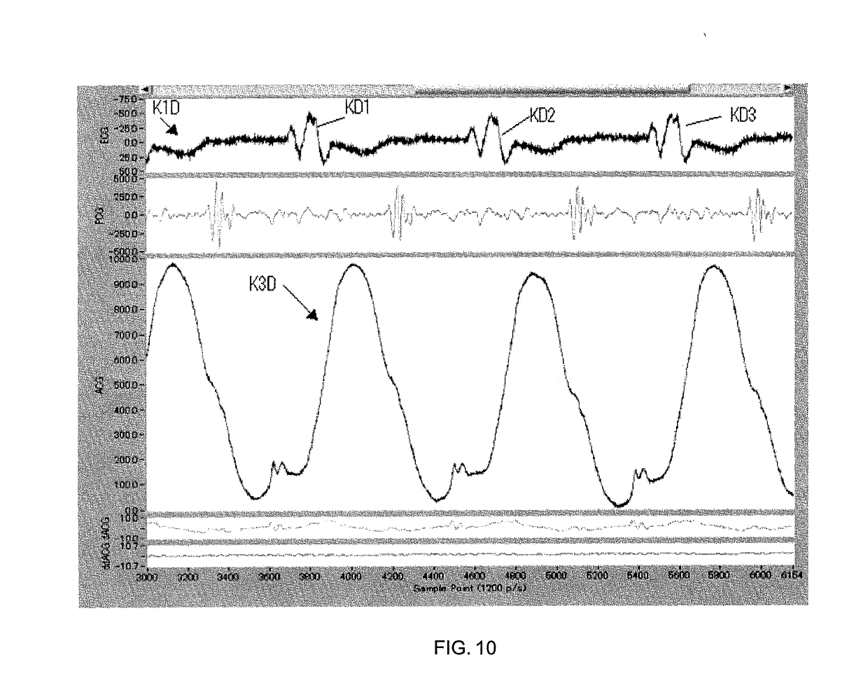

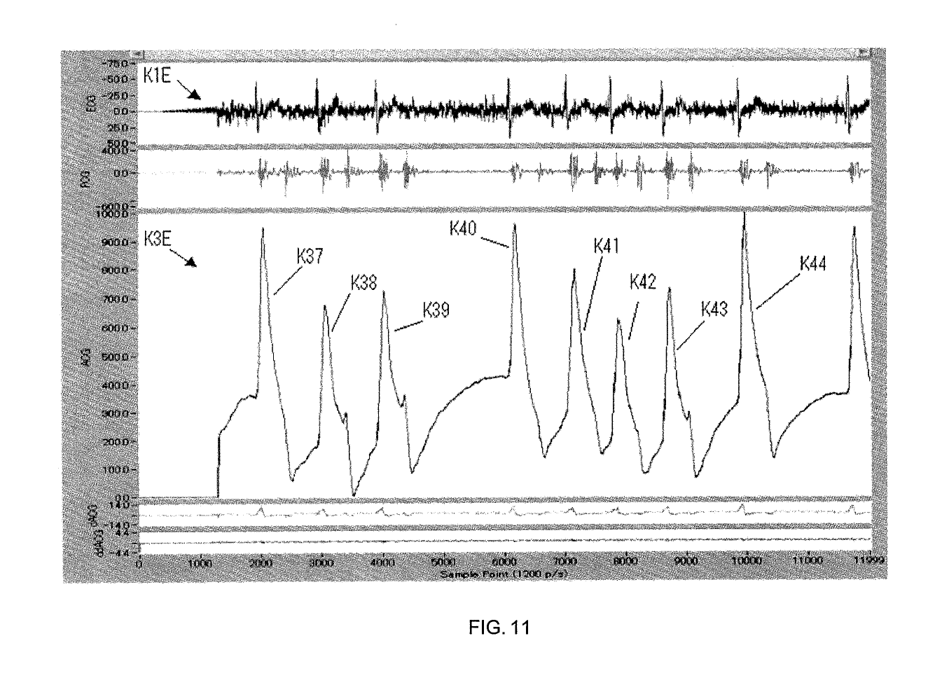

Next, in Y13 of FIG. 2, waveforms that should be excluded from the entire apexcardiogram of measured data in conformity with a predetermined standard determined considering the above-mentioned items and/or below-mentioned items are removed. The waveforms that should be excluded also include not only waveforms with problematic noise but also waveforms with arrhythmia, which are mentioned later using FIG. 9 and/or waveforms with conduction disorder etc., which are mentioned later using FIG. 10. If a specific waveform that should be excluded exists, the effect is then displayed, or a flag is attached thereto, and analysis required for that waveform etc. is performed according to a predetermined process defined independently.

In Y14 of FIG. 2, the type of each of the remaining basic waveforms is identified, and respective waveform types including what have been excluded, and the numbers thereof are displayed or recorded.

In Y15, selection of the waveform to be analyzed, analysis of measured data, extraction of health information, health management, various types of transmission etc. are performed. Namely, various kinds of processing, which are explained here with the present invention, are carried out according to the level of devices, purpose of use, symptoms etc.

The inventor of the present invention has conducted apexcardiogram according to the present invention for a living body to be measured, has conducted data analysis according to the present invention, and moreover to verify the results thereof, diagnoses obtained according to the conventional various measurement means other than the present invention and the inventor's medical knowledge have been considered collectively. As a result, the present invention of the biometric information measuring device having a characteristic-point analytic algorithm described below and the biometric information measuring method for the biometric information measurement system has been accomplished.

FIG. 3 shows an example of an electrocardiogram, a phonocardiogram, and an apexcardiogram for a living body measured for approximately 20 seconds using the biometric information measuring device according to the present invention; wherein they are an apexcardiogram obtained by amplifying the signals measured by the respective sensors and displaying in a predetermined display mode. One of orthogonal coordinate axes represents time, and the other axis represents intensity of sensor output signals. The time axis (hereinafter called horizontal axis) expresses one second divided into 1200 points, and the intensity axis (hereinafter called vertical axis) expresses minimum signal intensity of a displaying range of the apexcardiogram as normalized at zero point and maximum at 1000 points (Unless otherwise specified, the same applies to the apexcardiogram hereafter as a rule.)

In FIG. 3, reference numeral K1 denotes an electrocardiogram, such as II lead of standard 12-lead electrocardiogram (hereafter, if there is no necessity in particular, the same electrocardiographic waveform is illustrated, or otherwise if required, another electrocardiographic waveform, such as I lead of electrocardiographic waveform etc. may be used.) Reference numeral K2 denotes a phonocardiogram, and K3 denotes an apexcardiogram; both of which are displayed in sync with the electrocardiogram. Reference numeral K4 denotes the first-order differential waveform of the apexcardiogram K3. Reference numerals K11 to K19 denote basic waveforms contained in the apexcardiogram K3.

In FIG. 3, from the beginning of recording measurements, the apexcardiogram starts rising at the baseline K21, and at the position of a line K21A apexcardiogram data synchronized with the electrocardiogram rises up, and measured unit waveforms K11 to K18 are recorded successively while breathing is stopped. Judging the electrocardiogram and the phonocardiogram at the same time, the electrocardiogram shows no abnormality, while the phonocardiogram shows a respiratory signal after a position K20, and once the examined living body starts breathing midway during the unit waveform K19, change in the apexcardiogram is seen at K20.

FIG. 4 shows an example of an apexcardiogram obtained by measuring for approximately 20 seconds using the biometric information measuring device according to the present invention. The horizontal and the vertical axis are the same as those of FIG. 3.

In FIG. 4, K3A indicates apexcardiogram displayed in sync with the electrocardiogram. K22 to K27 denote apexcardiogram basic waveforms included in the apexcardiogram K3A. Each of the basic waveforms indicates characteristic points P(1) to P(7) which will be mentioned later, to have similarities, however, change in amplitude and waveform shape are found. Places of large amplitudes of the basic waveforms may be regarded as expiration, and small amplitudes may be regarded as inspiration. Inspiration begins from the end of the basic waveform K22 and ends at the beginning of the basic waveform K25, expiration is then carried out until the basic waveform K23, and inspiration is carried out between the basic waveforms 26 and 27, showing decrease in amplitude at the basic waveform 24. It is more preferable to process data during each analysis while taking these results into consideration.

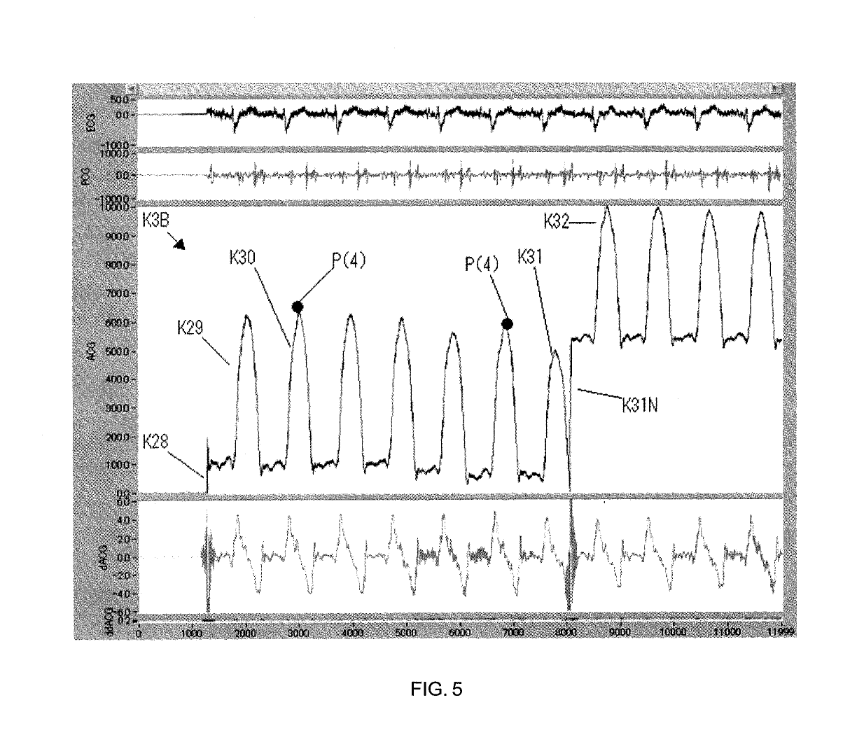

FIG. 5 shows an example of an apexcardiogram obtained by measuring for approximately 10 seconds using the biometric information measuring device according to the present invention. The horizontal and the vertical axis are the same as those of FIG. 3.

In FIG. 5, K3B indicates apexcardiogram displayed in sync with the electrocardiogram. K29 to K32 denote apexcardiogram basic waveforms included in the apexcardiogram K3B, and K28 denotes a line rising at the baseline. Each of the basic waveforms shows that characteristic point P(4) exists, and that neither characteristic points P(3) nor P(5) which will be mentioned later, exists. After immediate change indicated by K31N occurrs between the basic waveforms K31 and K32, similar basic waveforms start again from the basic waveform K32. This immediate change indicated by K31N is supposed to be noise.

FIGS. 6 and 7 show an example of an apexcardiogram obtained by measuring for approximately 10 seconds using the biometric information measuring device according to the present invention, explaining an example of how to extract a basic waveform and an example of the feature analysis. Note that FIGS. 6 and 7 show the same measurement data of the same measured living body (human being); wherein FIG. 7 has a greater enlargement ratio than that of FIG. 6. The horizontal and the vertical axis are the same as those of FIG. 3.

In FIGS. 6 and 7, reference numeral K3J represents an apexcardiogram displayed in sync with an electrocardiogram K1J. Reference numerals K56 to K60 are apexcardiogram basic waveforms included in the apexcardiogram K3J.

In FIG. 6, since basic waveform K56 is a waveform immediately after rising, it will be excluded from analysis targets, investigation of the remaining waveforms has made it clear that features and amplitude of those three waveforms continuing from waveforms K58 to K60 are similar to one another, and when there are such waveforms fitting the conditions, three continuous waveforms with larger amplitude and more similar waveforms should be chosen. It is preferable to choose an analysis target waveform in the manner explained below using FIG. 7.

In FIG. 7, reference numerals W1D and W1E denote lines indicating QRS peak positions, W5A and W5B denote lines indicating positions 200 ms before the QRS peak positions W1D and W1E, respectively, in time phase, and W6A denotes a line indicating the position of a 2A sound.

By setting the QRS peak positions as standards, an appropriate range of waveforms may be selected according to purpose.

To grasp the systolic state of the left atrium (also referred to as left fringe hereafter) correctly, it is preferable to acquire as much information as possible from several characteristic points.

To grasp the rising edge of the left atrial contraction wave (A wave) correctly, it is preferable to choose a range between the point 200 ms (milliseconds) before a QRS peak position and the point 200 ms before the next QRS peak position as an extracted range for the above-mentioned basic waveform of the apexcardiogram, according to the inventor of the present invention having investigated a large amount of measured data.

An analysis means of the basic waveform, which has been considered difficult to be analyzed until now because reliable data is seldom obtained, is explained next. The inventor has conducted apexcardiogram for many human beings including healthy persons and sick persons as living bodies to be measured using the biometric information measuring device according to the present invention, and has analyzed by comparing them to the health conditions of the measured human beings. This has leaded to findings that by defining characteristic points described below in detail and conducting analysis of a variety of data, health condition of the measured living body may be detected accurately, recorded, and displayed.

As a first characteristic-point determination means, the present invention is provides a characteristic-point analytic means for determining health information of the measured living body based on whether there are at least two of characteristic-points P(1), P(2), P(3), P(4), P(5), P′(5), P″(5), P′″(5), P″″(5), P(6), and P(7), as mentioned above.

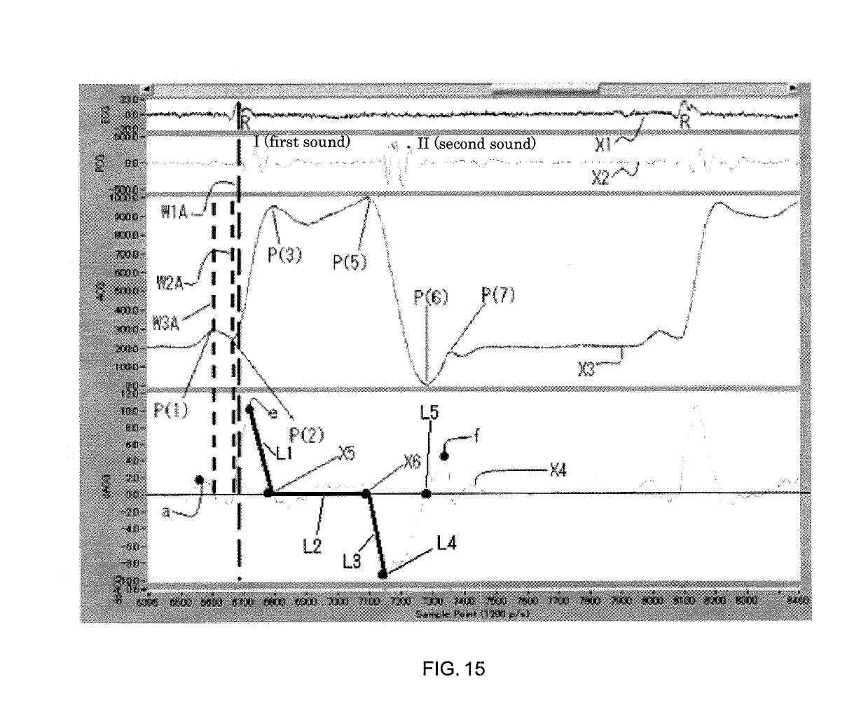

Regarding the above-mentioned P(5), different detection processing methods for healthy persons, which are explained using FIG. 15, for example, are required corresponding to the different cases: one where P(5) is P′(5), namely P(5) ranges between a 2A sound and the point 40 ms before the 2A sound on the phonocardiogram, and the other where P(5) is P″(5). Moreover, different detection processing methods for healthy persons, which are explained using FIG. 15, for example, are also required in the case where P (5) is P′″(5) and where P(5) is P″″(5).

As is described later in detail using an example, the results from analyzing the measurement data according to the present invention say that investigating whether there are at least two of the first characteristic points P(1) to P(7) and assessing a part of the health condition of the living body to be measured is possible.

The inventor has reached the conclusion resulting from full examination that analysis of the measurement data using the characteristic points P(1) to P(7) allows provision of highly reliable health information. However, there is fear that if characteristic points outside of the above-mentioned range are selected, reliability of the analysis may be impaired greatly.

Furthermore, the present invention provides as a second characteristic-point determination means a characteristic-point analysis means for determining amplitude of the characteristic points under specific conditions. For example, if there are P(1), P(2), P(3), P(5), P(6), and P(7), and the coordinate values of the basic waveform along the vertical axis are normalized where the coordinate value of P(6) or the lowest position of the basic waveform is set to zero and the coordinate value of the larger one of the coordinate values of P(3) and P(5) is set to 1000 points, a method for determining whether P(1) is 300 points or less, whether P(2) is 300 points or less, and whether P(7) is 200 points or less may be used as the second characteristic-point determination means. For example, if P(1) is 300 points or less and P(2) is also 300 points or less, health information to the effect that there is a high possibility that the left ventricular function in the end diastolic phase is normal may be displayed.

In the example of FIG. 7, P(5) is larger than P(3) in amplitude, that is, higher in coordinate value, in the basic waveform K59. When the lowest point P(6) is normalized to zero and P(5) to 1000 points, P(3) is 977 points high (coordinate value), P(1) is 277 points high, P(2) is 245 points high, and P(7) is 188 points high. This health information of the examined living body may be displayed that he or she has normal left ventricular systolic function.

If a waveform to be analyzed is K59 in FIG. 7, P(2) is 15 ms before line W1D, P(1) is 69 ms before line W1D, P(3) is 107 ms after P(2), P(5) is 36 ms before line W6A (namely, P(5) in this case is the aforementioned P′(5)), P(6) is 128 ms after line W6A, P(7) is 188 ms after line W6A, and all of the characteristic points P(1) to P(3) and P(5) to P(7) are within the aforementioned preferred range. The data measured and analyzed by the biometric information measuring device according to the present invention, indicates that the measured living body's left ventricle function is normal in the systolic state, and thus the health information of the living body to the effect that he/she is a healthy person in terms of the left ventricle function in the systolic state may be displayed. As a result from investigating other diagnoses of this living body collectively, it is proven that the above-mentioned health information is correct.

Furthermore, detailed study of many embodiments according to the present invention has concluded that further examination of the characteristic points P(1) to P(7) by the third characteristic-point determination means may provide reliable health information; where P(2)-P(3) time (period of time between P(2) and P(3), same hereafter), the ratio of P(3)-P(5) time to P(2)-P(6) time, P(6)-P(7) time, 2-P(6) time (period of time between the 2A sound to P(6); same hereafter), and 2-P(7) time are set as characteristic points (characteristic factors) may enhance reliability of the health information. According to the study of the present invention, P(2)-P(3) time of 50 ms or greater and less than 125 ms should be determined as normal, the same between 125 ms and 150 ms should be determined to require caution, and P(3)-P(5) time of 45% or greater of P(2)-P(6) time should be determined as normal, 40% or greater and less than 45% should be determined as a high possibility of being normal, less than 40% should be determined as abnormal, P(6)-P(7) time of less than 100 ms should be determined as a high possibility of being normal, 100 ms or greater and less than 150 ms should be determined as a high possibility of being abnormal, 2-P(6) time of less than 150 ms should be determined as a high possibility of being normal, 150 ms or more and less than 200 ms should be determined as a high possibility of being abnormal, 2-P(7) time of less than 240 ms should be determined as a high possibility of being normal, and 240 ms or greater should be determined as a high possibility of being abnormal.

In the example of FIG. 7, the P(2)-P(3) time is 107 ms, the ratio of the P(3)-P(5) time to the P(2)-P(6) time is 0.487 (namely 48.7%), the P(6)-P(7) time is 60 ms, the 2-P(6) time is 128 ms, the 2-P(7) time is 188 ms, which are within the aforementioned normal range.

Moreover, the first waveform determination means for comparing to a waveform determination pattern included in the device or a waveform determination pattern of an apexcardiogram input into the device from the outside and then identifying the type of the basic waveform may be used for analysis of the basic waveform.

Furthermore, the second waveform determination means for analyzing using the first-order differential waveform of the apexcardiogram may be used for analysis of the basic waveform.

The second waveform determination means determines and processes the following items and may also be used as a waveform determination means; where wave A has a waveform having a positive vertex of P(1) in the first-order differential waveform (dACG) of the apexcardiogram in FIG. 7, for example, and rising from the apexcardiogram before and after the positive vertex, wave E is a left ventricle systole wave having a positive extreme point P(3) and precipitously rising from point P(2) of the apexcardiogram, and wave F is a precipitously rising wave starting at the P(6), having positive vertex values: points a, e, and f (assuming that height of points a, e, and f are represented by a, e, and f, respectively). According to the study of those points a, e, and f, information processing may be performed as follows as a waveform determination means. That is, regarding a, if a is less than e/4, health condition is determined as normal, if a is e/4 or greater and less than e/2, the health condition is determined to require caution, and if a is e/2 or greater, the health condition is determined as abnormal, and regarding f, if f is less than e/2, the health condition is determined as normal, if f is e/2 or greater and less than 2e/3, the health condition is determined to require caution, and if f is 2e/3 or greater, the health condition is determined as abnormal.

In the example of FIG. 7, the points a, e, and f are 1.36, 10.22, and 4.61 in height, respectively, and a/e=0.13, and f/e=0.45; therefore, the health information of the living body may be displayed to the effect that he/she is a healthy person in terms of the left ventricle systolic function.

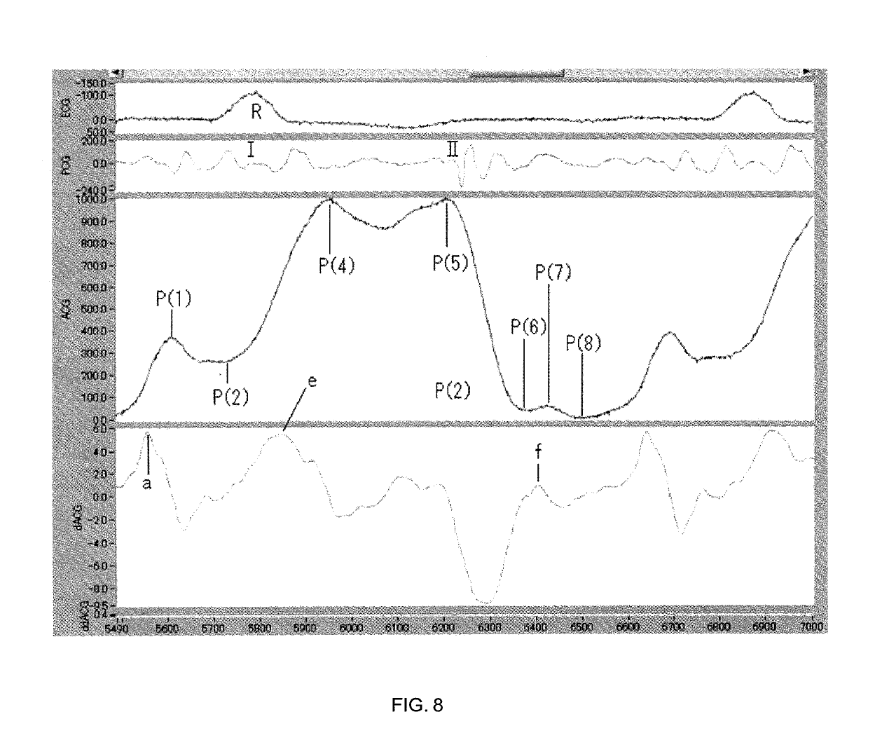

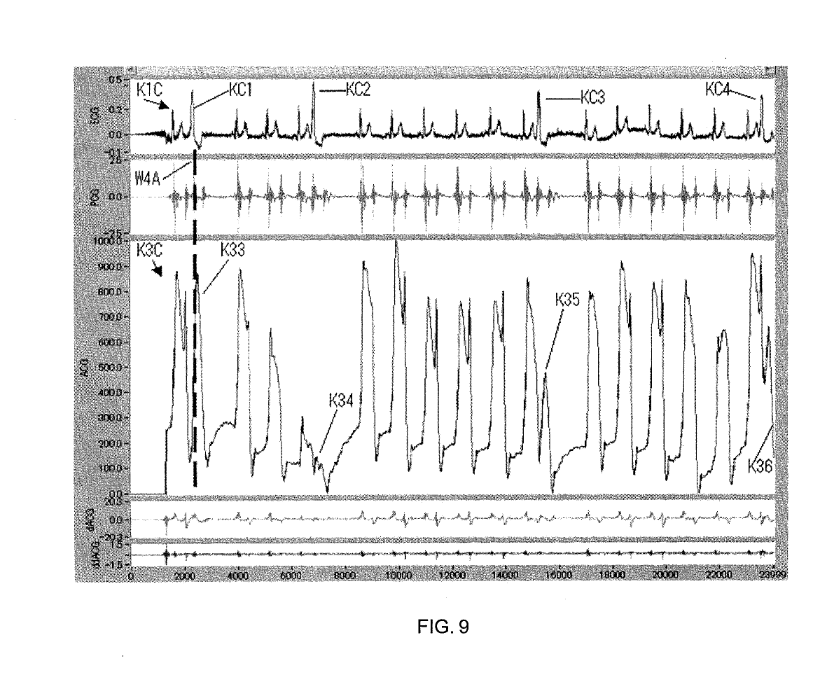

FIG. 8 shows an example of the apexcardiogram measured using the biometric information measuring device according to the present invention, thereby explaining an example of the case where the lowest point P(8) of a basic waveform has developed after the characteristic point P(6) of a basic waveform. The horizontal axis and vertical axis are the same as in the case of FIG. 3. As is seen in FIG. 8, P(7) is generated after P(6), and the lowest point P(8) is generated afterward. Moreover, there is no P(3), but there are P(4) and P(5). FIG. 8 illustrates a case where the left ventricular function is abnormal. Moreover, although not illustrated, P(8) may appear twice, and should also preferably be determined as abnormal.

Moreover, the graph may not be flat between P(7) and the next P(1), but may rise gradually. This case should also preferably be assessed as abnormal.

The first, the second, and the third characteristic-point determination means, and the first and the second waveform determination means may be used independently, or may otherwise be used in combination thereof. In many cases, the health condition of a living body to be measured may be diagnosed much more accurately using them in combination.

FIG. 9 shows an example of the apexcardiogram measured for approximately 20 seconds using the biometric information measuring device according to the present invention. The horizontal axis and vertical axis are the same as in the case of FIG. 3.

In FIG. 9, reference numeral K1C represents an electrocardiogram and K3C represents an apexcardiogram displayed in sync with the electrocardiogram. Arrhythmia is observed at positions KC1 to KC4 when waveforms of the electrocardiogram are analyzed by using a software for analyzing electrocardiographic waveforms. When waveforms of the apexcardiogram corresponding to the arrhythmia are analyzed by using a waveform analysis software according to the present invention, abnormality in the basic waveforms of the apexcardiogram K3C indicated by K33 to K36 is detected. Accordingly, predetermined flags are added to the measurement data, and analysis etc. progressed by proceeding to a separately defined, predetermined procedure.

FIG. 10 shows an example of a partially enlarged apexcardiogram measured using the biometric information measuring device according to the present invention; wherein reference numerals KM and K3D denote an electrocardiogram and an apexcardiogram, respectively, which are displayed in sync with the electrocardiogram. Analysis of the waveforms of KD1 to KD3 of the electrocardiogram using an electrocardiographic wave analysis software program etc., has diagnosed a complete left bundle branch block, which is a typical case of conduction disorder. In this case, analysis etc. is then performed in conformity with a predetermined procedure.

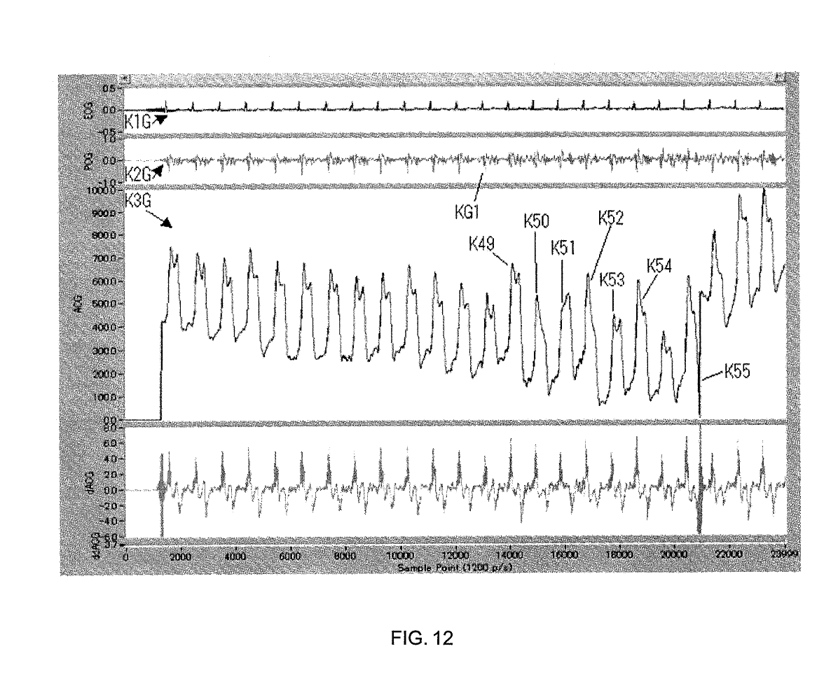

FIG. 11 shows an example of the apexcardiogram measured for approximately 10 seconds using the biometric information measuring device according to the present invention.

In FIG. 11, reference numerals K1E and K3E are an electrocardiogram and an apexcardiogram, respectively, which are displayed in sync with the electrocardiogram. K37 to K44 are respectively basic waveforms of the apexcardiogram. Analysis of the apexcardiographic waveform using the wave-analysis software program according to the present invention has clarified that it includes two types: one that has the characteristic points P(3) and P(5), such as waveform K38, and the other that has the characteristic point P(3), such as waveform K40, but no P(5), and that intervals of the basic waveforms have changed drastically. This analysis has also clarified that the living body suffers from atrial fibrillation, which is a type of arrhythmia, and in this case, proceeding to an independently predetermined processing where analysis etc. is performed.

Use of both the electrocardiographic analysis software program and the phonocardiographic analysis software program for analysis of the apexcardiogram allows provision of much more reliable health information than analysis of only data of the apexcardiogram.

According to the embodiment of the present invention, the apexcardiograms shown in FIGS. 9 to 11 are cases of requiring analysis through an independently predetermined analysis means other than the ordinary analysis. To respond to such a case, data of a measured living body whose condition of disease is known beforehand is taken, a feature map (e.g., a matching table of data and medical condition), a waveform determination pattern etc. are created, for example, and analysis is conducted using a predetermined analysis software program so that health conditions can be classified accurately.

FIG. 12 shows an example of the apexcardiogram measured for approximately 20 seconds using the biometric information measuring device according to the present invention.

In FIG. 12, reference numeral K3G denotes an apexcardiogram, which is displayed in sync with the electrocardiogram K1G. According to an analysis made using the data analysis software program according to the present invention, reference numerals K49 to K54 denote basic waveforms included in the apexcardiogram K3G, and K55 denotes a jump in waveform due to noise. K2G denotes a phonocardiogram. Each basic waveform rises and falls like a big drifting wave, and it looks like there are three waveform types: K49, K50, and K51, in which the majority is K49. It seems that the living body to be measured starts respiration at or around the waveform K49, and since the respiration moves the apex, the basic waveforms after the waveform K50 must be affected by the respiration. In analysis, waveforms up to the waveform immediately before the basic waveform K49 except for the waveform immediately after the rising edge of the apexcardiogram K3G are chosen to be analyzed.

According to the present invention, a variable factor, such as respiration information, is detected, data correction is made through, for example, compensation of the variable factor, so as to remove as much of the variable factor as possible, and basic waveforms are then extracted, thereby allowing analysis of the apexcardiogram.

Next, an example of measurement data processing by the biometric information measuring device according to an embodiment of the present invention is explained.

Data measured by a pressure sensor or another sensor is taken at a sampling rate of 12000 points per 10 seconds, for example, and the sampled data is then stored in memory and subjected to noise elimination and/or amplification.

In light of change in the detected output from the pressure sensor due to the position of the pressure sensor and/or how it is attached, data should be normalized with the maximum pulsating amplitude being set to 1000, which makes it easier to evaluate the data. Note that there are two examples of the measured apexcardiogram according to the present invention: one that one waveform is normalized and displayed, and the other that multiple wave forms are normalized and displayed with one of them being centrally displayed.

The amplified data is stored in memory and input into a waveform/graphic processor, which generates a graph if data is for apexcardiogram, electrocardiogram, and phonocardiogram, graphic processing is performed if data is for amplitude distribution and/or intensity distribution of heartbeats, and a feature extraction processing is carried out using those processing results. For example, extreme values are extracted from the graph of the apexcardiogram, the extreme values are compared, and extreme values in the first and the second order differential waveform and a waveform of the apexcardiogram are extracted through the feature extraction processing.

The data subjected to the feature extraction processing is stored as detected data in memory, and evaluation of the extracted waveforms, the feature etc. is conducted. As a result of evaluating whether the graph and/or diagram obtained through the waveform/graphic processing is appropriate, if it is determined to be inappropriate, or otherwise, change in a processing condition is demanded for research etc., new or modified amplification and noise elimination conditions and/or new or modified feature extraction conditions will be set up, so as to perform data-processing.

If the extracted waveforms, the feature etc. are determined to be appropriate, the first determination or evaluation of the health information is conducted, and detected data, such as biometric information, is created. In addition to using other information than that of the presently obtained measurement data, such as blood pressure or drug dosage information, the second determination or evaluation of the health information may be carried out.

If further examination is determined as necessary in each processing, additional determination and evaluation are then carried out in a predetermined data processing step.

Moreover, the above-mentioned data processing, determination, and evaluation may be carried out automatically or semi-automatically in conformity with a software program etc., they may be carried out for medical specialist's diagnosis and evaluation, or they may be carried out in combination

If a problem with creation of the detected data such as biometric information etc. is determined, through the first and the second determination or evaluation, waveform/graphic conditions etc. are then set up, the waveform/graphic processing is performed, and health information is created in a processing condition setting step.

Change-over-time information of the detected data for the same living body, statistical data that may be useful as information for diagnosis, a model pattern of the apexcardiogram etc. are stored in a reference data storing unit in memory, thereby providing more objective, accurate diagnosis.

The second determination information is stored in memory, displayed on a display device if needed, and health information of the living body is created. The health information of the living body is displayed on the display device if needed.

What should be displayed on the display device is not limited to such information, and if required, apexcardiogram, health information etc. may be displayed on the display device whenever necessary. Moreover, the display screen may have a configuration capable of being divided into multiple areas, and different pieces of data may be displayed at the same time thereon. Such a configuration allows all results of multiple analysis steps to be displayed and viewed. Such a configuration also allows a user of the device or the device itself to determine quickly and correctly.

The apexcardiogram itself is naturally important information for feature extraction and determination of the apexcardiogram. In addition, differential data of the apexcardiogram also plays an important role, as is apparent from the embodiment according to the present invention. For example, in the case where feature extraction is carried out according to a software program, evaluation of features is carried out, an automatic or a semiautomatic determination is carried out, and/or an apexcardiogram difficult to determine is interpreted by a medical practitioner, the first- and the second-order differential data makes it easier to carry out the feature extraction and evaluation, and many diagnoses in many cases.

There are many possible variations of the measuring method and the data processing method according to the embodiment of the present invention. For example, the apexcardiographic graph obtained though the graphic processing will change as the noise elimination conditions are modified. When what processing conditions should be set to will greatly influence determination accuracy of the device. The embodiment of the biometric information measuring device according to the present invention includes further limited processing conditions as a part of the invention.

Regarding data for an electrocardiogram and a phonocardiogram, the biometric information measuring device may be designed to receive and use, as externally input data, the measurement data that may be in sync with an apexcardiogram and is provided by an independent measuring means from the biometric information measuring device according to the embodiment of the present invention. This allows miniaturization of the device and low cost.

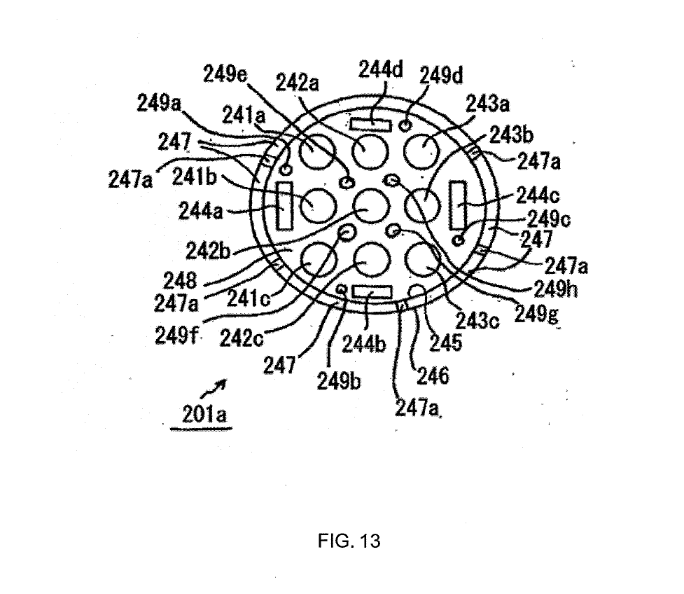

FIG. 13 views and explains an example of a pressure sensor functioning as a heartbeat sensor of the biometric information measuring device, according to the present invention, for measuring heartbeats; wherein the sensor has a circular contour.

In FIG. 13, reference numeral 201a denotes a pressure sensor; each of 241a to 241c, 242a to 242c, and 243a to 243c denotes a pressure sensor element for detection of the heartbeat of a living body; each of 244a to 244d denotes a sensor unit for detection of the contact pressure, etc. between the pressure sensor and a living body; 248 denotes a semiconductor board; 245 denotes the inner wall of a pressure sensor periphery; 246 denotes the outer wall of the pressure sensor periphery; 247 denotes a space between the outer wall 246 and the inner wall 245; 247a denotes a partition unit for partitioning the space 247; 249a to 249d denote fixing point correction means; and each of 249e to 249h denotes a microphone, which may also be used as a cardiac sound sensor.

In FIG. 13, each of the pressure sensors is a piezoelectric element formed in the semiconductor board 248.

The pressure sensor 201a is connected to a control/measurement data processing unit via wiring as in FIG. 1, or using radio or optical transmission and reception etc., thereby sending data measured by each sensor to the control/measurement data processing unit, and measuring in response to an instruction sent from the control/measurement data processing unit if needed, sending the resulting measured data to the control/measurement data processing unit, or allowing reception thereof by the fixing point correction means or the like.

The sensor units 244a to 244d for detecting the contact pressure etc. on a living body are capable of detection of the contact pressure on the living body, and may have a means for changing the relative position of the pressure sensor 201a to the living body, such as the fixing point correction means 249a to 249d, in response to an instruction from, for example, the control/measurement data processing unit if needed for the purpose of using the biometric information measuring device.

The space 247 may be partitioned into multiple areas: one that may be used for suction effect, and the other that may be used for pressurizing.

Pressure in the space 247 may be changed, so as to be utilized for attachment and detachment of the pressure sensor 201a to/from the living body if necessary for the purpose of using the biometric information measuring device. For example, in the case where the space 247 is divided into multiple areas by the partition unit 247a of FIG. 13, attachment and detachment of the pressure sensor 201a to/from the living body may be carried out, and/or change of the relative position of the pressure sensor 201a to the living body may be carried out, by changing the pressure in each of the partitioned areas.

Microphones themselves may be deployed in the illustrated positions for the microphones 249e to 249h, or otherwise those positions may be used as sound collecting holes, so as to deploy a sound detecting element inside or on the back side of the board 248. Such deployment allows miniaturization and enhancement of sound detecting characteristics.

While the number of the sensor units for detection of the heartbeat of a living body may be changed for the purpose of using the biometric information measuring device, three or more is preferable, and are notably arranged in two dimensions. Furthermore, there are cases where the sensor units 244a to 244d for detection of contact pressure etc. with the living body are not deployed depending on the purpose of using the biometric information measuring device.

Even though the pressure sensor 201a is constituted without arranging some of or all of the fixing point correction means 249a to 49d, the microphones 249e to 249h, and the space 247, it may be used as a pressure sensor for heartbeat detection.

The pressure sensor 201a in FIG. 13 is an example of the sensor units for detection of a living body's heartbeat, which are arranged in a two-dimensional 3×3 matrix, and is capable of measurement of change in pressure on the living body at every sensor unit position such that the pressure change can be distinguished among those positions, thereby detecting the living body's heartbeat correctly. Moreover, a measuring system configured so as to measure change in pressure on the living body at every sensor unit position simultaneously, or measure the same as a video image allows observation in an equivalent or a higher level than palpation of the heart, namely, allowing a medical examination with extremely high reliability.

The number of the two-dimensional sensor units or the number of detecting elements and how they are arranged are not restricted to the aforementioned number and arrangement. With the sensor units constituted in a 3×3, a 5×5, and a 7×7 matrix, a comparatively smaller number of sensor units capable of grasping actual motion of the heart may be available at a low cost. Aside from a matrix arrangement, they may be arranged in a concentric circular pattern or a radial pattern so as to improve the detection precision. From the viewpoint of operability and detection precision etc., a certain number of the censor units and an arrangement thereof allowing detection of the center thereof are preferable.

The pressure sensor in FIG. 13, according to the embodiment of the present invention, is not narrowly limited thereto. The number of pressure sensor elements arranged two-dimensionally may be m×n, for example, where m and n denote an integer, respectively, and pressure sensor elements or pressure detecting units in a 5×9 or 9×15 matrix may be arranged in an elliptical form with 1-cm short axis and 3-cm long axis, and/or they may be configured to be able to fit between human ribs, for example, thereby measuring with sufficient precision the heartbeat of the human beings whose ribs are pronounced, and providing many variations thereof. A two-dimensional arrangement of the pressure sensor elements or pressure sensor parts of the pressure sensor, according to the present invention, will provide extremely advantageous effects, namely not only conducting apexcardiogram but also correctly grasping the complicated motion of the heart, which includes a twisting movement, thereby providing correct diagnosis.

Many pressure sensor elements may comprise an extremely large number of sensor units by taking advantage of lithography and semiconductor technology.

Although not illustrated, the well-known element MEMS may also be used as the pressure sensor for detection of pressure changes at multiple points.

Another suitable example of the pressure sensor may be a strain sensor arranged on a flexible substrate as a pressure sensor element. An example of the strain sensor may be an element whose electrical resistance changes according to stress, for example.

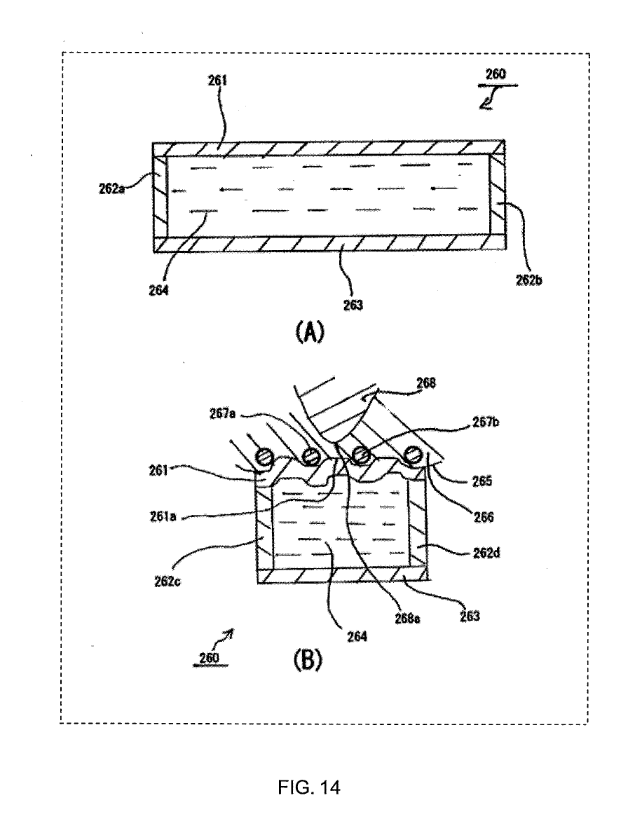

FIG. 14 is a diagram explaining a pressure sensor as a heartbeat sensor, according to am embodiment of the present invention; wherein (A) is a cross-sectional view of the pressure sensor cut along the length, and (B) is a cross-sectional view of the pressure sensor applied to the chest of a living body to be measured (human being) cut along a line perpendicular to the ribs. Reference numeral 260 denotes a pressure sensor, 261 denotes a pressure sensor element comprising the pressure sensor, 262a to 262d denote the side walls of the pressure sensor, 263 denotes the back wall of the pressure sensor, 264 denotes a liquid, 265 denotes chest wall epidermis of the living body to be measured, 266 denotes chest wall hypodermis of the living body, 267a and 267b denote ribs of the living body; 268 denotes the left ventricle of the living body, and 268a denotes the apex of the living body. Although not illustrated, at least one of the side walls 262a to 262d and the back wall 263 has a communicating unit or a pressure regulation unit for pressurization or decompression of the liquid 264. A pressure sensor element 261 is flexible and will change the pressure of the liquid 264, for example, to deform itself.

The back wall 263 has a rigid body in this embodiment. With such a structure, user-friendliness at the time of measurement while controlling the shape of the pressure sensor element may be improved.

When an examiner applies the pressure sensor element 261 of the pressure sensor 260 to the position of the chest wall epidermis 265 of a living body subjected to measurement of apexcardiogram, it adheres thereto, and pressurizes the liquid 264, a portion 261a in FIG. 14 (B) pressurizes the epidermis 265 between the ribs 267a and 267b, so as for the epidermis to dent, thereby allowing correct detection of the motion of the apex 268a with high sensitivity.

The portion of the living body to which the pressure sensor is applied is shaped into a three dimensionally round form, so as for the living body not to feel pain.

The main part of the pressure sensor element 261 has three rows on a flexible resin sheet as a board, each being made up of ten strain detector elements deployed independently electrically, which plays a role as a pressure sensor element, and it also has only one row of the same where each of the strain detector elements has electrical resistance that changes according to stress. Although not narrowly limited to this configuration, each of the elements is electrically wired in the same manner as is widely done with the liquid crystal display device having m×n picture elements (m and n are integers), and thus change in electric characteristics of the respective elements that change according to motion of the apex, which is transmitted to the respective elements, may be detected. Dimensions of the main part of each element (not including the wiring) are 0.2×0.7 (mm), wherein the 0.7 mm is a dimension of each element along the row of ten elements.

As a pressure sensor element, other than the strain detector element, piezoelectric elements using a piezoelectric material and stress detector using semiconductor elements etc., each having an m×n matrix, are fabricated as prototypes.

Preferable values of m and n as in the m×n matrix are one or greater and three or greater, respectively. A notably preferable value of n is ten or greater.

Dimensions of the portion of the pressure sensor 260 adhered to the living body to be measured are 5 mm×15 mm where m=1 and n=18. According to a more preferable example, dimensions of the portion of the pressure sensor 260 adhered to the living body to be measured should be 30 mm×60 mm, and fabricating a pressure sensor of m=3 and n=70. These dimensions allow easier measurement from the viewpoint of operability.

Moreover, according to a notably preferable example, dimensions of the portion of the pressure sensor 260 adhered to the living body to be measured should be 60 mm×60 mm. A 15×15 matrix of the pressure sensor elements is deployed in a 10 mm×10 mm inner region of that portion with high density, fewer than those elements are deployed in the region ranging from the contour of the 10 mm×10 mm region to that of an outer 30 mm×30 mm region with medium density, and even fewer are deployed in even outer region with low density. With this pressure sensor, it is possible to recognize the complicated motion of the left ventricle as well as the expansion thereof, and therefore it is possible to obtain information, which used to be determined through palpation conventionally, as recordable data. As a result, apexcardiogram may be conducted very precisely.

The majority of apexcardiograms exemplified in the embodiments according to the present invention and FIGS. 6, 7, and 15 except for the example of the apexcardiogram given using ultrasound are provided and displayed using the pressure sensor constituted by a piezoelectric element, with the portion of the pressure sensor adhered to the living body to be measured being 5 mm×13 mm in dimensions, where m=1 and n=15.

The present invention is not limited to the liquid 264. It may alternatively be a gas or something that can deform the pressure sensor element 261 may be used.

Moreover, the liquid 264 may be omitted, and thus it can be made thinner.

While the pressure sensor 260 may be applied to the chest wall by the examiner's hand, it may alternatively be applied to the chest wall with a band etc. Moreover, a structure of fixing the pressure sensor 260 to the chest wall by suction may be provided.

Moreover, the pressure sensor 260 may be constituted in a sheet-like shape. This may alleviate a sense of discomfort at the time of attaching to the living body to be measured, and improve adhesion to the chest wall, thereby providing an apexcardiogram that is closer to the actual state.

A portable biometric information measuring device may be attained by integrating a power supply, an arithmetic device, a transmission/reception device etc. into the pressure sensor 260.

As mentioned above, it is preferable to use the pressure sensor not only for carrying out pressure sensing of the heartbeat itself, but also directing correction of the position of the pressure sensor to move it to an appropriate position. In this way, the pressure sensor that is easy to use and can measure correctly is attained.

Since the peripheral form of the pressure sensor is very important for practical measurement and the pressure sensor should usually be applied to the skin of the living body to be measured by applying some force, it is preferable that the pressure sensor has a peripheral form not causing the living body any distress, such as pain. When the sensor is round, elliptical or a similar form, the living body to be measured does not have any pain even if the sensor is pressed against the living body strongly, allowing further accurate measurement. Use of a flexible pressure sensor for the same reason or for the purpose of improving the measurement precision lessens deterioration of measurement precision due to the shape of the chest wall of the human being with pronounced ribs etc. Furthermore, in the case of using not the round form but a polygonal form, such as a quadrangle or a form having a greater number of sides, it is preferable to round the corners thereof.

When the sensor has a polygonal form, such as a quadrangle or a form having a greater number of sides, it is convenient to provide an additional function.

Various forms of the pressure sensor may be chosen according to the kind and the form of the pressure sensor element, objective of measurement, the health condition of the living body to be measured etc.

Heartbeat measurement may be adversely affected by mixed noises. Naturally, it is useful to select and use a conventional noise suppression/elimination means, which has been utilized in electronic engineering.

Since a measuring object for the pressure sensor for detecting displacement and/or pressure change, according to the present invention, is a living body, specially considered conditions are required, as is well-known in the signal amplification, noise processing etc. However, different forms of elements utilized in various fields, such as the piezoelectric element utilizing the piezoelectric phenomenon or the electrostatic pressure sensor utilizing capacitance change between electrodes, may be used by adjusting themselves to meet necessary medical conditions. Therefore the technology for many pressure sensor elements or displacement detection elements may be utilized. As a result, a pressure sensor that is inexpensive, reliable, and suitable for medical practice may be provided

While the apexcardiogram is said as offering many kinds of information regarding the health conditions of a living body to be measured, no biometric information of living body can be obtained conventionally with the living body as is in its natural state, as mentioned above. That is, the conventional measurement is made even with a big problem or high measurement cost while the living body to be measured must be left in a tense state or must require special attention in a restrained environment, such as a soundproof room. Therefore, it is considered to be extremely low in usefulness for medical treatment.

According to a preferable embodiment for measurement of apexcardiogram of the present invention, at least one ultrasound sending element and ultrasound receiving element are provided to the pressure sensor; wherein an ultrasound wave sent from an oscillation element hits and reflects on the apex of the heart, and is then received by a receiving element, which then conducts apexcardiogram. The oscillation element and the receiving element are of a type of m=1 and n=240, respectively, each of twelve-element groups outputs at the same time in turn one after another by being scanned. Regarding each oscillation element and each receiving element, the same element may be used as the oscillation element and the receiving element in the following manner. That is, time-sharing between oscillation and reception is carried out, so as to prepare a time for the same element to function as oscillation element and a different time for the same element to function as the receiving element so that the ultrasound wave from the oscillation element is reflected on the apex and then received by the same element, thereby conducting apexcardiogram.

Moreover, according to a preferable embodiment of the present invention, an ultrasound sending element and an ultrasound receiving element are provided as a pressure sensor. The ultrasound sending element sends only ultrasound waves of a single type of wavelength from the same oscillation element. The ultrasound wave sent from the oscillation element hits and reflects off of the apex, received by an ultrasound receiving unit, which is either made up of multiple receiving elements deployed adjacent to one another, each being able to receive independently, or has a reception unit, and apexcardiogram is then conducted. In the case of using multiple kinds of ultrasound waves as a heartbeat measurement wave, it is preferable to use an independent ultrasound oscillation element for each kind. Such a configuration of each oscillation element outputting only ultrasound waves of a single type of wavelength will provide a synchronized ultrasound phase, thereby providing much a clearer apexcardiogram.

To conduct apexcardiogram using ultrasound, many technologies in the electronic engineering field may be used to frequency-modulate, amplitude-modulate, or phase-modulate an ultrasound wave and then amplify the resulting modulated signal into a reference signal.

There was no thinking of using an ultrasound wave for apexcardiogram conventionally. Use of the conventional ultrasonic echo measuring device together allows more accurate examination of the cardiac function, thereby verifying the assessed health condition through apexcardiogram and/or carrying out further extensive examination.

These ways may be applied to the case of using a light signal as the pressure sensor, so as to conduct apexcardiogram. Furthermore, in the case of using the light signal, technology of continuously outputting a very narrow-width pulse in a femtosecond order and making dual-photon absorption and multiphoton absorption phenomenon occur in the living body may be utilized to further extend practical use of the device.

The present invention not only has solved the big problem that measurement is costly, but also has made it possible to obtain biometric information, with the living body being in a nearly natural state.

As is apparent from each embodiment, the apexcardiogram as an example of biometric information recorded according to the present invention offers many kinds of information regarding the health condition of the living body to be measured.

It is important to extract required information for health condition management from this biometric information appropriately. However, as long as there is no available algorithm for detecting and utilizing that information appropriately, the information cannot regretfully be useful for the health condition management. The inventor of this application has measured and fully examined how this information can be extracted and utilized, and has found an algorithm for biometric information extraction, where use thereof has lead to development of the biometric information measurement system and the biometric information measuring device for accurate diagnosis of the health condition of the living body to be measured from the technical viewpoint.

Note that a wave sensor element, such as an ultrasonic sensor element, an optical sensor element etc., is used as a pressure sensor element in the biometric information measuring device according to the present invention, to measure the inner condition of the living body, such as motion of the heart itself, blood flow, or blood components, conduct apexcardiogram, and obtain supplementary information to the apexcardiogram information so that reliability of the health information of the living body will be improved, and health care of the living body will be performed with high reliability.

For example, in FIG. 13, an ultrasound sending element and an ultrasound receiving element are deployed in a sensor unit, the ultrasound receiving element receives an ultrasound signal that is emitted from the ultrasound sending element to the inside of the living body, reflected and returned back from there, the received data is then analyzed by an electric circuit of the control/measurement data processing unit in FIG. 1, so that an apexcardiogram described later and health information may be obtained. Moreover, a light signal sending unit and a receiving unit are deployed in the sensor unit, receiving a laser light that has been sent and returned back, and the electric circuit of the control/measurement data processing unit analyzes the laser light, providing biometric information. Although each pressure sensor element may be deployed as an independent sensor, multiple kinds of sensors may also be deployed with them.

In the case of the photodetector element and the ultrasonic sensor element in FIGS. 13 and 14, for example, the range of detection may be enlarged, so as to change the angle of incidence to the living body, and thus precision may be improved.

Below, conducting apexcardiogram according to the present invention is mainly explained based on the embodiment in detail again.

FIG. 15 shows an example of an apexcardiogram of the living body to be measured (human being) who has normal left ventricular systolic function measured by using the living body information measuring device according to an embodiment of the present invention. The first sound, the second sound and the like in a phonocardiogram are illustrated in FIG. 15 and may be illustrated in other Figures. Reference numeral X1 of FIG. 15 denotes an electrocardiogram, X2 denotes a phonocardiogram, X3 denotes a curve representing an apexcardiogram, X4 denotes a first-order differential curve resulting from differentiating curve X3 of the apexcardiogram as the horizontal axis by time, W1A denotes a line indicating a QRS peak position, W2A denotes a line indicating the position of the characteristic point P(2), and W3A denotes a line indicating the position of the characteristic point P(1).

Characteristic values in the apexcardiographic waveforms for analyzing measured data are described using FIG. 15. First, a positive wave due to left atrial contraction was confirmed. This is wave A, and its positive extreme point is P(1). If wave A has no positive extreme, continuously rises and reaches P(2) as described later, P(2) is regarded as P(1). P(1) was confirmed almost simultaneously with the fourth sound, and existed before the wave QRS. A rising point P(2) was generated next. This was confirmed at the starting point of a left ventricular systolic wave (wave E) and near the vertex of the electrocardiogram QRS wave. Wave E precipitously rose from P(2), forming the positive extreme point P(3) in an early systolic phase. P(3) almost coincided with the left ventricular ejection starting point. From P(2) to P(3) corresponded to the isometric diastolic phase. It then slowly declined, and re-ascended to a late systolic phase, reaching the positive extreme point P(5). P(5) existed just before the 2A sound. P(5) was the left ventricular systole starting point. The 2A sound generated immediately after P(5), and nosedived into P(6). P(6) existed in the early diastolic phase, becoming the lowest point (negative extreme) of the apexcardiogram. It existed in a little later time phase than the vicinity of the mitral opening point. A relatively pricipitous rising wave from P(6) was confirmed. This rising wave is defined as a rapid inflow wave (wave F). The vertex thereof is P(7). Wave F generated as a result of rapid blood inflow from the left atrial in the left ventricular early diastolic phase to the left ventricle. The end of wave F almost coincides with the time phase of the third sound. Subsequently, a gradual rising wave was confirmed due to slow blood inflow from the left atrial in the left ventricular mid diastolic phase to the left ventricle, leading to wave A.

The positive peak values of wave A, wave E, and wave F of the first-order differential waveform X4 in the apexcardiogram are defined as points a, e, and f, respectively. X5 and X6 coincide with P(3) and P(5), respectively, in time phase as the differential value is zero in each case.

While the apexcardiogram of FIG. 15 is a graph in which each point is displayed relatively clearly, a graph having characteristic points P(1), P(2), P(3), P(5), P(6), and P(7) that may look obscure depending on the living body to be measured or dimensions and/or form of a sensor may be measured. Even in such a case, medical professionals may be able to identify each point from their on-site medical experience etc. Applying an expert system using a medical specialist-specific determination method to the apexcardiogram measuring device as an embodiment of the biometric information measuring device according to the present invention, and in addition, letting medical professionals read and diagnose the practical measurement data taken by the apexcardiogram measuring device according to the present invention will greatly improve the results of the present invention.

Regarding a selected waveform, a waveform is defined within a range from the point immediately before P(1) to the point immediately before the next P(1). Of the waveforms provided through measurement, a waveform with the peak magnitude should be selected in principle, and normalized, with the peak magnitude being defined as 1000 points, and then measured. Note that it is necessary to make sure that the waveform is smooth and there is no abnormal vibration mixed therein. To specify P(2) first, a waveform when P(2) of the waveform to be measured is located almost in the same position as P(2) of the immediately previous waveform (less than 50 points when normalized) should be selected. Alternatively, there may be a case where multiple continuous waveforms within an arbitrary section, which are measured and stored, are normalized and used for measurement. Moreover, there may be another case where use of multiple discontinuous waveforms is also effective for correct determination of data. The horizontal axis of the drawing is displayed with a unit of 1 defined by dividing 10 seconds by 12000. In the case of apexcardiogram, the vertical axis is displayed with a unit defined by dividing the maximum value and the minimum value of nearly one waveform by 1000, respectively, as a rule, and for electrocardiogram and phonocardiogram it is a relative value as a standard.

FIG. 15 shows an example of a 21-year-old male living body to be measured having a normal left ventricular function. A process of analysis is described using FIG. 15.

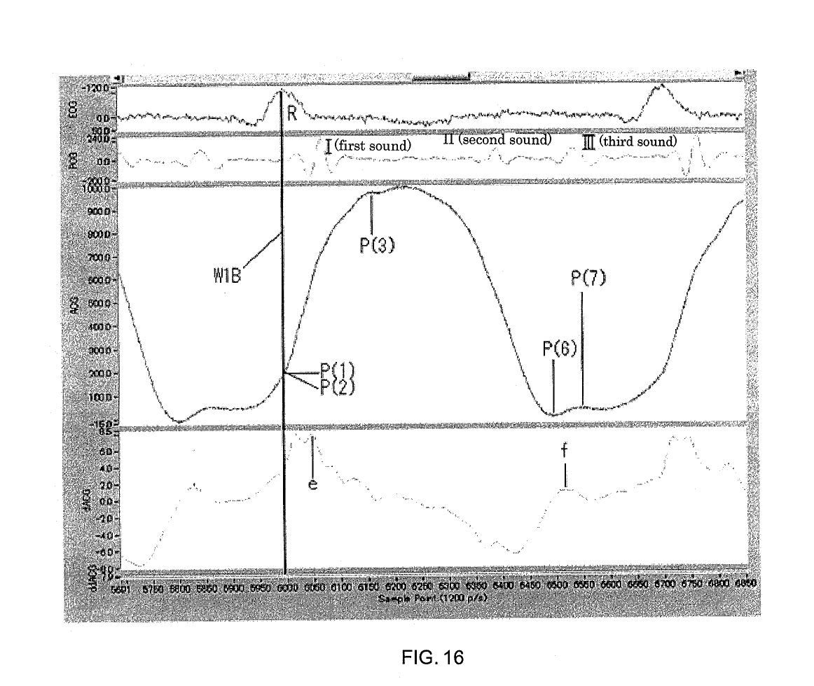

P(2) of apexcardiographic waveform is detected first. When an perpendicular line is drawn down from R in the electrocardiogram, a negative extreme (point where the first-order differential waveform changes from a negative value to a positive value) falling within a range of 20 ms before and after the point of intersection of the perpendicular line and the apexcardiographic waveform (a total of 40 ms) is defined as P(2). If there is no negative extreme within the range of 40 ms, the point of intersection of the perpendicular line and the apexcardiographic waveform is then defined as P(2). A positive extreme falling within a range of less than 160 ms immediately before P(2) (point where the first-order differential waveform changes from a positive value to a negative value) is determined as P(1). As illustrated in the example of FIG. 16, if wave A ascends continuously up to P(2) and there is no positive extreme (the first-order differential waveform of the apexcardiogram up to P(2) is positive), P(2) is defined as P(1).

Note that if it is required to find P(2) more securely, it is preferable to use “within the range of 30 ms after and before” instead of “within the range of 20 ms after and before.”

A positive extreme (point where the first-order differential waveform changes from a positive value to a negative value) falling between P(2) and the point less than 50 to 150 ms before P(2) is defined as P(3). If there is no P(3) within between P(2) and the point less than 50 to 150 ms before P(2), but there is a positive extreme between the point 150 ms after P(2) and the point before P(5), the positive extreme is defined as P(4). According to the full examination based on this inventor's measurement, in the case of human beings with normal left ventricle systole, distance from P(2) to P(3) is usually less than 125 ms. If the distance from P(2) to P(3) is less than 125 to 150 ms, left ventricle systolic function deteriorates, or left ventricular hypertrophy may exist so that special caution is required.

The case where neither P(3) nor P(5) exists but there is P(4) suggests it is abnormal. That is, there is a high possibility that left ventricular systolic dysfunction or left ventricular hypertrophy exists.

Next, (6) is detected. However, the minimum value that is separated 200 ms or greater away from the 2A sound is not defined as P(6). If 2-P(6) time (time from the 2A sound to the P(6))<150 ms holds true, it should be determined to be a normal value. If 150 ms<2-P(6) time<200 ms holds true, it should be determined to be an abnormal value.

Next, the P(7) is detected. The positive extreme that is separated 100 ms or greater away from the P(6) is not defined as P(7).

Whether each of the characteristic points exists depends on the living body to be measured and his/her health condition.

In FIGS. 15, P(1), P(2), P(3), P(5), P(6), and P(7) are identified respectively. P(1) is less than 300 points. P(2) is less than 300 points. P(3) exists in a range of less than 125 ms from P(2). There is a descending wave starting at P(3). There is a re-ascending wave including peak P(5) at a point 40 ms before the 2A sound (28 ms before according to data). This P(5) is P′ (5). There is a descending wave starting at P(5). P(6) is within a range of less than 150 ms from the 2A sound. P(7) is within a range of less than 100 ms from P(6). P(7) has a lower value than P(2). P(7) shows an overshoot.

Overshoot refers to a phenomenon where once a risen wave has reached a vertex, it temporarily falls downward acutely, and then rises again.

According to full examination based on the measurement of the present invention, these findings might be true in the case where there is normal left ventricular diastolic function with distensibility of the left ventricle (i.e., compliance is high), and also in the case of increased load in the early left ventricular diastolic phase. In the differential waveform X4, the interval between X5 and X6 is 40% or greater of the interval between P(2) and P(6). As before, a is less than ¼ of e, and is data indicating that the left atrial contraction force in the end diastolic phase is not abnormally increasing. f is less than ½ of e, and is data indicating that the load in left ventricular initial diastolic phase is not increasing. Moreover, a being smaller than f is important information in case of a young subject and is a normal finding.

In the first-order differential waveform obtained by differentiating the apexcardiogram in terms of time, the value of point a being less than ¼ of point e value is defined as normal, the value of the same falling within a region from ¼ or greater to less than ½ is defined to require caution or defined as being borderline, and the value of the same being ½ or greater is defined as abnormal. Moreover, the value of point f being less than ½ of the value of point e is defined as normal, the range from ½ or greater to less than ⅔ is defined as borderline or defined to require caution, and the region of ⅔ or greater is defined as abnormal.

In FIG. 15, the differential waveform X4 sharply drops from point e (maximum differential point) to point X5 (differential value point that is approximately zero) almost linearly, goes through a negative portion, changes to an approximately level state, and then goes back to a positive portion, reaching point X6 (differential value point that is approximately zero). From point X6, it drops until point L4 (minimum differential point) almost linearly, and then rises up almost linearly, going through L5 (differential value point that is approximately zero) to reach point f. Reference numeral L1 is a straight line segment connecting between point e and point X5, L2 is a straight line segment connecting between X5 and X6, and L3 is a straight line segment connecting between X6 and point X4.

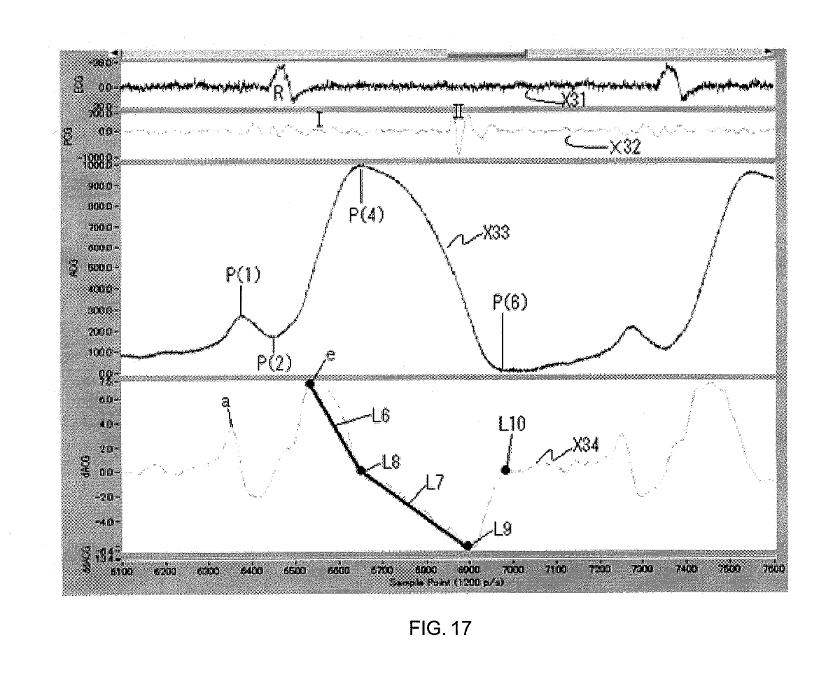

On the other hand, FIG. 17 shows an example in which the differential waveform of an apexcardiogram of the living body to be measured having a symptom different from the one shown in FIG. 15 is similarly examined as shown in FIG. 15. In FIG. 17, reference numeral X31 denotes an electrocardiogram, X32 denotes a phonocardiogram, X33 denotes an apexcardiogram, X34 denotes a differential waveform resulting from differentiating the apexcardiogram X33 by time, L8 and L10 denote a zero point (point zero) of the differential waveform X34, L9 is a minimum differential point, L6 is a straight line segment connecting between point e and point L8, and L7 is a straight line segment connecting between L8 and L9.

The differential waveform X34 descends from point e to point L8 (i.e., a point where differential value is approximately zero) almost linearly, and as in the case of X5-X6 of FIG. 15, drops until L9 almost linearly without any transition to an approximately level state, and then rises up almost linearly, reaching point L10.

As described above, FIG. 15 shows data of the living body to be measured having a normal ventricular function and FIG. 17 shows data of the living body to be measured who has a left ventricular systolic dysfunction. By comparing FIGS. 15 and 17, the differential waveform precipitously drops from point e to reach zero and then changes to an approximately level state in FIG. 15. In other words, L2 exists. By studying the differential waveform X4 of FIG. 15 in this manner, it is a characteristic point for supporting that P(3) and P(5) exist in the apexcardiogram, and suggests that the left ventricular systolic function is generally normal. On the other hand, the fact that the differential waveform X34 of FIG. 17 does not have a level interval from point e to point L9 supports that the waveform in systolic phase in the apexcardiogram is monopole in which only P(4) exists, and P(3) and P(5) do not exist, without having a line segment corresponding to the line segment L2 of FIG. 15. This is a characteristic finding that suggests left ventricular systolic dysfunction.

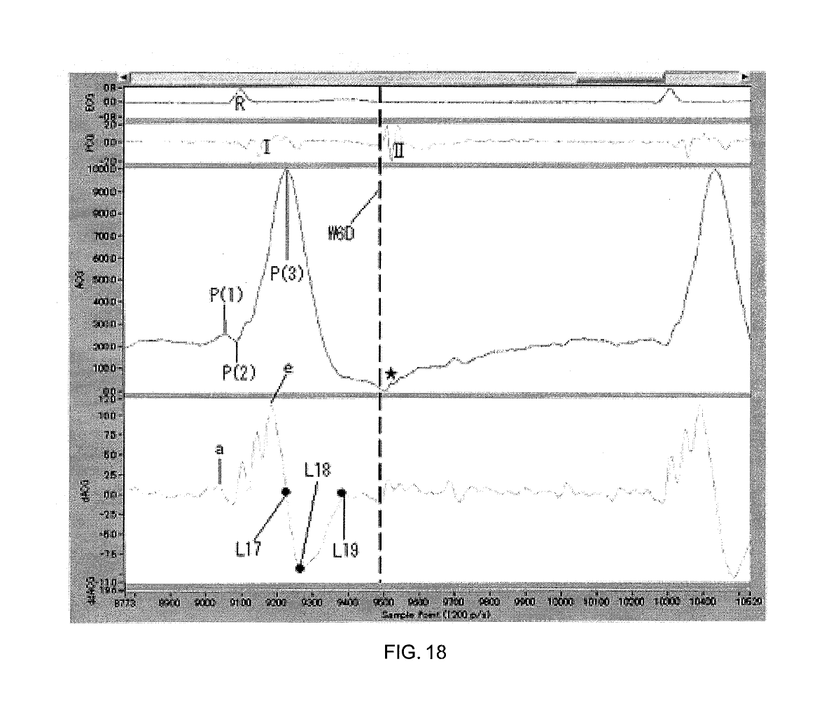

In FIG. 18, an example of a basic waveform of a different type than that of FIG. 17, having a single peak for a living body to be measured.

FIG. 18 shows an example of an apexcardiogram obtained by measuring for approximately 10 seconds using the biometric information measuring device according to the present invention; wherein a reference numeral W6D denotes a line indicating the position of the 2A sound. The horizontal axis and vertical axis are the same as in the case of FIG. 3. FIG. 18 shows measurement data of normal left ventricle systolic function of a male aged 30 years or below. The apexcardiogram includes a monopole systolic phase waveform.

By comparing an example in FIG. 18 and an example in FIG. 17 with respect to an apexcardiogram, in case of an apexcardiogram shown in FIG. 18, the minimum point L18 of the differential values of the first-order differential waveform in the apexcardiogram exists further on the left side of the midpoint between the two points L17 and L19 having a differential value of zero, namely exists in front of the time axis. In the case of the apexcardiogram of FIG. 17, there is a minimum point L9 among the differential values in the first-order differential waveform, which is located further right from the halfway point between two points L8 and L10 having a differential value of zero, or on later in time. There is a characteristic point P(3) in the example of FIG. 18, but not in the example of FIG. 17. There is no characteristic point P(4) in the example of FIG. 18, but there is in the example of FIG. 17.

In FIG. 18, there is a spike (indicated by an asterisk in the drawing), which corresponds to the second sound.

FIG. 18 shows a monopole systolic phase waveform, and features a characteristic point P(3) and a local minimum point among differential values in the first-order differential wave form located on the left hand side, which suggests that left ventricular systolic function is normal. On the other hand, FIG. 17 shows data of the living body having left ventricular systolic dysfunction; wherein there is a local minimum point among differential values located on the right hand side of a first-order differential waveform X34 for an apexcardiogram X33, which suggests left ventricular systolic dysfunction.

Next, attention is directed toward the horizontal axis (time axis) of the first-order differential waveform in FIG. 15.

The interval from P(5) to P(6), namely interval approximately corresponding to the isovolmic systolic phase corresponds to the interval between X6 and L5 (X6-L5 interval) in the first-order differential waveform. In the diastolic phases, we believe that the better the distensibility of the left ventricle, the earlier the relaxation will occur. Namely, we believe that the relaxation peak will appear in the early phase. Relaxation will start at P(5) in the apexcardiogram. We believe that the fact that the point L4 indicating maximum rate of change of the first-order differential waveform, which indicates the rate of change of that relaxation, is located in the front half portion of the X6-L5 interval means that aforementioned normal relaxation occurs in the early phase. As described above, the diastolic behavior in the isovolmic systolic phase may be characteristically and easily evaluated.

As clarified by the following description, the biometric information measuring device and the biometric information measurement system according to the present invention conduct apexcardiogram etc. at bedside for not only a healthy person and a person with light conditions of disease who can be taken into a “soundproof room”, but also a patient with a serious disease who is not expected at all conventionally to be taken into a measurement environment, such as a “soundproof room”. Moreover, they make a diagnosis of the health condition of the living body to be measured, which highly reliably agrees with the symptom medically diagnosed apart from the present invention, with the high degree of coincidence.

The apexcardiogram provided from the biometric information measuring device according to the present invention is useful for diagnosis of the left heart system, especially left ventricular function, and allows full examination of the left ventricular function by evaluating in time phase and height each of the characteristic points (P(1), P(2), P(3), P(5), P(6), and P(7), and point a, point e, and point f) of the measured example. Of them, especially, P(5), P′ (5), P″(5), P′″(5), and P″(5) play an important role. The inventor of the present invention has found through many measurements using the biometric information measuring device according to the present invention that: the case where “P(5)−2” time, which is the interval between the P(5) and the 2A sound, is less than 40 ms should be categorized into a normal group; the case where it is less than 40 to 50 ms should be categorized into high possibility of a normal group; the case where it is less than 50 to 70 ms should be categorized into both possibilities of a normal and an abnormal group; and the case where it is 70 ms or greater should be categorized into an abnormal group.

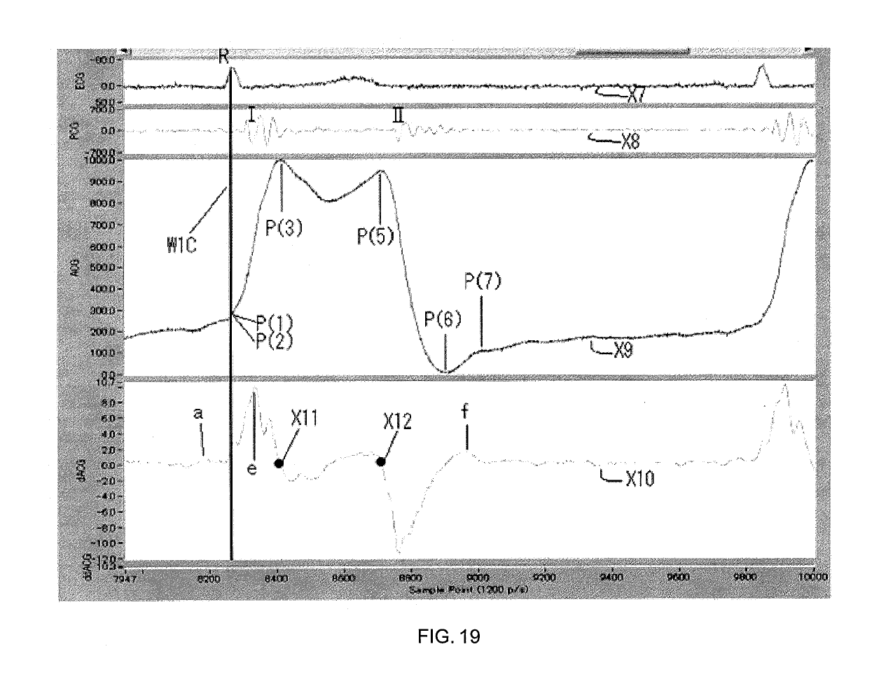

FIG. 19 illustrates an example of a male in his 50's having normal left ventricular function; wherein reference numeral W1C denotes a line indicating a QRS peak point. P(2), P(3), P(5), P(6), and P(7) are identified. Wave A having a slightly ascending convex waveform is recognized. Since it ascends up to P(2) but does not have a positive extreme, P(1) agrees with P(2). Distance from P(2) to P(3) is less than 125 ms so that it is determined as normal.

In a differential waveform X10, distance between X11 and X12 is 47% of distance between P(2) and P(6). This is an index indicating that the left ventricular function is maintained. P(7) looks like not having a clear overshoot. This suggests that distensibility of the left ventricle is lower than the example of FIG. 15. This is decrease in distensibility of the left ventricle due to aging so that it should be considered to be physiological normal finding. a is less than ¼ of e, which is data indicating that the left atrial contraction in the end diastolic phase is not strengthening abnormally. f is less than ½ of e, which is data indicating that the load in the early left ventricular diastolic phase is not increasing. Moreover, it is normal finding that a is smaller than f.

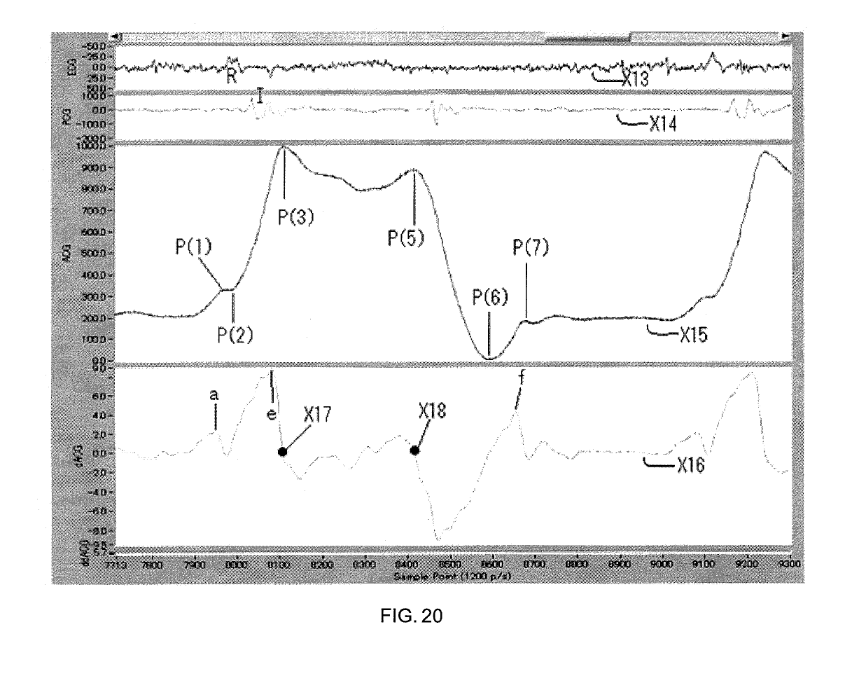

FIG. 20 shows an example of a living body to be examined: a 20-year old female having normal left ventricular function. P(1), P(2), P(3), P(5), P(6), and P(7), have been identified respectively. P(1) is 320 points. P(2) is 320 points. These are confirmed findings from an example of a young subject with normal left ventricular systolic function and very good left ventricular diastolic function. Since wave F is precipitous due to very good left ventricular diastolic function, difference between P(6) and P(7) is large. Moreover, since P(7) has a high value, P(1) and P(2) also have relatively high values. When point P(2) is a high value and difference in height between points P(1) and P(2) is small, analysis etc. is progressed by proceeding to a separately defined, predetermined procedure. P(3) exists less than 125 ms from P(2). A descending wave is observed starting at P(3). A re-ascending wave including P′(5) as the vertex P(5) 16 ms before the 2A sound is confirmed. A descending wave is observed starting at P(5). P(6) is confirmed at less than 150 ms from the 2A sound. P(7) is confirmed at less than 100 ms from P(6). P(7) is a lower value than P(2). P(7) is slightly overshoot. Measurement by the biometric information measuring device according to the present invention shows such findings often observed with young subjects having normal left ventricular functions, as in FIG. 15. This shows good elasticity of the left ventricle. In the differential waveform X16, the interval between the two points X17 and X18 that have a differential value zero for P(3) and P(5) is 52% of the P(2)-P(6) interval, and is an indicator that the left ventricular function is being maintained since this is 40% or greater of the P(2)-P(6) interval. a is less than ¼ of e, and is data indicating that the left atrial contraction in the end diastolic phase is not abnormally increasing. f is less than ½ of e, and is data indicating that the load in the early left ventricular diastolic phase is not increasing. Moreover, a being smaller than f is a normal finding.

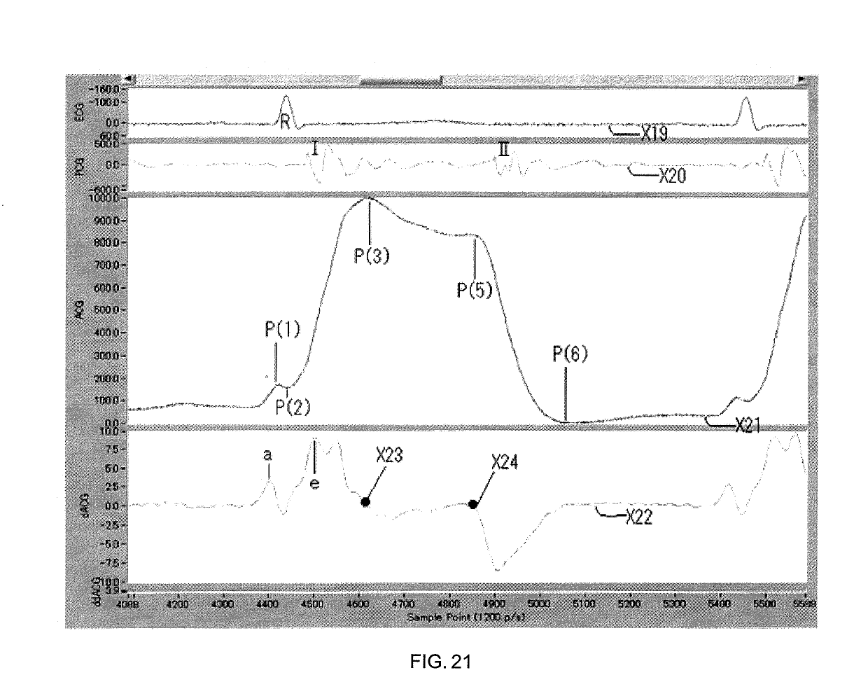

FIG. 21 is an apexcardiogram for a female in her 70's without previous heart disorder. P(1), P(2), P(3), P(5) and P(6) are identifiable, however, there is no P(7). This indicates that the left ventricular diastolic function is deteriorating. This is a finding that the left ventricular diastolic function is deteriorating. This finding is considered to be a physiological symptom due to aging. Moreover, distance from P(2) to P(3) is less than 125 to 150 ms, which indicates that there is possibility of left ventricular systolic dysfunction or left ventricular hypertrophy, and therefore it requires caution. In a differential waveform X22, distance between X23 and X24 is 40% of the distance between P(2) and P(6), which is an index indicating that the left ventricular function is maintained. Furthermore, since the value of point a falls within a range between a point of ¼ or greater than the value of the point e and a point of less than ½ of the value of the point e, there is possibility of left ventricular diastolic dysfunction in the end phase and therefore health condition is determined to require special caution and receive a medical specialist's diagnosis.

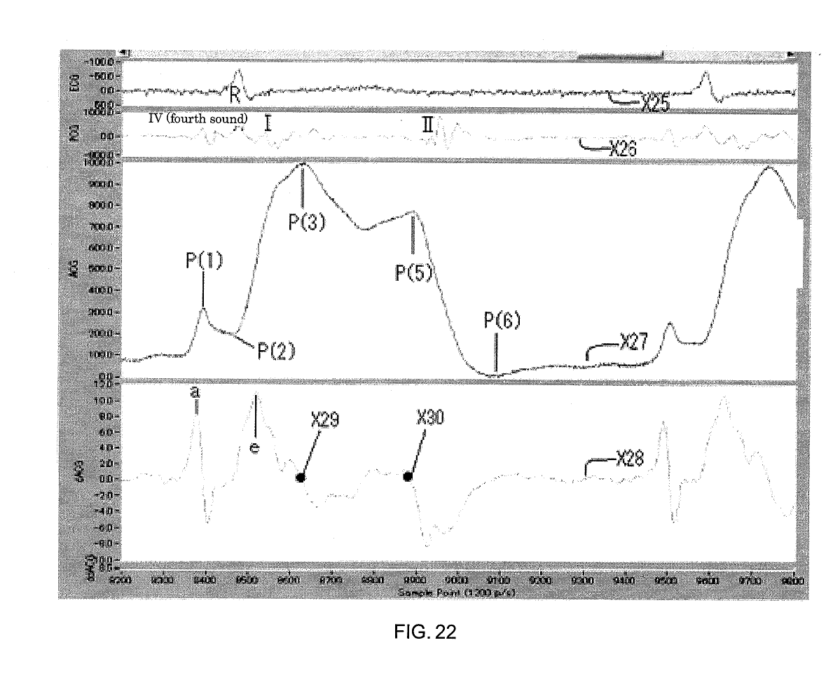

FIG. 22 illustrates an example of an apexcardiogram obtained by measuring a male in his 70's as a living body to be measured using the biometric information measuring device according to the present invention. Results from examining the health information of the data of FIG. 22, according to the present invention, are as follows. Since there are P(3) and P′ (5), possibility that left ventricular systolic function is normal is high. However, since P(2)-P(3) time is 125 ms or greater and less than 150 ms, there is possibility of left ventricular systolic dysfunction or left ventricular hypertrophy. P(7) is 50 points or less and is not identified, thereby suggesting mild early diastolic left ventricular dysfunction. Since P(1) is 300 points or greater, end diastolic left ventricular dysfunction is suggested. Moreover, since point a >point e/2 holds true, there is a chance of increased left atrial contraction. The health condition is determined to require special caution, recommending a medical specialist's diagnosis. Diagnosis for this living body to be measured by the medical specialist is hypertension in NYHA (New York Heart Association classification) functional classification class I, and therefore that judgment is appropriate.

Next, an example of the examined living body to be measured that experienced heart failure is given.

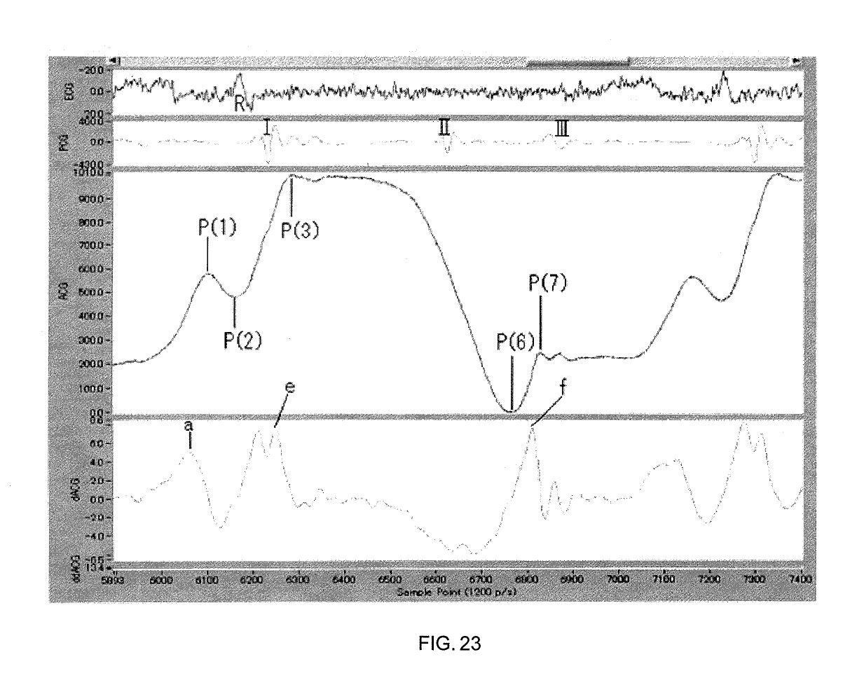

FIG. 23 is an example of an apexcardiogram obtained for a living body or a male in his forties using the biometric information measuring device according to the present invention. Results from examining the health information of the data of FIG. 23, according to the present invention, are as follows. The subject is not young, a monopole graph including P(3) but not P(5) is given, and of the width of the graph at 700 points when the height of P(3) is normalized to 1000 points, the section after P(3) in time phase (which is referred to as P3 rear width hereafter) is 250 ms, thereby suggesting either left ventricular systolic dysfunction or left ventricular hypertrophy. P(7) is 200 points or greater, f is greater than 2e/3, thereby suggesting early severe left ventricular diastolic dysfunction. Moreover, P(1) is 500 points or greater and P(2) is approximately 500 points, both of which exceed 300 points, thereby suggesting left ventricular end diastolic dysfunction. The physical condition is critical, and diagnosis by a specialist is recommended. This diagnosis by a specialist for the living body to be measured is dilated cardiomyopathy in New York Heart Association (NYHA) functional classification class 4, insisting on bed rest and oxygen inhalation, and thereby the aforementioned diagnosis is appropriate.

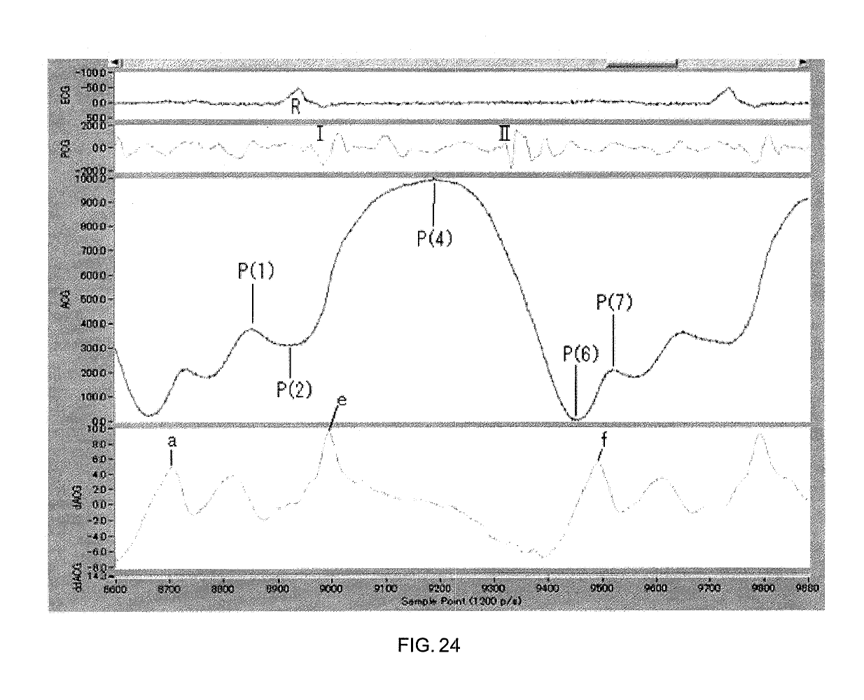

FIG. 24 is an example of an apexcardiogram obtained for a living body to be measured or a male in his fifties using the biometric information measuring device according to the present invention. Results from examining the health information of the data of FIG. 24, according to the present invention, are as follows. P(4) exists but there is no P(3) nor P(5), thereby suggesting either left ventricular systolic dysfunction or left ventricular hypertrophy. P(7) is 200 points or greater, the subject is not young, wave F is overshot, f is greater than 2e/3, thereby suggesting early severe left ventricular diastolic dysfunction. P(1) and P(2) are 300 points or greater, thereby suggesting left ventricular end diastolic dysfunction. a is e/4 or greater and less than e/2, indicating a chance of increased left atrial contraction. The physical condition is critical, and diagnosis by a specialist is recommended. This diagnosis by a specialist for the living body to be measured is ischemic cardiomyopathy in NYHA functional classification class 4, insisting on bed rest and oxygen inhalation, and thereby the aforementioned diagnosis is appropriate.

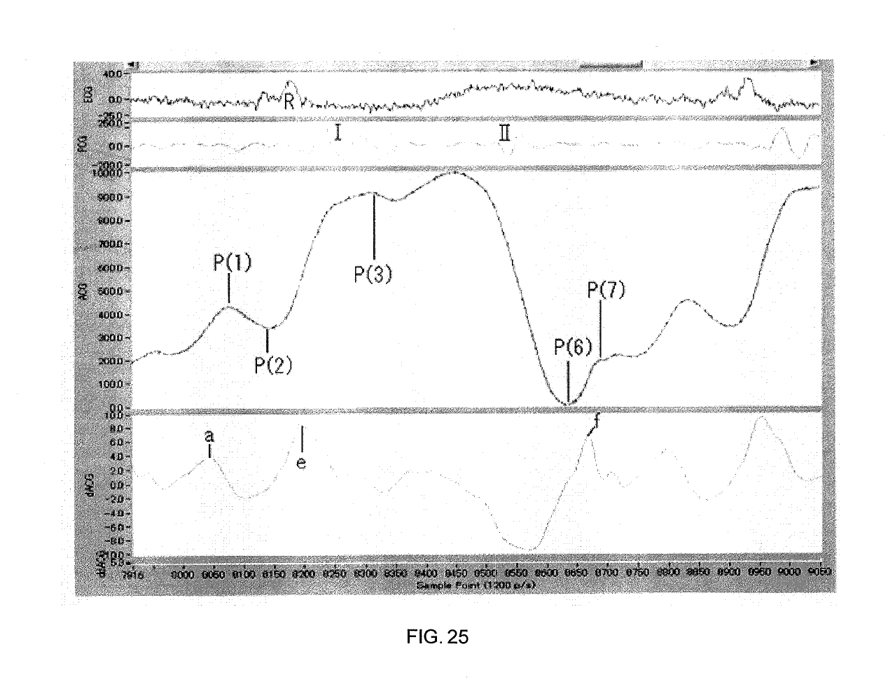

FIG. 25 is an example of an apexcardiogram obtained for a living body to be measured or a male in his sixties using the biometric information measuring device according to the present invention. Results from examining the health information of the data of FIG. 25, according to the present invention, are as follows. P(3) is visually unclear, however, the measuring device has determined that P(3) exists and that P(5) does not exist, thereby suggesting the chance of either decreased left ventricular systolic function or left ventricular hypertrophy. P(7) is 190 points, which means 50 points or greater and less than 200 points, however, since f is greater than 2e/3, the possibility of early diastolic left ventricular dysfunction should be considered. P(1) and P(2) are greater than 300 points, thereby suggesting left ventricular end diastolic dysfunction. Moreover, a is e/4 or greater and less than e/2, indicating a chance of increased left atrial contraction. The physical condition requires examination, and diagnosis by a specialist is recommended. This diagnosis by a specialist for the living body is dilated cardiomyopathy in NYHA functional classification class 3, exhibiting shortness of breath from several meters of level ground walking, and thereby the aforementioned diagnosis is appropriate.

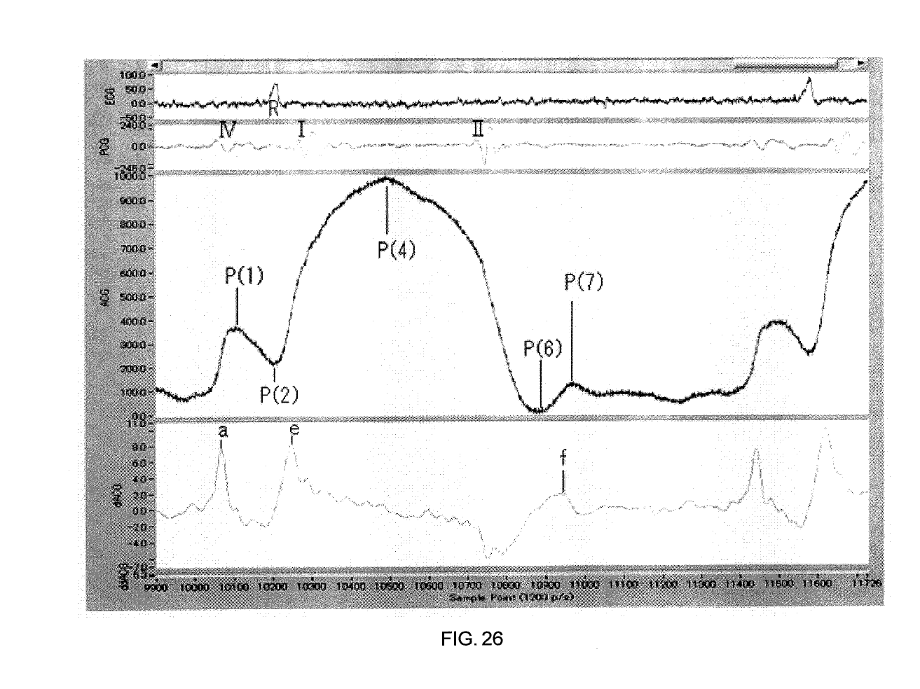

FIG. 26 is an example of an apexcardiogram obtained for a living body to be measured or a female in her seventies using the biometric information measuring device according to the present invention. Results from examining the health information of the data of FIG. 26, according to the present invention, are as follows. P(4) exists but there is no P(3) nor P(5), thereby suggesting either decreased left ventricular systolic function or left ventricular hypertrophy. P(7) is 110 points, which is a normal value by the standards of the present invention, thereby concluding that early diastolic left ventricular dysfunction is unlikely. While P(2) is 300 points or less, P(1) is greater than 300 points, thereby suggesting left ventricular end diastolic dysfunction. Point a is greater than e/2, thereby suggesting increased left atrial contraction. Point f is approximately e/4, and thereby the probability of early diastolic left ventricular dysfunction is low. The physical condition requires caution, and diagnosis by a specialist is recommended. This diagnosis by a specialist for the living body is hypertonia, arteriosclerosis obliterans, and old apical myocardial infarction in NYHA functional classification class 2, and thereby the aforementioned diagnosis is appropriate.

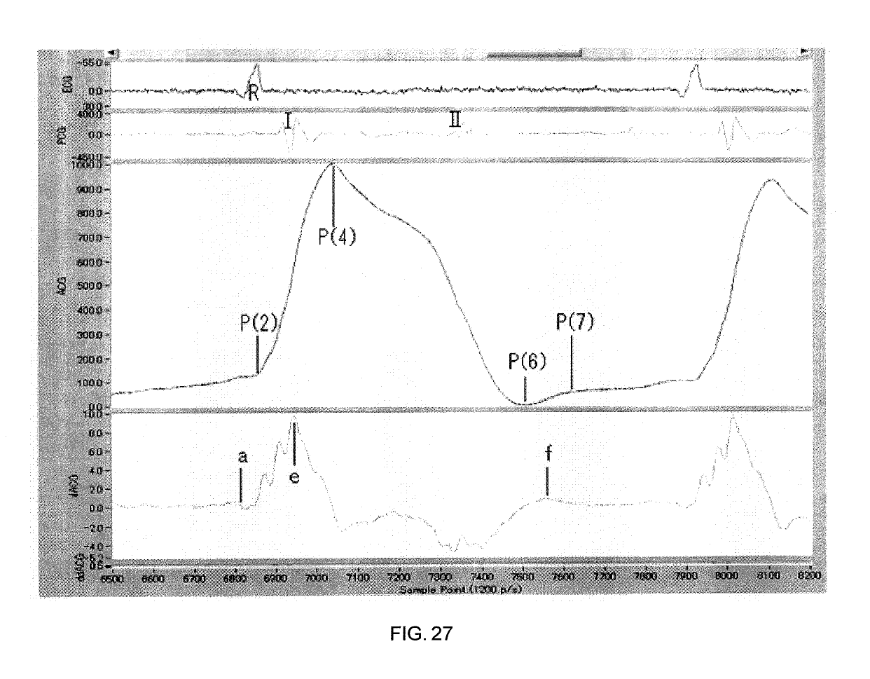

FIG. 27 is an example of an apexcardiogram obtained for a living body to be measured or a female in her sixties using the biometric information measuring device according to the present invention. Results from examining the health information of the data of FIG. 27, according to the present invention, are as follows. P(4) exists but there is no P(3) nor P(5), thereby suggesting either left ventricular systolic dysfunction or left ventricular hypertrophy. P(7) is barely distinguishable with 50 points, point f is low, and thereby the probability of early diastolic left ventricular dysfunction is low. P(1) and P(2) are both 150 points or less, and thereby the probability of left ventricular end diastolic dysfunction is low. a is small, and thereby the probability of increased left atrial contraction is low. The physical condition requires examination, and diagnosis by a specialist is recommended. This diagnosis by a specialist for the living body is dilated cardiomyopathy in NYHA functional classification class 2, allowing less than moderate physical activity, and thereby the aforementioned diagnosis is appropriate.

Next, two examples of old myocardial infarction are given.

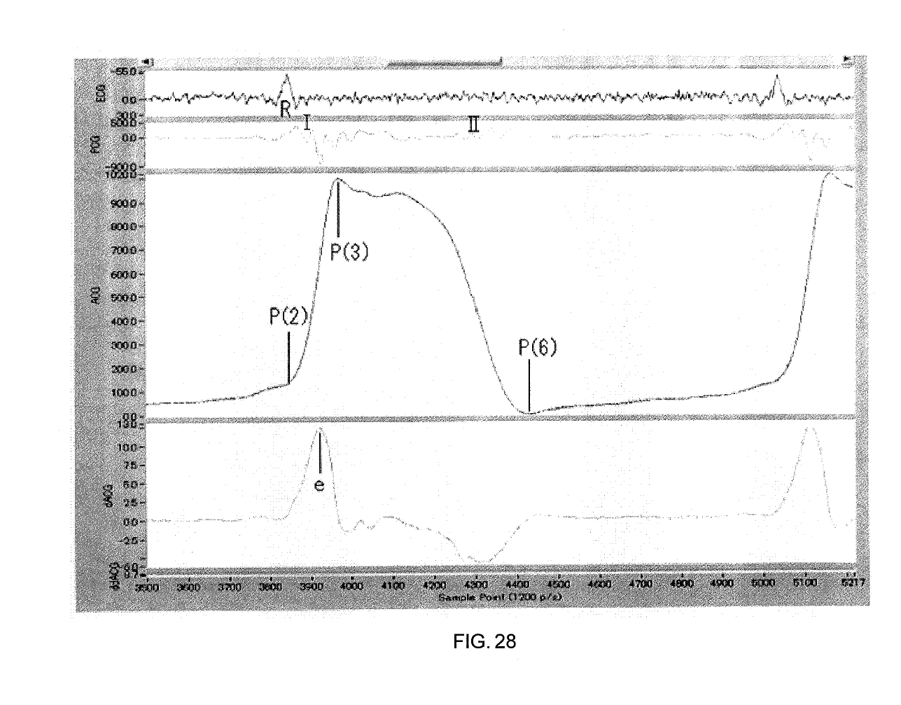

FIG. 28 is an example of an apexcardiogram obtained for a living body to be measured or a male in his late sixties using the biometric information measuring device according to the present invention. The subject is not young, a monopole graph including P(3) but not P(5) is given, and the width of the P3 rear width is 250 ms, which is substantially wider than 100 ms, thereby suggesting either left ventricular systolic dysfunction or left ventricular hypertrophy. P(6) is barely distinguishable, and neither P(7) nor point f exists, thereby suggesting mild early diastolic left ventricular dysfunction. Point e is observed but point a is not, and thereby the probability of left ventricular end diastolic dysfunction is low. The physical condition requires examination, and diagnosis by a specialist is recommended. This diagnosis by a specialist for the living body is old anteroseptal myocardial infarction, experiencing compensated heart failure in NYHA functional classification class 3, and thereby the aforementioned judgment is appropriate.

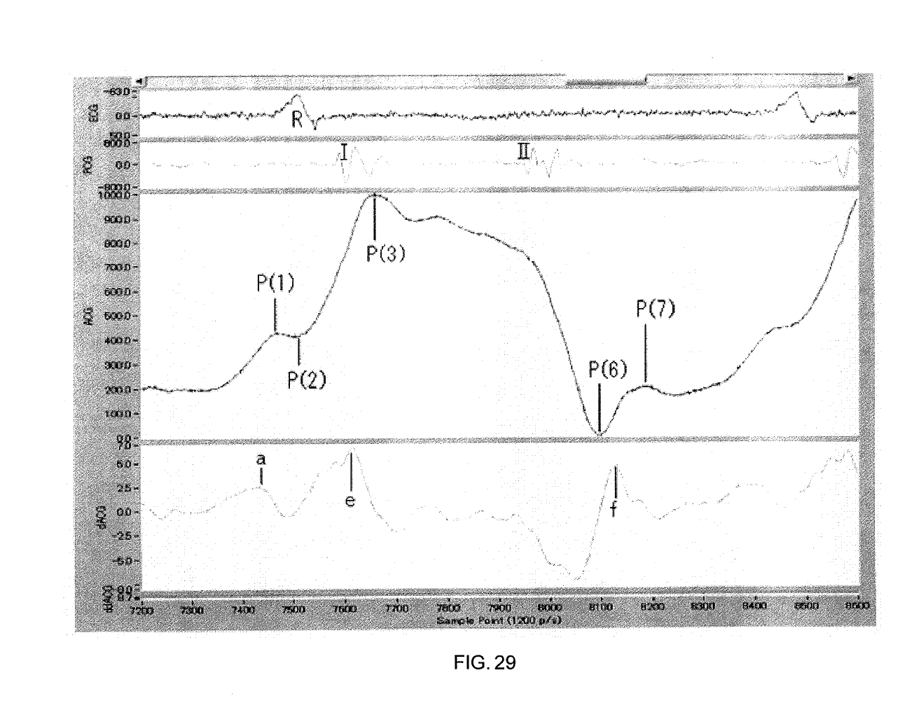

FIG. 29 is an example of an apexcardiogram obtained for a living body to be measured or a male in his late sixties using the biometric information measuring device according to the present invention. The subject is not young, a monopole graph including P(3) but not P(5) is given, and the width of the P3 rear width is 270 ms, which is substantially greater than 100 ms, thereby suggesting either left ventricular systolic dysfunction or left ventricular hypertrophy. f is greater than 2e/3, thereby suggesting left ventricular systolic dysfunction with or without left ventricular hypertrophy. P(6) exists, P(7) is 200 points, the subject is not young, and there is overshoot, thereby suggesting early diastolic left ventricular dysfunction. The fact that point f is greater than 2e/3 supports this. P(1) and P(2) are 400 points or greater, thereby suggesting left ventricular end diastolic dysfunction. e/2>a>e/4 holds true, thereby suggesting a chance of increased left atrial contraction. The physical condition is critical, and diagnosis by a specialist is recommended. This diagnosis by a specialist for the examined living body is old anteroseptal myocardial infarction, experiencing decompensated heart failure, which is different from the example of FIG. 28, in NYHA functional classification class 4, and thereby the aforementioned judgment is appropriate.

Next, an example of hypertrophic nonobstructive cardiomyopathy is given.

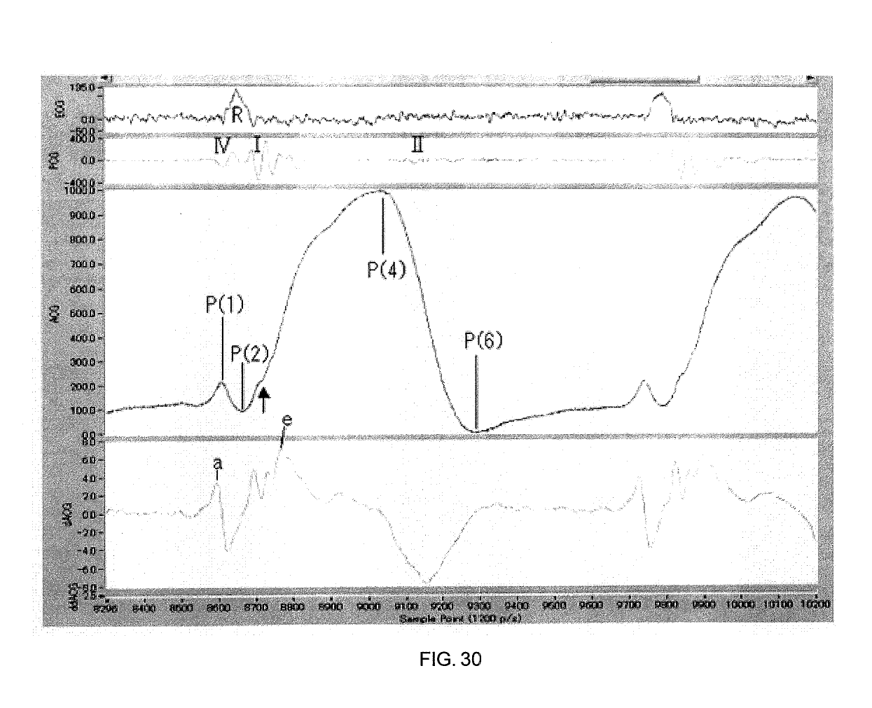

FIG. 30 is an example of an apexcardiogram recorded for a living body to be measured or a male in his early seventies using the biometric information measuring device according to the present invention.

P(4) exists but there is no P(3) nor P(5), thereby suggesting either left ventricular systolic dysfunction or left ventricular hypertrophy. The fact that P(7) does not exist suggests mild early left ventricular diastolic dysfunction. P(1) and P(2) are 300 points, but point a is greater than e/2, thereby suggesting left ventricular end diastolic dysfunction due to increased end-phase left atrial contraction. The physical condition requires examination, and diagnosis by a specialist is recommended. This diagnosis by a specialist for the living body is hypertrophic nonobstructive cardiomyopathy in NYHA functional classification class 2, exhibiting shortness of breath during physical activity. Thereby, the aforementioned diagnosis is appropriate.

Next, four examples of valvular heart diseases are given.

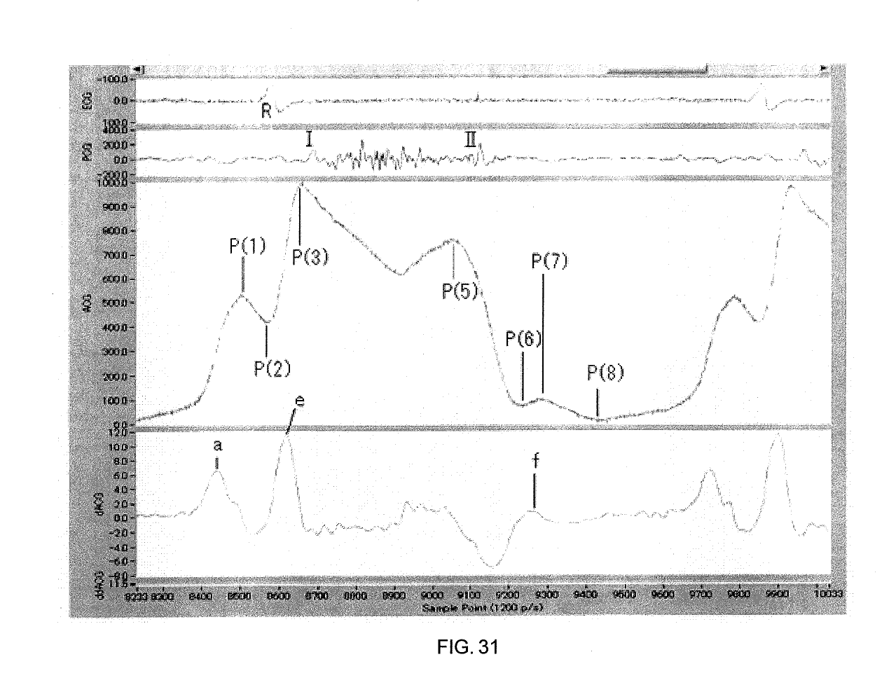

FIG. 31 shows an example of an apexcardiogram recorded for a 72-year old female. P(1) and P(2) are both high values, and point a of the first-order differential curve shows half or greater of point e, thereby suggesting end diastolic left ventricular dysfunction due to increased left atrial contraction. The fact that P(7) is a low value suggests early diastolic left ventricular dysfunction. The physical condition is critical, and diagnosis by a specialist is recommended. This diagnosis by a specialist for the living body is severe aortic stenosis. Thereby, the aforementioned diagnosis is appropriate.

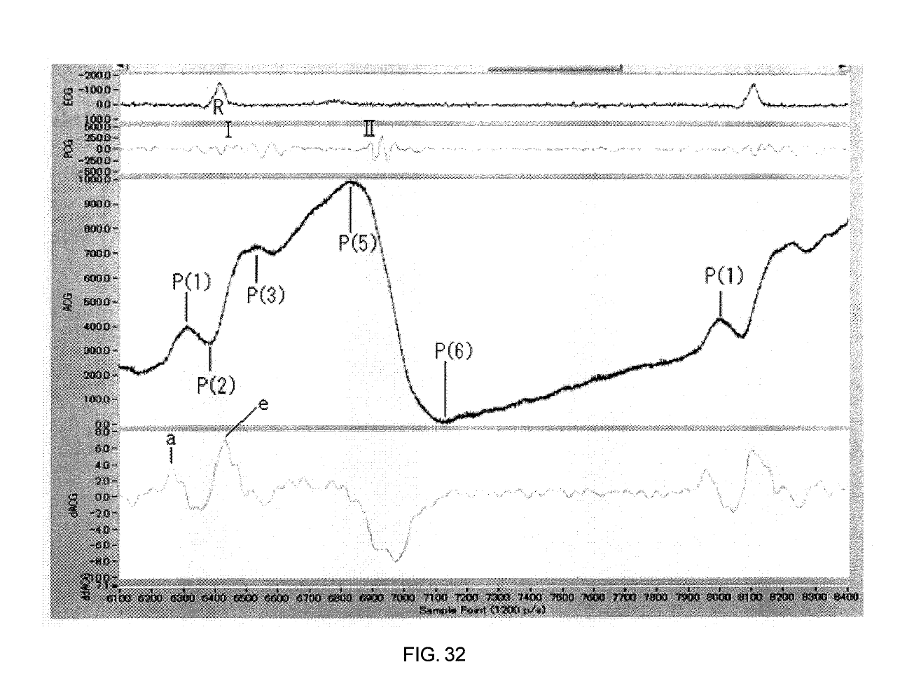

FIG. 32 shows an example of an apexcardiogram recorded for a male in his early seventies. P(1) and P(2) are both high values, and point a of the first-order differential curve shows half or greater of point e, thereby suggesting end diastolic left ventricular dysfunction due to increased left atrial contraction. The fact that P(7) is unclear suggests early diastolic left ventricular dysfunction. A characteristic of this patient in the diastolic phases is expressing a waveform that continually rises from P(6) until P(1) of the next unit waveform. This suggests sustained left ventricular pressure through all of the diastolic phases. The physical condition requires examination, and diagnosis by a specialist is recommended. This diagnosis by a specialist for the living body is severe aortic regurgitation. Thereby, the aforementioned diagnosis is appropriate.

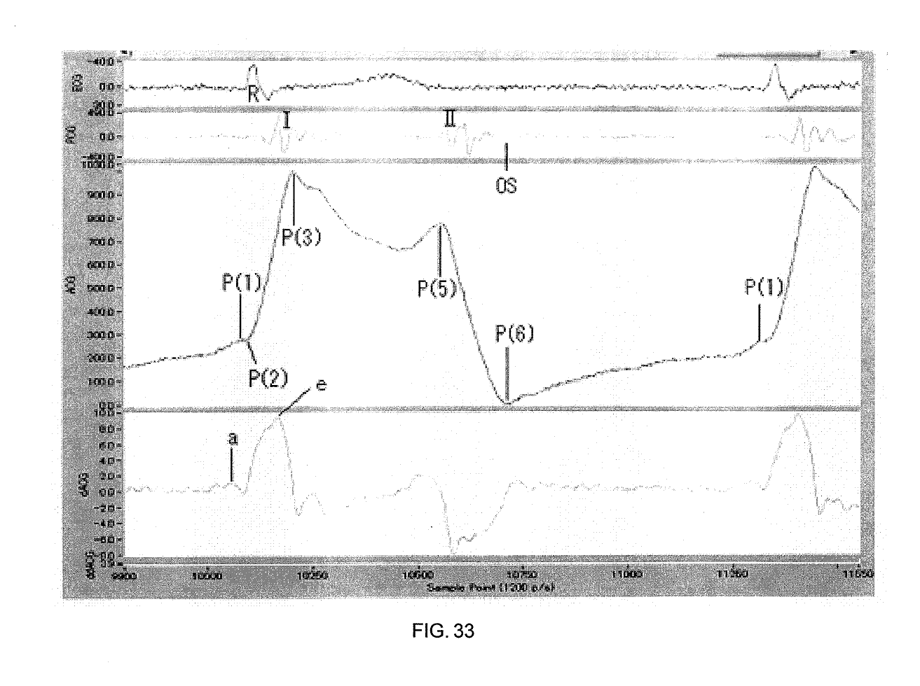

FIG. 33 shows an example of an apexcardiogram recorded for a 60-year old male. P(3) appeares in the early phase, having a pointed shape, which could be considered due to the vibration of the increased first sound. The value of point a is very low. P(1) and P(2) are both less than 300 points, and thereby the probability of left ventricular end diastolic load is low. Wave F can not be recognized, and a wave continuously rising from wave A is formed. A mitral opening snap, although small, is confirmed in the phonocardiogram shown by adding OS. The physical condition requires examination, and diagnosis by a specialist is recommended. This diagnosis by a specialist for the living body is moderate mitral stenosis. Thereby, the aforementioned diagnosis is appropriate.

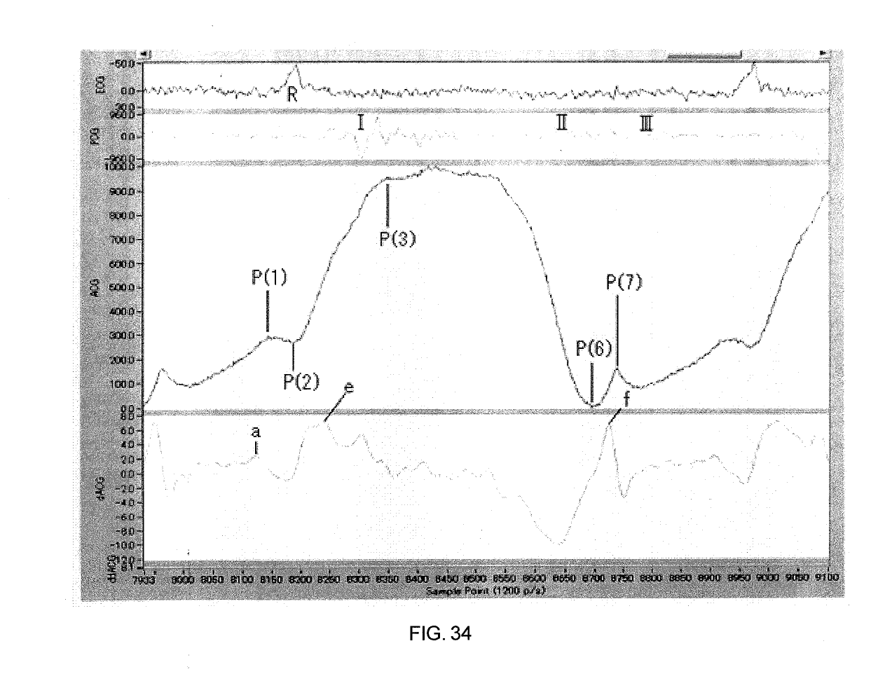

FIG. 34 shows an example for a 61-year old female having severe mitral regurgitation and has suffered from heart failure. P(1) and P(2) are both under 300 points. Point a is between a quarter and a half of point e, thereby suggesting left ventricular end diastolic load due to left atrial contraction. Wave E takes on roundness, and P(3) is unclear. P(5) does not exist. The fact that P(5) can not be confirmed indicates either left ventricular systolic dysfunction, or high probability of left ventricular hypertrophy while maintaining left ventricular systolic function. P(7) is sharp and there is overshoot. Point f is remarkably high, exceeding two-thirds the height of point e.

The physical condition requires examination, and diagnosis by a specialist is recommended. This diagnosis by a specialist for the living body is severe mitral regurgitation and has suffered from heart failure. Thereby, the aforementioned diagnosis is appropriate.

The data analysis algorithm according to the examples of the present invention is further described. First, the existence of P(5) in an apexcardiogram is checked. P(5) is located less than 70 ms just before the 2A sound of the phonocardiogram in the apexcardiogram and is a positive extreme (the differential value is zero when it shifts from positive to negative). P(5) means the end of a systolic phase, and simultaneously means the beginning of a diastolic phase. P(5) is an indicator for early left ventricular diastolic dysfunction as well as an indicator for left ventricular systolic dysfunction. When P(5) exists, there is a low probability of severe left ventricular dysfunctions (systolic dysfunction and diastolic dysfunction).

If P(5) does not exist, it suggests left ventricular diastolic dysfunction. Moreover, in addition to left ventricular diastolic dysfunction, left ventricular systolic dysfunction is also suggested. Furthermore, a case where there is left ventricular hypertrophy and left ventricular contraction is normal is suspected.

Next, P(6) is described.

Generally, P(6) is a minimum point on an apexcardiogram observed in the initial diastolic phase. The interval from the 2A sound to P(6) is an indicator representing left ventricular diastolic function. This shows distensibility of the left ventricular muscle. If the interval from the 2A sound to P(6) is long, it means that the distensibility of the left ventricular muscle has decreased. The interval from the 2A sound to P(6) is judged as normal if it is less than 150 ms, and is judged as abnormal if it is 150 ms or greater and less than 200 ms. The initial negative extreme at a distance of 200 ms or greater from the 2A sound is not regarded as P(6).

Next, wave F is described.

Wave F is a relatively precipitously rising wave starting at P(6) in the apexcardiogram, and is formed by rapid blood flow from the left atrium to the left ventricle in the initial diastolic phase. P(7) is the vertex of wave F. The end of wave F almost coincides with the third sound in time phase. Point f is a positive peak value of wave F of the first-order differential waveform in the apexcardiogram.

In an apexcardiogram that slowly gradually rises and does not show any bending point, it is judged that no wave F is confirmed. This case is determined that neither P(7) nor point f is included. When wave F is not confirmed, early diastolic left ventricular dysfunction is diagnosed. The case where P(7) is equal to or greater than P(2) in height is diagnosed as abnormal. The case where P(7) is less than P(2) is diagnosed as normal. The case where P(7) is less than 50 points is diagnosed as abnormal. This finding suggests left ventricular diastolic dysfunction meaning a state where the left ventricle has difficulty in expanding in the early diastolic phase. The case where P(7) is 50 points or greater and less than 200 points is diagnosed as normal. The case where P(7) is 200 points or greater is diagnosed as abnormal. When the living body to be measured is a human under 30 years old, as described above, there are cases where less than 230 points is diagnosed as normal and 230 points or greater is diagnosed as abnormal.

The case where P(7) is 200 points or greater suggests left ventricular diastolic dysfunction meaning increased early diastolic load. This finding that P(7) is 200 points or greater may be confirmed in an example of normal left ventricular function, in specific, good distensibility of the left ventricular muscle. Moreover, it indicates a pathological condition of early diastolic left ventricular dysfunction.

Next, point f is described.

The case where the height of point f is less than half the height of point e is diagnosed as normal. The case where the height of point f is half or greater than the height of point e and less than two-thirds the height of point e is diagnosed as a marginal zone and requiring caution. The case where the height of point f is at least two-thirds the height of point e is diagnosed as abnormal. The case where the height of point f is at least half the height of point e suggests early diastolic left ventricular dysfunction.

Wave F, P(7), and point f are all indicators of early diastolic left ventricular dysfunction.

The more severe the heart failure, the more noticeable (meaning high values) wave F, P(7), and point f are noticeable (meaning high values) and the earlier they appear in an earlier time phase. However, as an exception, there are cases indicating a young subject with normal left ventricular function and extremely good distensibility of the left ventricular muscle.

Next, P(1) is described.

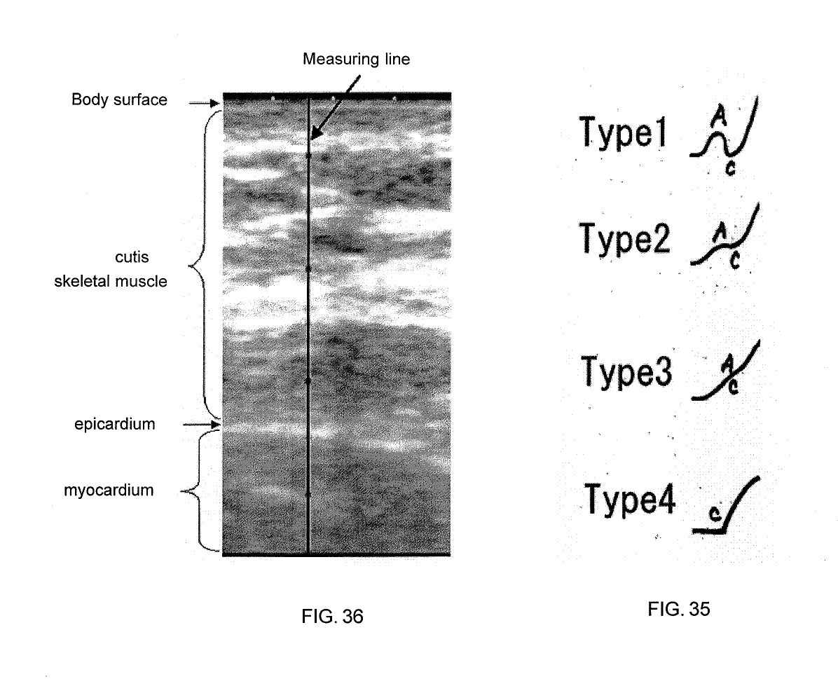

Classifications of wave A are given in FIG. 35. The types of wave A are classified into the following four groups. Type 1 is the case where wave A is clear, P(1), which is the location of the positive extreme, is clear, and P(2) is also clear. Type 2 is a rising waveform from the rise of wave A until P(2). P(1) and P(2) are approximately the same height, but the portion corresponding to P(2) in the first-order differential waveform includes a negative extreme (differential value shifts over from negative to positive). Type 3 is a waveform where wave A continuously rises until P(2), and the portion corresponding to P(2) in the first-order differential waveform does not include a negative extreme (differential value shifts over from negative to positive). P(1) and P(2) are defined identical in this case. Type 4 is the case where there is hardly any positive wave observed before P(2), and it is judged that no wave A is confirmed.

When there is no wave A, namely when there is hardly any positive wave observed before P(2), it is judged that wave A does not exist, and neither P(1) nor point a exist. There is a normal case where there is no load in the left ventricular end diastolic phase, and a pathological case where left atrial systolic function has been lost or decreased.

When wave A is confirmed, P(1) exists and point a exists as well. As type 3, P(1) and P(2) may be identical (when wave A continuously rises, reaching P(2)).

The case where the height of P(1) is 300 points or greater is diagnosed as abnormal. The case where the height of P(1) is 300 points or greater suggests left ventricular end diastolic dysfunction.

The case where the height of P(1) is less than 300 points is diagnosed as normal.

Next, point a is described.

The case where the height of point a is half or greater than the height of point e is clearly diagnosed as abnormal. The fact that point a is high suggests a rise in left ventricular pressure due to increased left atrial contraction in the end diastolic phase. The case where the height of point a is at least one-quarter and less than half the height of point e is diagnosed as a marginal zone and caution required along the borderline. The case where the height of point a is less than one-quarter of the height of point e is diagnosed as normal. When point a is lower than point f, either a case of normal left ventricular function, in specific, extremely good left ventricular diastolic function, or the opposite case of severe heart failure (decompensated failure) is possible.

P(1), point a, and P(2) are all indicators of left ventricular end diastolic dysfunction. Since P(1), point a and P(2) are associated, study of the mutual relationship of these three items are thought to allow classification of pathological conditions as given below. In the case of type 1 and type 2 shown in FIG. 35, when P(1) is high, P(2) is high, and point a is high, it suggests increased left atrial contraction and rise in left ventricular end diastolic pressure. When P(1) is high, P(2) is high, and point a is low, it suggests left ventricular end diastolic pressure without increased left atrial contraction. When P(1) is high, P(2) is normal, and point a is high, it suggests increased left atrial contraction. When P(1) is high, P(2) is normal, and point a is low, it suggests end diastolic left ventricular dysfunction is mild. When P(1) is low, P(2) is normal, and point a is low, it suggests no load in the left ventricular end diastolic phase. When P(1) is low, P(2) is normal, and point a is high, it suggests increased left atrial contraction.

As type 3, in the case where P(1) and P(2) are identical, when P(2) is high and point a is low, it suggests that end diastolic left ventricular dysfunction is mild. When P(2) is high and point a is high, it suggests increased left atrial contraction and rise in left ventricular end diastolic pressure. When P(2) is low and point a is low, it suggests there is no end diastolic left ventricular dysfunction. When P(2) is low and point a is high, it suggests increased left atrial contraction.

Next, P(2) is described.

The case where P(2) is 300 points or greater is diagnosed as abnormal. There is a chance of rise in left ventricular end diastolic pressure.

Next, P(3) and P(4) are described.

If P(2) to P(3) is less than 125 ms, a low probability of left ventricular systolic dysfunction is suggested. The case where P(2) to P(3) is between 125 ms and 150 ms requires caution and there is a possibility of left ventricular systolic dysfunction, or otherwise there is a chance of left ventricular hypertrophy even when there is no left ventricular systolic dysfunction. In the case where P(3) does not exist but P(4) exists, it will be diagnosed as left ventricular systolic dysfunction, or otherwise there is a high probability of left ventricular systolic dysfunction even when there is no left ventricular systolic dysfunction, and it will thus be diagnosed as abnormal.

With the biometric information measuring device according to the present invention, once basic data of an apexcardiogram is measured, recorded, and loaded into the device, the health condition of the living body to be measured may be diagnosed, recorded, and displayed as required using items related to the respective characteristic points described above.

As described before, the first-order differential waveform of the apexcardiogram, namely the first-order differential waveform of the beat waveform over time may be utilized for extracting characteristics of heartbeats of the living body to be measured, and second-order differential waveform of the apexcardiogram may be utilized for extraction and the like of characteristic points of the first-order differential waveform. For diagnosis of a mechanocardiogram, in addition to use of waveforms of the apexcardiogram as data in sync with an electrocardiogram or a phonocardiogram, it is of extreme importance to use the first-order differential and second-order differential thereof, as well as information of amplitude of beats, amplitude distribution, intensity, and intensity distribution for accurate diagnosis.

Understanding the apexcardiogram, amplitude of apex, and distribution of the amplitude are necessary for understanding cardiac movement. Moreover, it is also needed to understand the intensity of drive and the intensity distribution such as whether the heart is moving vigorously, forcefully, etc. As well-known by doctors who conduct palpation, the heart moves in a complicated manner, and being able to detect the intensity of heartbeats that can be felt through palpation as energy in the movement, strength in the movement etc. of the heart and the intensity distribution using the pressure sensor according to the present invention is important information for carrying out precise diagnosis.

The embodiments may have various applications as needed, and results that could not be anticipated conventionally may be demonstrated.

For example, after apexcardiogram is conducted, results may be explained to the patient promptly, and the apexcardiogram may be explained while showing it to the patient. Moreover, previous data of a living body to be measured or useful statistical data for diagnosis of the living body to be measured may be loaded as reference data into the device according to the present invention so as to manage health effectively by ranking the health condition of the living body to be measured, grasping change in the health condition, and explaining it to the examined living body to be measured.

Various sensors such as a wave sensor may be incorporated in a pressure sensor. Use of such a structure not only allows high precision, simplification, down-size, price-reduction etc. of the measuring device, it also allows drastically alleviating mental load due to measuring by loading many sensors onto the living body to be measured.

By making the pressure sensor have a three-dimensional structure, pressure, heart beats, etc. may be detected, and the S/N ratio of measured data may be increased.

It is possible to structure the device according to the present invention by incorporating a transmitter or a transmitter/receiver to a heartbeat sensor having various configurations as described above and loading it on the living body to be measured, or loading the heartbeat sensor and a small lightweight transmitter or transmitter/receiver on the living body to be measured, and transmitting or transmitting and receiving data measured by a sensor to an external device carried by the living body to be measured or an external device located at a distance from the living body to be measured, so that it is useful in health management by the user of the device, and can carry out diagnosis and health management of the living body to be measured by a medical professional.

In measurement of apexcardiogram, in the case where the device is equipped with sensors for electrocardiography and phonocardiography, and even in the case where data for electrocardiogram and phonocardiogram is obtained through measurement independently from the device according to the present invention and then input to the device according to the present invention, It is important to synchronize the apexcardiogram at least with either the electrocardiogram or the phonocardiogram. By doing so, highly reliable assessment may be conducted.

An example of conducting apexcardiogram using a pressure sensor, which utilizes ultrasound, is explained, next.

FIG. 36 is a tomogram obtained through measurement using ultrasound, according to an embodiment of the present invention, wherein ultrasonic probes are placed against the chest wall of a living body to be measured, so as to take the picture of a part of the heart. In this figure, locations of a body surface, a cutis and a skeletal muscle, an epicardium, and a myocardium in each part of the layers are indicated.

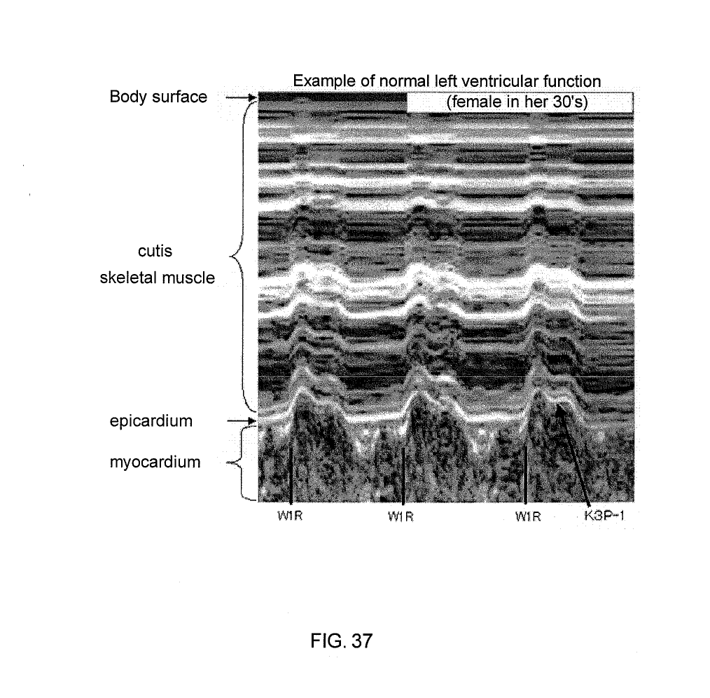

FIG. 37 is a diagram provided by measurement of time-phased change in a position, described as a measuring line in FIG. 36, along time axis orthogonal to the layer length; wherein reference numeral W1R is a line indicating the QRS peak location. Reference numeral K3P-1 is data of measured movement of the epicardium portion over time on the position indicated by the measuring line of FIG. 36. Such data is not discussed academically; however, as described later, it shows movement corresponding to the apexcardiogram, and since the apexcardiogram shows this measured movement that is conveyed to the body surface as pressure, the diagram represented by the reference numeral K3P-1 according to the present invention is included in the apexcardiogram, as described above.

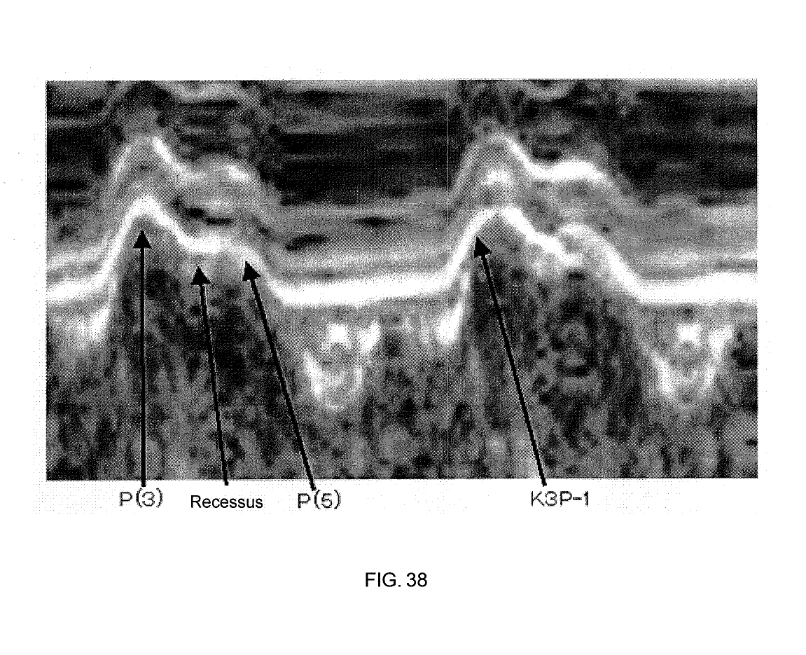

FIG. 38 is a diagram showing enlarged substantial parts of the apexcardiogram K3P-1 of FIG. 37. This is an apexcardiogram including a recessus between the characteristic points P(3) and P(5).

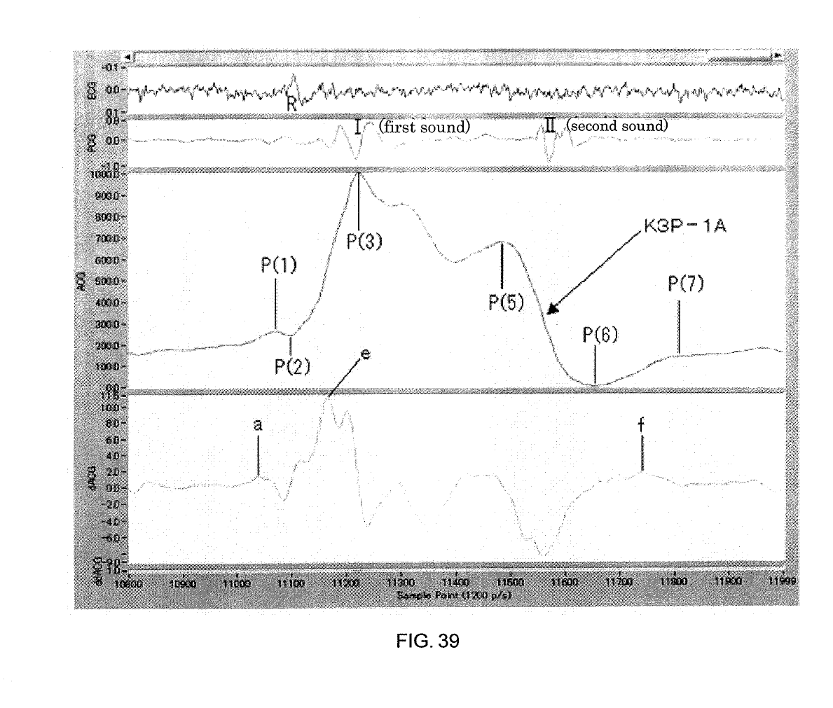

FIG. 39 is an apexcardiogram for the same living body measured using a pressure sensor, which is not for transmitting ultrasound waves, at almost the same time on the same day as FIG. 38, where reference numeral K3P-1A denotes the apexcardiogram. The pressure sensor is similar to pressure sensor that measured the apexcardiograms illustrated in FIGS. 15 and 32, etc.

FIG. 38 has low resolution since it is measured not using a dedicated sensor such as an ultrasonic sensor, but using a transportable first prototype, which is manufactured utilizing the probes for a commercially available ultrasonograph for another purpose. However, the characteristic points P(3) and P(5) observed in an apexcardiogram of FIG. 39 obtained using the pressure sensor that measured the same living body at almost the same time on the same day, are clearly identified, where P(3) is evidently higher than P(5). It is confirmed that through further data processing, tendencies of the characteristic points P(2), P(1), P(6), and P(7) may be understood. This example is recorded for a thirty-year old female with normal left ventricular function.

The biometric information measuring device according to the present invention used for FIGS. 36 to 39 includes an analysis means for characteristic points etc. in accordance with the algorithm of the present invention for the apexcardiogram, and a function as an ultrasound diagnostic device. Therefore, this allows addition of the diagnosis based on the measured data from the ultrasound diagnostic device to diagnosis based on the apexcardiogram, and also allows, based thereon, extraction of health information of the assessed living body. As a result, extraction of health information of the assessed living body is possible with higher reliability.

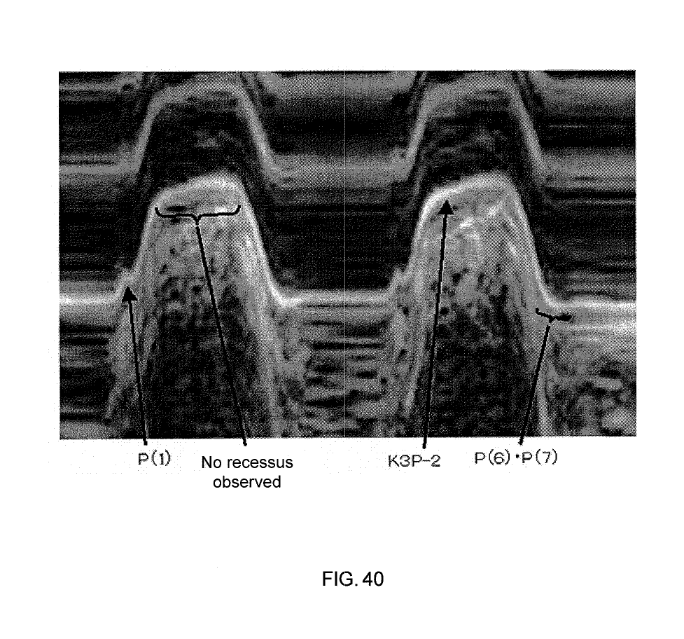

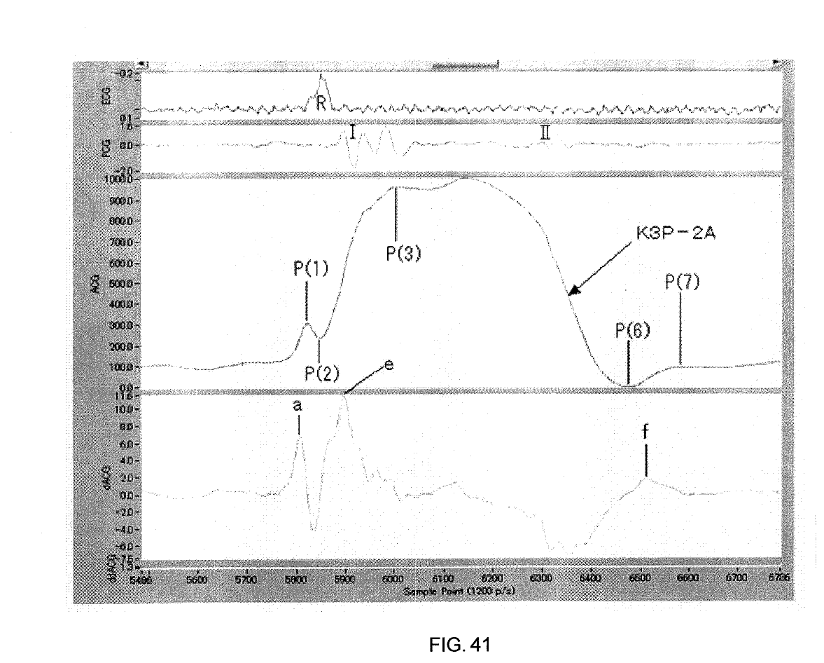

FIG. 40 is an apexcardiogram recorded by placing an ultrasonic sensor, which is a heartbeat sensor or a pressure sensor, on the chest wall of a living body using the biometric information measuring device according to an embodiment of the present invention. FIG. 41 is an apexcardiogram recorded by placing a pressure sensor on the chest wall of a living body using the biometric information measuring device according to an embodiment of the present invention, where K3P-2 and K3P-2A denote the apexcardiograms. The characteristic points P(1) and P(2) are clear, wave A is clear, but P(7) is unclear. There is no recessus observed in the left ventricular systolic phase. This living body is in late seventies, and the data thereof included symptoms of old anteroseptal myocardial infarction diagnosed by a specialist.

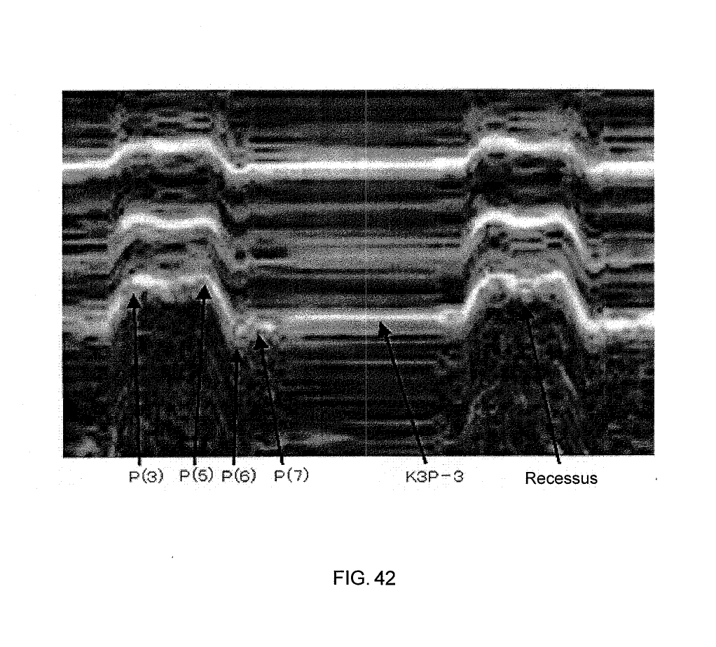

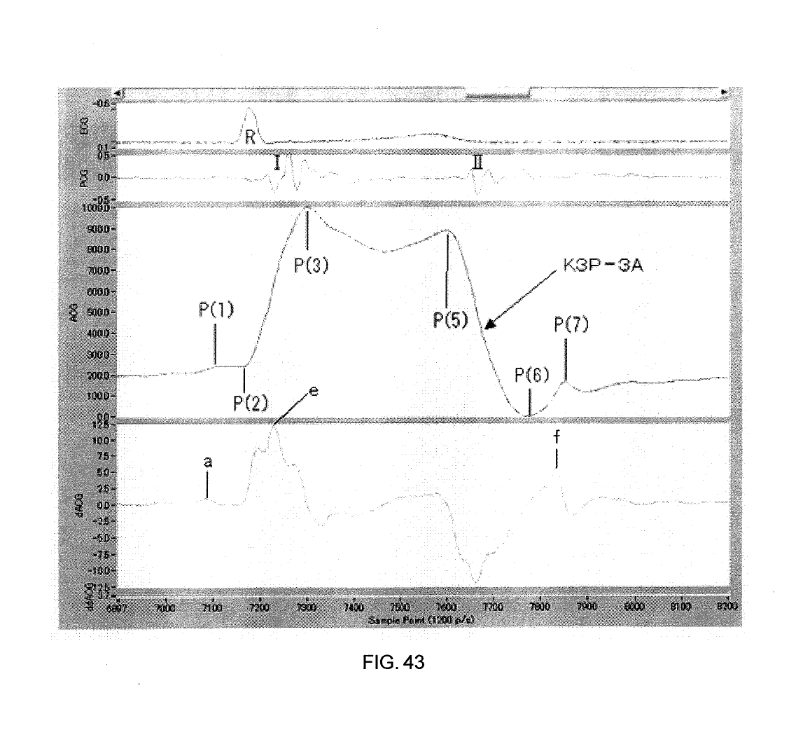

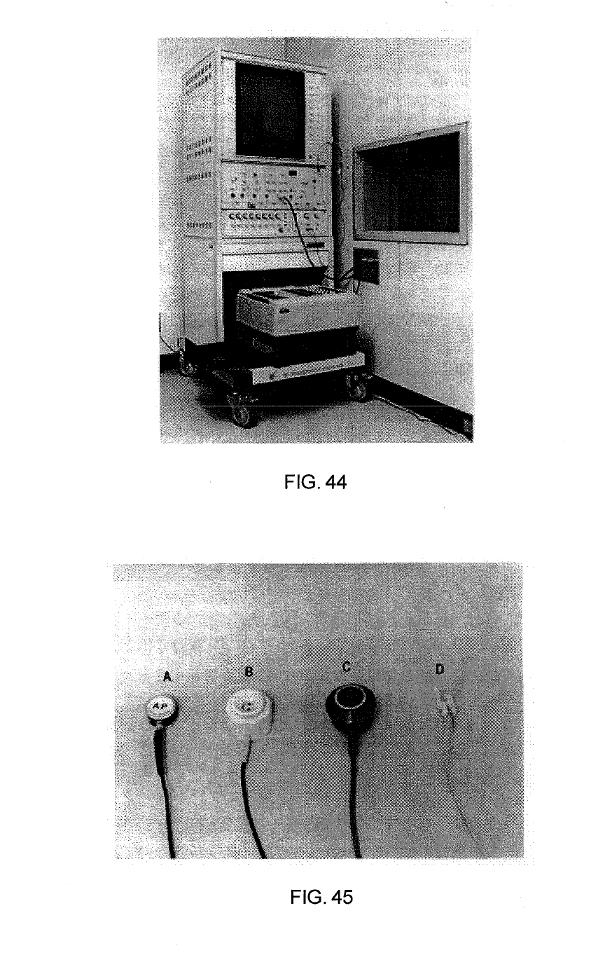

FIG. 42 is an apexcardiogram recorded by placing an ultrasonic sensor on the chest wall of a living body using the biometric information measuring device according to an embodiment of the present invention. FIG. 43 is an apexcardiogram recorded by placing a pressure sensor, which is not for transmitting ultrasound waves, on the chest wall of a living body using the biometric information measuring device according to an embodiment of the present invention; wherein K3P-3 and K3P-3A denote apexcardiograms. In FIG. 42, characteristic points P(1) and P(2) are unclear, P(3) and P(5) are clear with recessus therebetween, and P(6) and P(7) are clear, corresponding to the apexcardiogram of FIG. 43. This example is recorded for a male in his 10's with normal left ventricular function.

Furthermore, according to the present invention, an optical sensor may be used as the wave sensor. The composition of the sensor unit is explained further.

As a preferable example of the optical sensor, an optical fiber for transmission, a light source, or multiple optical receiving units arranged in the periphery may be used. According to a preferable example of the present invention, the optical fiber for transmission is used, and/or a laser beam of 0.8 micrometer in wavelength is also used as a light source to propagate the optical fiber, and as multiple optical receiving units, multiple optical fibers for reception arranged surrounding around the optical fiber for transmission three deep so that the angles to respective normals on the surface of a living body to be measured differ from one another.

Linear polarized light or circular light may be used as the laser beam for transmission. Alternatively, pulsed light may be used as the laser beam for transmission. These contribute to improvement in the S/N ratio of the detected laser beam.

A preferable example of an optical sensor comprises at least a collimator arranged at the receiving light inputting end of the optical fiber for reception, there by improving the S/N ratio of the detected laser beam entering from the prescribed direction.

Alternatively, a preferable example of the optical sensor may be constituted by an optical fiber shared for transmission and reception etc.

Moreover, deployment of non-reciprocal elements, such as a Faraday cell, on the collimator may improve the S/N ratio of the detected laser beam.

Especially in the case of measurement on the chest wall, it is preferable to arrange them so as for the transmitting light enters the living body along multiple axes at mutually different angles of incidence.

As such, the biometric information measuring device of the present invention not only conducts apexcardiogram, but also examines a living body from many angles using ultrasound or light, thereby providing health information with high reliability.

As explained above, the present invention allows miniaturization of the measuring device and lowering of the cost, allows a medical practitioner to conduct apexcardiogram even in a hospital outpatient clinic or a patient's room, and allows feedback to patients on the spot, resulting in provision of extremely great advantageous effects as a medical treatment, enhancing the quality of medical examination skills, such as ocular inspection, palpation, and auscultation, and making it possible to give accurate diagnoses for the circulatory disease.

Furthermore, the present invention provides extremely great advantageous effects for lifelong education for medical practitioners and clinical medicine education for medical students and intern doctors.

Furthermore, the present invention allows a variety of applications of the medical technical knowledge, such as ordinary health care or remote health care.

The electronic components according to the present invention have been explained so far based on the embodiments according to present invention. However, the present invention is not narrowly limited to the above-mentioned embodiments. While the present invention is explained by showing data of human beings, for example, so as to exemplify the extremely great results when a living body to be measured is a human being, the present invention is not limited to the embodiments based on the technical ideas according to the present invention, and there are many variations based on using those technical ideas.

### INDUSTRIAL APPLICABILITY

The present invention provides remarkably improved medical treatment of the circulatory system, and enhances cooperating results with the other medical fields, thereby contributing to progress in medicine very well and greatly saving the health care cost, that is, providing economical advantageous effects. Therefore, the present invention may be widely used in the medical fields and the medical education fields as well as the health-care equipment fields.

### REFERENCE NUMERALS

P(1)˜P(8): Characteristic Points of Apexcardiogram
ACG,K3,K3A-K3G,K3J,K3P-1,K3P-1A,K3P-2,K3P-2A,K3P-3,K3P-3A,X3,X9,X15,X21,X27,X33: Apexca rdiogram
PCG,K2,K2G,X2,X8,X14,X20,X26,X32: Phonocardiogram
ECG,K1,K1C-G,K1J,X1,X7,X13,X19,X25,X31: Electrocardiogram
DACG,X4,X10,X16,X22,X28,X34: First-order Differential Waveform of the Apexcardiogram
K11-19,K22-27,K29-44,K49-54,K56-60: Unit Waveform
W1A,W1C,W1D,W1E,W1R: Lines indicating QRS Peak Positions
201a,260,301: Pressure Sensor
241a˜241c,242a˜242c,243a˜243c, 261:
244a˜244d: Sensor Unit for Detection of Contact Pressure
249a˜249d: Fixing Point Correction Means
300: Biometric Information Measuring Device
305a: Cardiac Sound Sensor
305b: Sensor for Electrocardiogram
320: Control/Measurement Data Processing Unit
330: Memory Unit
340: Display Unit

1. A biometric information measuring device for measuring and recording the heartbeat of a living body, which has a heartbeat sensor for measuring motion or pressure of at least a part of the living body at which the heartbeat is to be detected, based on change in the position of the part or change in the pressure, the heartbeat of a living body is measured by the heartbeat sensor in sync with an electrocardiogram and health information of a living body is extracted from a signal measured by the heartbeat sensor, wherein the signal measured by the heartbeat sensor or an amplified signal of the signal are referred to as a heartbeat sensor output signal collectively hereafter the apexcardiographic waveform as one beat of measurement data, which is in sync with the electrocardiographic waveform, is defined as a basic waveform, and the basic waveform is displayed with the horizontal and the vertical axis representing time and amplitude of the apexcardiographic waveform, respectively; wherein the direction from a characteristic point P(2) to a characteristic point P(3) is positive on an apexcardiogram of healthy persons,
if there is a minimum point C1 on the apexcardiographic waveform within the range of 30 ms (millisecond) after and before the point of the basic waveform, which is referred to as a QRS peak point hereafter and corresponds to each QRS positive vertex value (R) of I or II lead of standard 12 leads electrocardiogram, C1 is defined as the characteristic point P(2), if the C1 is not clear, the point corresponding to the QRS peak point on the apexcardiographic waveform is defined as the characteristic point P(2);
a positive vertex on the apexcardiographic waveform between the QRS peak point and the point 160 ms before the QRS peak point (same hereafter) is defined as a characteristic point P(1);
a positive vertex on the apexcardiographic waveform between the P(2) and the point 50 ms to 150 ms after the P(2) is defined as a characteristic point P(3);
a positive vertex on the apexcardiographic waveform nearest to the 2A sound, which is an aortic-atresia sound on the phonocardiogram, between the 2A sound and the point less than 70 ms before the 2A sound is defined as a characteristic point P(5);
regarding P(5), if the P(5) is a positive vertex nearest to the 2A sound on the phonocardiogram between the 2A sound and the point less than 40 ms before the 2A sound, that positive vertex is then defined as a characteristic point P′(5);
and if the P(5) is a positive vertex nearest to the 2A sound on the phonocardiogram between the 2A sound and the point less than 40 ms to 170 ms before the 2A sound, the positive vertex is defined as a characteristic point P″(5), wherein the P(5), the P′(5), or the P″(5) are referred to as P(5) respectively or collectively, except for the case where the characteristic-points P′(5) and P″(5) are needed to be distinguished;
a positive vertex on the apexcardiographic waveform between the point 150 ms after the P(2) and the point 70 ms before the 2A sound is defined as a characteristic point P(4);
a negative extreme on the apexcardiographic waveform between the 2A sound and the point 50 to 150 ms after the 2A sound is defined as a characteristic point P(6);
if, between the 2A sound and 100 to 240 ms after the 2A sound, the P(6) exists, the positive vertex located after the P(6) on the apexcardiographic waveform is defined as a characteristic point P(7);
the characteristic points P(1), P(2), P(3), P(4), P(5), P′ (5), P″(5), P(6), and P(7) are defined as a first characteristic-point group;
a waveform risen before the P(1), which is a positive vertex on the apexcardiogram, is defined as a wave A; a waveform risen from the P(2) and having a positive extreme on the apexcardiogram is defined as a wave E; and an ascending wave starting at the P(6) is defined as a wave F;
a positive peak position of the wave A of the first-order differential waveform on the apexcardiogram is defined as a point a; a positive peak position of the wave E is defined as a point e; a positive peak position of the wave F is defined as a point f; and heights of the a, the e, and the f points are defined as a, e, and f, respectively;
a first characteristic-point determinator is a determinator for determining whether there are at least two characteristic points in the first characteristic-point group;
a second characteristic-point determinator is a determinator for defining ordinate value of the minimum position of the basic waveform as zero and determining height of at least one of the P(1), the P(2), and the P(7) when normalized, so as for the maximum coordinate value of the basic waveform to be 1000 points,
a third characteristic-point determinator is a determinator for defining, regarding time of each of the characteristic points, P(2)-P(3) time (this is time from the P(2) to the P(3), same hereafter), ratio of P(3)-P(5) time to P(2)-P(6) time, P(6)-P(7) time, 2-P(6) time (this is time from the 2A sound to the P(6), same hereafter), and 2-P(7) time as characteristic factors, and determining the value of at least one of the factors;
a first waveform determinator is a determinator for comparing to a waveform determination pattern stored in the biometric information measuring device or an apexcardiographic waveform determination pattern input to the biometric information measuring device from the outside, and determining the type of the basic waveform;
a second waveform determinator is a determinator for determining the value of each of the a, the e, and the f;
a third waveform determinator is a determinator for determining whether there is a section of the first-order differential waveform on the apexcardiogram running almost horizontally near a point having the first-order differential value of zero between the point e and the lowest point immediately before the point f;
a fourth waveform determinator is a determinator for determining whether the lowest point immediately before the point f of the first-order differential waveform on the apexcardiogram falls within the first half of the section between the point having a first-order differential value of zero immediately before the lowest point and the point having a first-order differential value of zero immediately after the lowest point;
a fifth waveform determinator is a determinator for determining whether the apexcardiogram includes a monopole graph not having P(5) but P(3), wherein if there is the monopole graph, whether the section after P(3) in time phase which is referred to as P3 rear width hereafter, of the width of the graph at 700 points when the height of P(3) is normalized to 1000 points is less than 100 ms; and
a data processor for extracting health information of a living body from the heartbeat sensor output signal comprises the first to the third characteristic-point determinators, at least one of the five determinators of the first to the fifth waveform determinators, and a health condition determinator for the living body to be measured.

2. (canceled)

3. The biometric information measuring device according to claim 1, wherein the data processor comprises at least the first characteristic-point determinator, and at least either the characteristic-point determinator or the waveform determinator, and determines regarding the P(3), the P(4), and the P(5) using the characteristic-point determinator and the waveform determinator, determines regarding the P(6) and P(7), and then determines regarding the P(1) and P(2), thereby assessing the health condition of an living body to be measured.

4. (canceled)

5. The biometric information measuring device according to claim 1, wherein the third characteristic-point determinator uses at least one of criteria: whether either the P(2)-P(3) time is 50 ms or greater and less than 125 ms or falls between 125 ms and 150 ms, whether either the P(3)-P(5) time is not less than 45% of the P(2)-P(6) time, 40% or greater and less than 45%, or less than 40%, whether the P(6)-P(7) time is less than 100 ms, or 100 ms or greater and less than 150 ms, whether either the 2-P(6) time is less than 150 ms, or 150 ms or greater and less than 200 ms, and whether either the 2-P(7) time is less than 240 ms, or 240 ms or greater.

6. The biometric information measuring device according to claim 1, wherein information processing is carried out by defining that:
possibility of severe left ventricular dysfunctions (systolic dysfunction and diastolic dysfunction) is low if there are the P(3) and the P′(5) on the basic waveform, and if there is no P′(5), left ventricular diastolic dysfunction is suggested, and/or left ventricular systolic dysfunction is suggested other than left ventricular diastolic dysfunction, and/or there is left ventricular hypertrophy and therefore determination as normal left ventricular contraction is suspected.

7. The biometric information measuring device according to claim 1, wherein information processing is carried out by defining that left ventricular systolic function is normal if the P3 rear width is less than 100 ms.

8. The biometric information measuring device according to claim 1, wherein information processing is carried out by defining that health condition of the living body is normal if the 2-P(6) time is less than 150 ms, otherwise, the health condition of the living body is abnormal if the 2-P(6) time is 150 ms or greater and less than 200 ms.

9. The biometric information measuring device according to claim 1, wherein information processing is carried out by defining that: the time interval between P(6) and P(7) is defined as a P(6)-P(7) time if there is the P(6); the health condition of the living body is normal if the P(6)-P(7) time is less than 100 ms; and the health condition of the living body is abnormal if the P(6)-P(7) time is 100 ms or greater and less than 150 ms.

10. The biometric information measuring device according to claim 1, wherein the second biometric information measuring device uses at least one of the criteria: regarding the a, the health condition is normal if the a is less than e/4; the health condition requires caution if the a is e/4 or greater and less than e/2;
the health condition is abnormal if the a is e/2 or greater; and
regarding the f, the health condition is normal if the f is less than e/2; the health condition requires caution if the f is e/2 or greater and less than 2e/3; and the health condition is abnormal if the f is 2e/3 or greater.

11. The biometric information measuring device according to claim 1, wherein information processing is carried out by determining that: the health condition of the living body is normal if there is a section of the first-order differential waveform of the apexcardiogram running almost horizontally near the point having the first-order differential value of zero between the point e and the lowest point immediately before the point f, otherwise the health condition of the living body is abnormal if that section does not exist.

12. (canceled)

13. The biometric information measuring device according to claim 1, wherein if there is neither the P(3) nor the P(5), but there is the P(4), which is a positive extreme, at the point 150 ms or greater from the P(2), the health condition is determined to be abnormal, displaying to that effect.

14. The biometric information measuring device according to claim 1, further comprising a determinator for determining whether the characteristic point has a predetermined range selected in time phase and height, and each measured data falls in the predetermined range, regarding at least one of the characteristic points the P(1) to the P(7) on the apexcardiogram and/or the characteristic points a, e, and f on the first-order differential waveform.

15. The biometric information measuring device according to claim 1, further comprising: a modifier for modifying the criteria.

16. (canceled)

17. The biometric information measuring device according to claim 1, further comprising an input device for inputting and setting predetermined ranges in time phase and height using a graphic symbol through an input part, such as a tablet, which is used by an examiner, regarding at least one of the characteristic points the P(1) to the P(7) on the apexcardiogram and/or the characteristic points a, e, and f on the first-order differential waveform, and a determinator for determining whether each measured data falls in the predetermined ranges.

18. The biometric information measuring device according to claim 1, wherein the wave sensor is an ultrasonic sensor, which receives ultrasound, and the biometric information measuring device comprises an ultrasound transmitter, an ultrasound receiver, and an ultrasonic echo analyzer.

19-20. (canceled)

21. The biometric information measuring device according to claim 1, further comprising a waveform classifier for classifying according to types, basic waveforms contained in the measurement data displayed as apexcardiogram, and a display device for displaying or outputting the number of basic waveforms classified in each type.

22. The biometric information measuring device according to claim 1, wherein full waveform analysis of the basic waveform selected based on the basic waveform classification result by the waveform classifier, or input information from the outside of the data processor is performed.

23. (canceled)

24. A biometric information measuring system for measuring and recording the heartbeat of a living body, the biometric information measuring system comprising in the biometric information measuring device as defined in claim 1, a measurement system for determination of the health condition of a living body to be measured, wherein the measurement system is constituted by using the heartbeat sensor, and the data processor, and also using a health condition determination method for the living body using the first to the third characteristic-point determinator, at least one of the five determinators of the first to the fifth waveform determinators, and a health condition determinator for the living body to be measured.

25-27. (canceled)

28. A biometric information measurement system for measuring the heartbeat of a living body; the biometric information measuring system using a heartbeat sensor for measuring motion or pressure of a part of the living body at which the heartbeat is to be detected, based on change in the position of the part or change in the pressure;
wherein the heartbeat sensor is a light wave receiver element.

29. (canceled)
